SEQUENCE LISTING (32 sequences)

SEQ ID NO: 1 - protein, 30 aa, synthetic construct
  REGION 1..30: note = Description of sequence: peptide, name: alpha-CGRP8-37 amino acid sequence
VTHRLAGLLSRSGGVVKNNFVPTNVGSKAF

SEQ ID NO: 2 - DNA, 90 bp, synthetic construct
  misc_feature 1..90: note = Description of sequence: oligonucleotide, name: alpha-CGRP8-37 nucleic acid sequence
  source 1..90: mol_type = other DNA
gtgacccatcgcctggcgggcctgctgagccgcagcggcggcgtggtgaaaaacaactttgtgccgaccaacgtgggcagcaaagcgttt

SEQ ID NO: 3 - protein, 62 aa, synthetic construct
  REGION 1..62: note = Description of sequence: peptide, name: ssPAM amino acid sequence
MAGRVPSLLVLLVFPSSCLAFRSPLSVFKRFKETTRPFSNECLGTTRPVVPIDSSDFALDIR

SEQ ID NO: 4 - DNA, 186 bp, synthetic construct
  misc_feature 1..186: note = Description of sequence: oligonucleotide, name: ssPAM nucleic acid sequence
  source 1..186: mol_type = other DNA
atggcgggccgcgtgccgagcctgctggtgctgctggtgtttccgagcagctgcctggcgtttcgcagcccgctgagcgtgtttaaacgctttaaagaaaccacccgcccgtttagcaacgaatgcctgggcaccacccgcccggtggtgccgattgatagcagcgattttgcgctggatattcgc

SEQ ID NO: 5 - protein, 30 aa, synthetic construct
  REGION 1..30: note = Description of sequence: peptide, name: beta-CGRP8-37
VTHRLAGLLSRSGGMVKSNFVPTNVGSKAF

SEQ ID NO: 6 - protein, 4 aa, synthetic construct
  REGION 1..4: note = Description of sequence: peptide, name: linker
GGGG

SEQ ID NO: 7 - protein, 5 aa, synthetic construct
  REGION 1..5: note = Description of sequence: peptide, name: linker
GGGGG

SEQ ID NO: 8 - protein, 80 aa, synthetic construct
  REGION 1..80: note = Description of sequence: peptide, name: linker
  REGION 1..8: note = repeating linker subunit
  VARIANT 9..16: note = These positions may be absent as a unit: if one position with the segment is absent all positions within the unit are absent
  VARIANT 17..24: note = These positions may be absent as a unit: if one position with the segment is absent all positions within the unit are absent
  VARIANT 25..32: note = These positions may be absent as a unit: if one position with the segment is absent all positions within the unit are absent
  VARIANT 33..40: note = These positions may be absent as a unit: if one position with the segment is absent all positions within the unit are absent
  VARIANT 41..48: note = These positions may be absent as a unit: if one position with the segment is absent all positions within the unit are absent
  VARIANT 49..56: note = These positions may be absent as a unit: if one position with the segment is absent all positions within the unit are absent
  VARIANT 57..64: note = These positions may be absent as a unit: if one position with the segment is absent all positions within the unit are absent
  VARIANT 65..72: note = These positions may be absent as a unit: if one position with the segment is absent all positions within the unit are absent
  VARIANT 73..80: note = These positions may be absent as a unit: if one position with the segment is absent all positions within the unit are absent
GGGKGGGGGGGKGGGGGGGKGGGGGGGKGGGGGGGKGGGGGGGKGGGGGGGKGGGGGGGKGGGGGGGKGGGGGGGKGGGG

SEQ ID NO: 9 - protein, 80 aa, synthetic construct
  REGION 1..80: note = Description of sequence: peptide, name: linker
  REGION 1..8: note = repeating linker subunit
  VARIANT 9..16: note = These positions may be absent as a unit: if one position with the segment is absent all positions within the unit are absent
  VARIANT 17..24: note = These positions may be absent as a unit: if one position with the segment is absent all positions within the unit are absent
  VARIANT 25..32: note = These positions may be absent as a unit: if one position with the segment is absent all positions within the unit are absent
  VARIANT 33..40: note = These positions may be absent as a unit: if one position with the segment is absent all positions within the unit are absent
  VARIANT 41..48: note = These positions may be absent as a unit: if one position with the segment is absent all positions within the unit are absent
  VARIANT 49..56: note = These positions may be absent as a unit: if one position with the segment is absent all positions within the unit are absent
  VARIANT 57..64: note = These positions may be absent as a unit: if one position with the segment is absent all positions within the unit are absent
  VARIANT 65..72: note = These positions may be absent as a unit: if one position with the segment is absent all positions within the unit are absent
  VARIANT 73..80: note = These positions may be absent as a unit: if one position with the segment is absent all positions within the unit are absent
GGGNGSGGGGGNGSGGGGGNGSGGGGGNGSGGGGGNGSGGGGGNGSGGGGGNGSGGGGGNGSGGGGGNGSGGGGGNGSGG

SEQ ID NO: 10 - protein, 80 aa, synthetic construct
  REGION 1..80: note = Description of sequence: peptide, name: linker
  REGION 1..8: note = repeating linker subunit
  VARIANT 9..16: note = These positions may be absent as a unit: if one position with the segment is absent all positions within the unit are absent
  VARIANT 17..24: note = These positions may be absent as a unit: if one position with the segment is absent all positions within the unit are absent
  VARIANT 25..32: note = These positions may be absent as a unit: if one position with the segment is absent all positions within the unit are absent
  VARIANT 33..40: note = These positions may be absent as a unit: if one position with the segment is absent all positions within the unit are absent
  VARIANT 41..48: note = These positions may be absent as a unit: if one position with the segment is absent all positions within the unit are absent
  VARIANT 49..56: note = These positions may be absent as a unit: if one position with the segment is absent all positions within the unit are absent
  VARIANT 57..64: note = These positions may be absent as a unit: if one position with the segment is absent all positions within the unit are absent
  VARIANT 65..72: note = These positions may be absent as a unit: if one position with the segment is absent all positions within the unit are absent
  VARIANT 73..80: note = These positions may be absent as a unit: if one position with the segment is absent all positions within the unit are absent
GGGCGGGGGGGCGGGGGGGCGGGGGGGCGGGGGGGCGGGGGGGCGGGGGGGCGGGGGGGCGGGGGGGCGGGGGGGCGGGG

SEQ ID NO: 11 - protein, 50 aa, synthetic construct
  REGION 1..50: note = Description of sequence: peptide, name: linker
  REGION 1..5: note = repeating linker subunit
  VARIANT 6..10: note = These positions may be absent as a unit: if one position with the segment is absent all positions within the unit are absent
  VARIANT 11..15: note = These positions may be absent as a unit: if one position with the segment is absent all positions within the unit are absent
  VARIANT 16..20: note = These positions may be absent as a unit: if one position with the segment is absent all positions within the unit are absent
  VARIANT 21..25: note = These positions may be absent as a unit: if one position with the segment is absent all positions within the unit are absent
  VARIANT 26..30: note = These positions may be absent as a unit: if one position with the segment is absent all positions within the unit are absent
  VARIANT 31..35: note = These positions may be absent as a unit: if one position with the segment is absent all positions within the unit are absent
  VARIANT 36..40: note = These positions may be absent as a unit: if one position with the segment is absent all positions within the unit are absent
  VARIANT 41..45: note = These positions may be absent as a unit: if one position with the segment is absent all positions within the unit are absent
  VARIANT 46..50: note = These positions may be absent as a unit: if one position with the segment is absent all positions within the unit are absent
GPNGGGPNGGGPNGGGPNGGGPNGGGPNGGGPNGGGPNGGGPNGGGPNGG

SEQ ID NO: 12 - protein, 40 aa, synthetic construct
  REGION 1..40: note = Description of sequence: peptide, name: linker
  REGION 1..4: note = repeating linker subunit
  VARIANT 5..8: note = These positions may be absent as a unit: if one position with the segment is absent all positions within the unit are absent
  VARIANT 9..12: note = These positions may be absent as a unit: if one position with the segment is absent all positions within the unit are absent
  VARIANT 13..16: note = These positions may be absent as a unit: if one position with the segment is absent all positions within the unit are absent
  VARIANT 17..20: note = These positions may be absent as a unit: if one position with the segment is absent all positions within the unit are absent
  VARIANT 21..24: note = These positions may be absent as a unit: if one position with the segment is absent all positions within the unit are absent
  VARIANT 25..28: note = These positions may be absent as a unit: if one position with the segment is absent all positions within the unit are absent
  VARIANT 29..32: note = These positions may be absent as a unit: if one position with the segment is absent all positions within the unit are absent
  VARIANT 33..36: note = These positions may be absent as a unit: if one position with the segment is absent all positions within the unit are absent
  VARIANT 37..40: note = These positions may be absent as a unit: if one position with the segment is absent all positions within the unit are absent
GGGSGGGSGGGSGGGSGGGSGGGSGGGSGGGSGGGSGGGS

SEQ ID NO: 13 - protein, 50 aa, synthetic construct
  REGION 1..50: note = Description of sequence: peptide, name: linker
  REGION 1..5: note = repeating linker subunit
  VARIANT 6..10: note = These positions may be absent as a unit: if one position with the segment is absent all positions within the unit are absent
  VARIANT 11..15: note = These positions may be absent as a unit: if one position with the segment is absent all positions within the unit are absent
  VARIANT 16..20: note = These positions may be absent as a unit: if one position with the segment is absent all positions within the unit are absent
  VARIANT 21..25: note = These positions may be absent as a unit: if one position with the segment is absent all positions within the unit are absent
  VARIANT 26..30: note = These positions may be absent as a unit: if one position with the segment is absent all positions within the unit are absent
  VARIANT 31..35: note = These positions may be absent as a unit: if one position with the segment is absent all positions within the unit are absent
  VARIANT 36..40: note = These positions may be absent as a unit: if one position with the segment is absent all positions within the unit are absent
  VARIANT 41..45: note = These positions may be absent as a unit: if one position with the segment is absent all positions within the unit are absent
  VARIANT 46..50: note = These positions may be absent as a unit: if one position with the segment is absent all positions within the unit are absent
GGGGSGGGGSGGGGSGGGGSGGGGSGGGGSGGGGSGGGGSGGGGSGGGGS

SEQ ID NO: 14 - protein, 16 aa, synthetic construct
  REGION 1..16: note = Description of sequence: peptide, name: linker
GGGSGGGSGGGSGGGS

SEQ ID NO: 15 - protein, 16 aa, synthetic construct
  REGION 1..16: note = Description of sequence: peptide, name: linker
GGGGSGGGGGSGGGGS

SEQ ID NO: 16 - protein, 4 aa, synthetic construct
  REGION 1..4: note = Description of sequence: peptide, name: linker repeat unit
  REGION 1..4: note = This unit may repeat within a polypetide
GGGE

SEQ ID NO: 17 - protein, 5 aa, synthetic construct
  REGION 1..5: note = Description of sequence: peptide, name: linker repeat unit
  REGION 1..5: note = This unit may repeat within a polypetide
GGGGE

SEQ ID NO: 18 - protein, 4 aa, synthetic construct
  REGION 1..4: note = Description of sequence: peptide, name: linker repeat unit
  REGION 1..4: note = This unit may repeat within a polypetide
GGGD

SEQ ID NO: 19 - protein, 5 aa, synthetic construct
  REGION 1..5: note = Description of sequence: peptide, name: linker repeat unit
  REGION 1..5: note = This unit may repeat within a polypetide
GGGGD

SEQ ID NO: 20 - protein, 18 aa, synthetic construct
  REGION 1..18: note = Description of sequence: peptide, name: linker
GGEGGEGGEGGEGGEGGE

SEQ ID NO: 21 - protein, 4 aa, synthetic construct
  REGION 1..4: note = Description of sequence: peptide, name: linker repeat unit
  REGION 1..4: note = This unit may repeat within a polypetide
GGGK

SEQ ID NO: 22 - protein, 5 aa, synthetic construct
  REGION 1..5: note = Description of sequence: peptide, name: linker repeat unit
  REGION 1..5: note = This unit may repeat within a polypetide
GGGGK

SEQ ID NO: 23 - protein, 4 aa, synthetic construct
  REGION 1..4: note = Description of sequence: peptide, name: linker repeat unit
  REGION 1..4: note = This unit may repeat within a polypetide
GGGR

SEQ ID NO: 24 - protein, 5 aa, synthetic construct
  REGION 1..5: note = Description of sequence: peptide, name: linker repeat unit
  REGION 1..5: note = This unit may repeat within a polypetide
GGGGR

SEQ ID NO: 25 - protein, 15 aa, synthetic construct
  REGION 1..15: note = Description of sequence: peptide, name: linker
GGRGGRGGRGGRGGR

SEQ ID NO: 26 - protein, 5 aa, synthetic construct
  REGION 1..5: note = Description of sequence: peptide, name: linker repeat unit
  REGION 1..5: note = This unit may repeat within a polypetide
EAAAK

SEQ ID NO: 27 - DNA, 5505 bp, synthetic construct
  misc_feature 1..5505: note = Description of sequence: oligonucleotide, name: pAAV_ssPAM/CGRP8-37
  source 1..5505: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggcgggccgcgtgccgagcctgctggtgctgctggtgtttccgagcagctgcctggcgtttcgcagcccgctgagcgtgtttaaacgctttaaagaaaccacccgcccgtttagcaacgaatgcctgggcaccacccgcccggtggtgccgattgatagcagcgattttgcgctggatattcgccgcagcaaacgcagcgtgacccatcgcctggcgggcctgctgagccgcagcggcggcgtggtgaaaaacaactttgtgccgaccaacgtgggcagcaaagcgtttggccgccgccgccgcgatctgcaggcgggcagcggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 28 - DNA, 8574 bp, synthetic construct
  misc_feature 1..8574: note = Description of sequence: oligonucleotide, name: pLenti_ssPAM/CGRP8-37
  source 1..8574: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgaggctagccaactttgtatagaaaagttgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcaagtttgtacaaaaaagcaggctgccaccatggcgggccgcgtgccgagcctgctggtgctgctggtgtttccgagcagctgcctggcgtttcgcagcccgctgagcgtgtttaaacgctttaaagaaaccacccgcccgtttagcaacgaatgcctgggcaccacccgcccggtggtgccgattgatagcagcgattttgcgctggatattcgccgcagcaaacgcagcgtgacccatcgcctggcgggcctgctgagccgcagcggcggcgtggtgaaaaacaactttgtgccgaccaacgtgggcagcaaagcgtttggccgccgccgccgcgatctgcaggcgggcagctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 29 - protein, 37 aa, synthetic construct
  REGION 1..37: note = Description of sequence: peptide, name: CGRP
ACDTATCVTHRLAGLLSRSGGVVKNNFVPTNVGSKAF

SEQ ID NO: 30 - protein, 112 aa, synthetic construct
  REGION 1..112: note = Description of sequence: peptide, name: ssPAM-CGRP8-37 fragment
DAATMAGRVPSLLVLLVFPSSCLAFRSPLSVFKRFKETTRPFSNECLGTTRPVVPIDSSDFALDIRRSKRSVTHRLAGLLSRSGGVVKNNFVPTNVGSKAFGRRRRDLQAGS

SEQ ID NO: 31 - DNA, 338 bp, synthetic construct
  misc_feature 1..338: note = Description of sequence: oligonucleotide, name: ssPAM-CGRP8-37 fragment
  source 1..338: mol_type = other DNA
atcgatgccgccaccatggctggccgcgtccctagcctgctagttctccttgtttttccaagcagctgtttggctttccgaagcccactttctgtctttaagaggtttaaagaaactaccagaccattttccaatgaatgtcttggtaccaccagacccgtagttcctattgattcatcagattttgcattggatattcgcagatctaaacgatctgtgactcatcggctggcaggcttgtgagcagatcagggggtgtggtgaagaacaactttgtgcccaccaatgtgggttccaaagcctttggcaggcgccgcagggaccttcaagccggatcc

SEQ ID NO: 32 - protein, 29 aa, synthetic construct
  REGION 1..29: note = Description of sequence: peptide, name: CGRP 8-37
THRLAGLLSRSGGVVKNNFVPTNVGSKAF

(19) United States
(12) Patent Application Publication
(10) Pub. No.: US 2026/0263524 A1
(43) Pub. Date: Sep. 10, 2026

(54) CGRP ANTAGONIST COMPOSITIONS AND METHODS OF USE THEREOF

(71) Applicant: University of Miami, Miami, FL (US)

(72) Inventors: Jacqueline Sagen, Miami, FL (US); Stanislava Jergova, Miami, FL (US); Dimitrios Kouroupis, Miami, FL (US); Shyam Gajavelli, Miami, FL (US)

(21) Appl. No.: 19/442,367

(22) Filed: Jan. 7, 2026

Related U.S. Application Data
(60) Provisional application No. 63/768,212, filed on Mar. 7, 2025.

(51) Int. Cl. A61K 35/28; A61P 29/00; C07K 14/575; C12N 5/0775; C12N 15/861; C12N 15/864; C12N 15/867

(52) U.S. Cl. CPC A61K 35/28; A61P 29/00; C07K 14/57527; C12N 5/0667; C12N 15/861; C12N 15/8645; C12N 15/867; C07K 2319/02; C12N 2509/00; C12N 2710/10043; C12N 2740/15043; C12N 2750/14143

(57) ABSTRACT

The present disclosure provides AAV-delivered CGRP antagonist formulated in mesenchymal stem/stromal cell-derived extracellular vesicle compositions and kits for treating and/or preventing pain, including but not limited to chronic pain, and methods of use thereof.

### CROSS-REFERENCE TO RELATED APPLICATIONS

This application claims priority to, and the benefit of U.S. Provisional Patent Application No. 63/768,212, filed Mar. 7, 2025, which is incorporated by reference herein in its entirety.

### GOVERNMENT SUPPORT CLAUSE

This invention was made with Government support under Grant No. 5R21AR080388-02 awarded by the National Institutes of Health. The Government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jan. 6, 2026, is entitled “11348-070US1_ST26.xml”, and is 87,641 bytes in size.

### FIELD OF THE INVENTION

The present invention relates to improved calcitonin gene-related antagonist peptide (aCGRP) compositions and methods for treating pain, chronic pain, and inflammation.

### BACKGROUND OF THE INVENTION

Calcitonin gene-related peptide (CGRP) is produced by neurons in the dorsal root ganglia and trigeminal ganglia and thought to play a key role in nociceptive neurotransmission in the spinal dorsal horn and trigeminal dorsal horn. CGRP is understood to be a primary peptide mediator in both central and peripheral terminals of nociceptive primary afferents and has been shown to contribute to the pathology of peripheral inflammatory pain such as joint osteoarthritis, peripheral and central neuropathic pain resulting from injury to peripheral nerve and spinal cord, and migraine headache.

Long-term management of persistent and recurring long-term pain syndromes are particularly challenging to treat using repeated systemic drug administration due to the occurrence of off-target side effects, tolerance, and potential abuse. In addition, chronic pain including neuropathic pain is poorly managed by any current therapeutic approach and current pharmacologic approaches for recurrent migraine often result in rebound medication overuse headache (MOH). CGRP has long been recognized as a potential target for intervention in pain, particularly for headache.

Although synthetic CGRP antagonists (e.g. telcagepant) have been tested in the clinic, several have been discontinued due to systemic liver toxicity. Other versions are still in clinical development, but only for intermittent use. There are also currently CGRP receptor-targeted monoclonal antibodies in development. The efficacy of anti-CGRP receptor antibodies, however, may be limited by their lack or oral bioavailability.

There is a significant unmet need for new therapeutics to treat pain-related disorders.

### SUMMARY OF THE INVENTION

It has been surprisingly and unexpectedly discovered that a gene therapy comprising mesenchymal stem cell small extracellular vesicles (MSC sEVs) which are engineered to produce calcitonin gene-related peptide (CGRP) antagonist (including but not limited to CGRP8-37), can overcome current treatment limitations by providing direct targeting of the pain transmission system, obviating systemic side effects and bioavailability issues. In some embodiments, the gene therapy comprises CD10-enriched, substance P-degrading MSC sEVs engineered to produce a CGRP antagonist, for example including but not limited to CGRP8-37.

In addition, the approaches described herein surprisingly provide sustained local attenuation of abnormally activated CGRP nociceptive transmission, thus advantageously providing long-term pain relief after single administration and reducing the need for repeated pain medications.

The innovation of this disclosure is that selected CD10-enriched, substance P degrading MSC EVs engineered to produce CGRP antagonist (aCGRP-EVs), for example via an encapsulated viral vector, alleviate chronic pain and promote reversal of neurogenic inflammation.

Herein, human IFP-MSC cultures were transduced with an adeno-associated virus (AAV) viral vector carrying a GFP-labeled gene for a CGRP antagonist peptide (aCGRP) (see, e.g., FIG. 1A-1B of Example 1). The GFP positive aCGRP IFP-MSC were isolated by cell sorting and centrifugation (see, e.g., FIG. 1B of Example 1), and their sEVs' miRNA and protein cargos were assessed using multiplex methods.

Results surprisingly showed that purified aCGRP IFP-MSC cultures, which have a high percentage of CD10 expression (CD10High), yielded sEVs with cargo of 147 distinct MSC-related miRNAs (see, e.g., FIG. 3A of Example 1). Reactome analysis of miRNAs detected in these sEVs revealed strong involvement in the regulation of target genes involved in pathways that control pain, inflammation and cartilage homeostasis.

Further, protein array of the sEVs cargo demonstrated high presence of key immunomodulatory and reparative proteins (see, e.g., FIG. 5A of Example 1). Stimulated macrophages exposed to aCGRP IFP-MSC sEVs demonstrated a switch towards an alternate M2 status (see, e.g., FIG. 6A of Example 1). Also, stimulated cortical neurons exposed to aCGRP IFP-MSC sEVs modulate their molecular pain signaling profile (see, e.g., FIG. 6B of Example 1).

As described herein, sEVs of CD10-bound aCGRP IFP-MSC were engineered so that they could also target CGRP in addition to Substance P. MSC was transduced with a CGRP8-37 gene construct for the purpose of developing and evaluating innovative sEVs-based strategies for inflammation and pain reversal (see, e.g., FIG. 1A).

Collectively, these data suggest that yielded sEVs can putatively target CGRP in vivo, while containing potent anti-inflammatory and analgesic miRNA- and protein-based cargo, suggesting the promise for novel sEVs-based approaches to synergistically treat pain- or inflammation-related disorders, including but not limited to OA.

Separately, viral vectors encoding aCGRP and recombinant CGRP8-37 expressed therefrom (see, e.g., FIGS. 7B-7D 8A-8F, 9, and 10 of Example 2) were shown to exhibit excellent analgesic effects, relieving behavioral responses to pain by way of a variety of injection routes in multiple animal pain models (see, e.g., FIGS. 12A-12C, 13A-13B, 14A-14B, 15A-15D, and 16 of Example 2). In one aspect, provided herein is a viral vector comprising a nucleic acid sequence encoding for a fusion polypeptide as described herein. In some embodiments, the fusion protein comprises aCGRP and an ssPAM signal sequence. In some embodiments, the aCGRP comprises CGRP8-37. In some embodiments, the fusion polypeptide comprises a) a CGRP8-37 peptide comprising SEQ ID NO: 1 or a sequence at least 90% identical to SEQ ID NO: 1, and b) an ssPAM signal sequence comprising SEQ ID NO: 3 or a sequence at least 90% identical to SEQ ID NO: 3. In some embodiments, the fusion polypeptide comprises a) a CGRP8-37 peptide comprising SEQ ID NO: 32 or a sequence at least 90% identical to SEQ ID NO: 32, and b) an ssPAM signal sequence comprising SEQ ID NO: 3 or a sequence at least 90% identical to SEQ ID NO: 3. In some embodiments, the fusion protein comprises SEQ ID NO: 30, or an amino acid sequence at least about 90% identical thereto. In some embodiments the viral vector is a lentiviral vector. In some embodiments, the viral vector is an AAV vector. In some embodiments the AAV vector is AAV2.

This improvement provides a significant advance in the state of the art of pain and inflammation treatment.

Aspects of the disclosure provided herein include one or more of the following:
(i) a population of mesenchymal stem/stromal cell-derived small extracellular vesicles (MSC sEVs) comprising a viral vector encoding a calcitonin gene-related antagonist peptide (aCGRP);
(ii) a fusion polypeptide comprising a) a CGRP8-37 peptide comprising SEQ ID NO: 1 or a sequence at least 90% identical to SEQ ID NO: 1, and b) an ssPAM signal sequence comprising SEQ ID NO: 3 or a sequence at least 90% identical to SEQ ID NO: 3;
(iii) a viral vector, for example including but not limited to an AAV or a lentiviral vector, comprising a nucleic acid sequence encoding for a fusion polypeptide as described herein;
(iv) a pharmaceutical composition comprising a population of MSC sEVs as described herein, a fusion polypeptide as described herein, or a viral vector as described herein, and a pharmaceutically acceptable carrier;
(v) a method of treating pain in a subject in need thereof comprising administering a therapeutically effective amount of a pharmaceutical composition as described herein;
(vi) a method of treating headache in a subject in need thereof comprising administering a therapeutically effective amount of a pharmaceutical composition as described herein;
(vii) a method of treating osteoarthritis in a subject in need thereof comprising administering a therapeutically effective amount of a pharmaceutical composition as described herein;
(viii) a method for polarizing macrophages in a subject in need thereof, comprising administering a therapeutically effective amount of the pharmaceutical composition as described herein;
(ix) a method for reducing, preventing, or reversing inflammation in a subject in need thereof, comprising administering a therapeutically effective amount of the pharmaceutical composition as described herein;
(x) a method for reducing, preventing, or reversing neuroinflammation in a subject in need thereof, comprising administering a therapeutically effective amount of the pharmaceutical composition as described herein;
(xi) a method of obtaining a population of mesenchymal stem/stromal cell-derived small extracellular vesicles (MSC sEVs) comprising a viral vector encoding a calcitonin gene-related antagonist peptide (aCGRP), comprising: (i) obtaining a tissue sample of an infrapatellar fat pad (IFP) of a subject; (ii) mechanically dissecting the IFP tissue sample; (iii) washing the dissected IFP tissue sample in the presence of one or more digestion enzymes in a buffered solution; (iv) inactivating the one or more digestion enzymes; (v) culturing the digested IFP tissue sample in media to produce a population of IFP-MSCs; (vi) transducing the population of IFP-MSCs with one or more viral vectors encoding a CGRP8-37; and (vii) performing one or more purification steps to isolate the population of MSC sEVs, wherein the one or more purification steps are selected from filtering, centrifuging, or a combination thereof; and
(xii) a method of treating pain in a subject in need thereof, comprising administering to the subject a population of MSC sEV isolated as described herein.

A summary of embodiments of the invention is described in further detail below.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A shows gene construct of adenovirus vector containing the gene for CGRP8-37. The CGRP8-37 fragment was inserted downstream of the signal sequence of peptidylglycine-amidating monooxygenase (ssPAM). This arrangement facilitates the amidation and secretion of CGRP8-37.

FIG. 1B shows MSC were isolated from human infrapatellar fat pad (IFP), transduced with gene construct, and sorted by FACS cell sorting to generate aCGRP IFP-MSC. aCGRP IFP-MSC sEVs were isolated and characterized for their miRNA cargo. Functional assessment of aCGRP

IFP-MSC sEVs was performed by macrophage polarization and cortical neurons neuroinflammation assays.

FIG. 2A shows FACS sorting of GFP-labelled AAV CGRP8-37 transduced IFP-MSC (n=5). Cell sorting of transduced cells resulted in the purification of an aCGRP IFP-MSC subpopulation.

FIG. 2B shows aCGRP IFP-MSC showed similar fibroblast-like morphology, but lower clonogenic capacity (72+42 CFU-Fs) to non-transduced IFP-MSC.

FIG. 2C shows aCGRP IFP-MSC showed high expression levels of common MSC-defining markers (CD73, CD90, CD105, CD146, CD10, HLA-DR). CD10 expression was similarly high (>95%) whereas CD146 showed reduced expression compared to non-transduced IFP-MSC.

FIG. 2D shows aCGRP IFP-MSC sEVs showed high purity and <200 nm sizes. CD63+-selected sEVs showed high positivity for CD9 marker.

FIG. 2E shows immunostaining of HEK 293 cells after incubation with aCGRP IFP-MSC SEVs. Top: Detection of AAV protein in HEK cells incubated with different concentration range of SEVs, Middle: Detection of CGRP protein in HEK cells incubated with different concentration range of sEVs, and bottom: Lower magnification of HEK cells after incubation with sEVs from recombinant MSC (left) and control, sEVs from no recombinant MSC. Plots: Top: Integrated density of AAV signal. aCGRP IFP-MSC sEVs were used at 30 μg/mL (30), 50 μg/mL (50) and 80 μg/mL (80) concentrations. *p<0.05, **p<0.01, and bottom: Integrated density of CGRP signal. *p<0.05, **p<0.01, ***p<0.001.

FIG. 3A shows 147 distinct miRNA were present in aCGRP IFP-MSC sEVs (n=2). Nineteen highly present miRNAs are included in black-bordered box.

FIG. 3B shows miRNAs present in aCGRP IFP-MSC sEVs were involved in the regulation of numerous genes and pathways. Predominantly, miRNAs present in sEVs are involved in the regulation of TGF-β/Wnt/FGFR pathways. Putative miRNA interactomes were generated using a miRNet centric network visual analytics platform. The miRNA target gene data were collected from well-annotated database miRTarBase v8.0 and miRNA-gene interactome network refining was performed with 2.0 betweenness cut-off. Values (with 34 cycles cut-off point) were represented in a topology miRNA-gene interactome network using force atlas layout and hypergeometric test algorithm.

FIG. 4A shows that in aCGRP IFP-MSC sEVs (n=2), 19 miRNAs cargos were highly present. These distinct miRNAs regulate genes involved in the production of cytokines, the recruitment of monocytes, and cartilage homeostasis. From these miRNAs, 7 miRNAs were associated in previous studies with significant anti-inflammatory/immunomodulatory effects in vitro and in vivo. Putative miRNA interactomes were generated using a miRNet centric network visual analytics platform. The miRNA target gene data were collected from well-annotated database miRTarBase v8.0 and miRNA-gene interactome network refining was performed with 2.0 betweenness cut-off. Values (with 34 cycles cut-off point) were represented in a topology miRNA-gene interactome network using force atlas layout and hypergeometric test algorithm.

FIG. 4B shows that in silico analysis reveals a functional correlation of identified miRNAs in aCGRP IFP-MSC sEVs with genes involved in M2 macrophage polarization, immunomodulatory and pain signaling, and cartilage homeostasis. The miRDB online database for prediction of functional miRNA targets has been used to correlate highly expressed target genes in macrophages with specific miRNAs identified by aCGRP IFP-MSC sEV miRNA profiling. MirTarget prediction scores are in the range of 0-100% probability, and candidate transcripts with scores ≥50% are presented as predicted miRNA targets in miRDB.

FIG. 5A shows multiple immunomodulatory and reparative molecules secreted as a cargo of aCGRP IFP-MSC sEVs (n=2). sEVs showed presence of key immunomodulatory molecules including TIMP-2, IL-8, MCP-1, IL-6, ICAM-1, sTNF-RI, MIP-1β, IL-10, and IP-10. In parallel, key reparative molecules including HGF, VEGF, EGFR, IGFBP-1, BFGF, and IGFBP-6 showed presence in aCGRP IFP-MSC sEVs. These proteins are listed in descending order based on their presence levels within sEVs. The miRDB online database for prediction of functional miRNA targets has been used to correlate highly expressed target genes in macrophages with specific miRNAs identified by aCGRP IFP-MSC sEV miRNA profiling.

FIG. 5B shows that in terms of biological processes, various categories were highly affected and presented as % of proteins involved in a category to the total proteins detected. aCGRP IFP-MSC sEVs protein cargo have effects on PI3K-Akt signaling pathway (59%), MAPK signaling pathway (55%), Ras signaling pathway (52%), Rap1 signaling pathway (43%), cytokine-cytokine receptor interaction (24%), and Jak-STAT signaling pathway (19%). MirTarget prediction scores are in the range of 0-100% probability, and candidate transcripts with scores ≥50% are presented as predicted miRNA targets in miRDB.

FIG. 6A shows PMA/IO-stimulated THP-1 showed similar morphology with and without aCGRP IFP-MSC sEVs treatment (n=2). Upon exposure to aCGRP IFP-MSC SEVs, PMA/IO-stimulated THP-1 gene expression analysis showed a strong shift towards M0/M2 macrophage polarization. Notably, exposure to aCGRP IFP-MSC sEVs induced significant expression of key M2-polarization markers such as CD200R1, BMP7, IRF4, IL10, and IL12A.

FIG. 6B shows TIC-stimulated cortical neurons showed similar morphology with and without aCGRP IFP-MSC sEVs treatment (n=2). Upon exposure to aCGRP IFP-MSC sEVs, their molecular profiling indicated an overall reduced neuroinflammatory profile upon exposure to aCGRP IFP-MSC sEVs. From 84 genes, only 14 (SLC6A2, IL1B, PTGS1, EDN1, SCN11A, CCR2, TLR2, GDNF, CD4, OPRD1, ACE, ALOX5, PTGES, CCL12) showed increased expression (>2-fold) compared to TIC-stimulated cortical neurons alone. Bars indicate the >2-fold expressed genes. Interestingly, 4 major genes (MAPK8, CD200, MAPK1, PTGES3) involved in neuropathic pain were highly down-regulated (>2-fold) upon exposure to aCGRP IFP-MSC sEVs.

FIG. 7A shows the sequence of the ssPAM-CGRP8-37 fragment. The restriction enzyme sites of ClaI, BgIII, and BamHI are shown. In some embodiments, the ssPAM-CGRP8-37 fragment amino acid sequence comprises SEQ ID NO: 30 as disclosed herein. In some embodiments, the ssPAM-CGRP8-37 fragment nucleic acid sequence comprises SEQ ID NO: 31 as disclosed herein. In some embodiments, the CGRP (8-37) sequence comprises SEQ ID NO: 1 as disclosed herein. In some embodiments, the CGRP sequence comprises SEQ ID NO: 29 as disclosed herein.

FIG. 7B shows cDNA gel electrophoresis of ClaI-BamHI digested AAV-sspam-CGRP8-37-EGFP construct shows fragments of AAV vector (5.9 Kb) and sspam-CGRP8-37 insert (0.34 kb), confirming correct location of the insert and stable design of the plasmid.

FIG. 7C shows gel electrophoresis of RT-PCR isolated from cells cultures and amplified with template for CGRP 8-37 to detect its presence in the supernatant. HEK293T cells were transfected with AAV vector (lane 1) and AAV-sspam-CGRP8-37-EGFP (lane 2; SEQ ID NO: 27). Recombinant insert, sspam-CGRP8-37 (340 bp) was only detected in the cells transfected by recombinant AAV vector.

FIG. 7D shows gel electrophoresis of RT-PCR isolated from cells cultures and amplified with template for CGRP 8-37 to detect its presence in the supernatant. Rat embryonic neuronal progenitor cells were transfected with Lenti-sspam-CGRP8-37 (SEQ ID NO: 28). The CGRP8-37 insert detected only in the supernatant of transfected cells with CGRP8-37 template used for PCR amplification (lane 2).

FIGS. 8A-8F show confocal images of transfected HEK 293T cells expressing the CGRP8-37-EGFP construct stained for various targets.

FIG. 8A shows nuclear stain DAPI.

FIG. 8B shows anti-GFP stain.

FIG. 8C shows anti-CGRP stain.

FIG. 8D shows colocalization of GFP with CGRP in the perinuclear region, which suggests successful production of recombinant CGRP8-37-EGFP peptide.

FIG. 8E shows anti-Giantin stain.

FIG. 8F shows the overlay of GFP and CGRP signals with Giantin, a Golgi marker, suggesting synthesis of CGRP8-37 in secretory pathway.

FIG. 9 shows an orthogonal view of a confocal image of transfected HEK 293T cells expressing the CGRP8-37-KR-EGFP construct stained anti-CGRP antibody and nuclear stain DAPI shows synthesis and separation of CGRP 8-37-EGFP and CGRP (filled arrows). A region without colocalization in the peripheral cytoplasm is predominantly stained (open arrow) and less alternative fluorescence stain (arrows on x and y axes). The CGRP8-37 and EGFP have an intervening dipeptide that facilitates the separation of the single fusion protein into its components.

FIG. 10 shows FLISA detection of recombinant CGRP8-37 peptide in the supernatant of 293T cells transfected with recombinant and nonrecombinant AAV vector with CGRP8-37 fragment.

FIGS. 11A-11B show behavioral evaluation of analgesic effect of supernatant from Lenti-ssPAM-CGRP8-37 transfected cells in the formalin model in rats. Acute inflammation was induced by 5% formalin injected into rat hind paw. The pain related behavior (flinching, licking of the paw) was observed and recorded for 60 min post injection. Supernatant/control CGRP8-37 peptide/saline was injected intrathecally 15 min before the formalin injection.

FIG. 11A shows formalin induced behavior was fully developed in saline injected animals (line) and significantly reduced in animals injected with control peptide (line) and cell supernatant (line), indicating the presence of active peptide in the supernatant.

FIG. 11B shows further analysis of Phase I (0-5 min) and Phase 2 (20-60 min) shows the significant reduction of pain related behavior in animals injected with control peptide and supernatant. Data expressed as mean±SEM; *p<0.05, **p<0.01; significantly different from control (saline).

FIGS. 12A-12C show intrathecal injections of supernatant from CGRP8-37-transduced HEK cells reduce neuropathic pain induced by chronic constriction injury (CCI) of the sciatic nerve. These findings suggest successful production of the active CGRP8-37 from the engineered CGRP8-37 cDNA as described herein.

FIG. 12A shows a reduction of cold allodynia in CCI rats treated with CGRP8-37 supernatant that was comparable to intrathecal injection of authentic CGRP peptide, whereas no alleviation of pain behavior was seen in untreated CCI rats. (*p<0.05, ** **p<0.01, *p<0.001 vs CCI).

FIG. 12B shows a reduction of tactile allodynia in CCI rats treated with CGRP8-37 supernatant that was comparable to intrathecal injection of authentic CGRP peptide, whereas no alleviation of pain behavior was seen in untreated CCI rats. (*p<0.05, ** **p<0.01, *p<0.001 vs CCI).

FIG. 12C shows a reduction of mechanical hyperalgesia in CCI rats treated with CGRP8-37 supernatant that was comparable to intrathecal injection of authentic CGRP peptide, whereas no alleviation of pain behavior was seen in untreated CCI rats. (*p<0.05, ** **p<0.01, *p<0.001 vs CCI).

FIGS. 13A-13B show lenti-sspam-CGRP8-37-EGFP viral vector intraspinal injection reduces spinal cord injury (SCI) induced pain. SCI rats were intraspinally injected with the either L-sspam-CGRP8-37EGFP viral vector or control Lenti-EGFP into L3-L4 lumbar dorsal horn; sham animals were not injected.

FIG. 13A shows attenuation of mechanical hyperalgesia was observed by 6-7 wk post injection with gradual improvement of behavioral outcomes towards pre-injury levels by 9 weeks post-SCI. *<0.05, **<0.01; lentiCGRP8-37EGFP viral vector compared with control Lenti-EGFP (injury).

FIG. 13B shows attenuation of cold allodynia was observed by 6-7 wk post injection with gradual improvement of behavioral outcomes towards pre-injury levels by 9 weeks post-SCI. *<0.05, **<0.01; lentiCGRP8-37EGFP viral vector compared with control Lenti-EGFP (injury).

FIGS. 14A-14B show AAV-sspam-CGRP8-37-EGFP intraspinal injection reduces CCI pain. Chronic constriction injury was induced in animals to mimic pain after peripheral neuropathy. AAV construct with either CGRP8-37 or control GFP gene was injected intraspinally into lumbar L3-L4 spinal cord at 1 week post injury. The responses of animals to tactile (FIG. 14A) and cold (FIG. 14B) stimuli were evaluated.

FIG. 14A shows attenuation of tactile allodynia was observed in animals treated with CGRP8-37 construct. *p<0.05, **p<0.01 vs GFP.

FIG. 14B shows attenuation of cold hyperalgesia was observed in animals treated with CGRP8-37 construct. *p<0.05, **p<0.01 vs GFP.

FIGS. 15A-15D show AAV-sspam-CGRP8-37-EGFP intraspinal injection reduces SCI pain. Spinal cord clip injury was induced in animals to mimic below level SCI pain. AAV construct with either CGRP8-37 or control GFP gene was injected intraspinally into lumbar L3-L4 spinal cord at 4 weeks post injury. The responses of animals to tactile (FIG. 15A), cold (FIG. 15B), heat (FIG. 15C) stimuli, and locomotor scores (FIG. 15D) were evaluated.

FIG. 15A shows attenuation of tactile allodynia was observed in animals treated with CGRP8-37 construct. *p<0.05, **p<0.01 vs GFP.

FIG. 15B shows attenuation of heat hyperalgesia was observed in animals treated with CGRP8-37 construct. *p<0.05, **p<0.01 vs GFP.

FIG. 15C shows attenuation of cold hyperalgesia was observed in animals treated with CGRP8-37 construct. *p<0.05, **p<0.01 vs GFP.

FIG. 15D shows animals in both treatment groups show similar locomotor recovery (BBB score), confirming safety of the treatment.

FIG. 16 shows the analgesic effect of AAV-ssPAM-CGRP8-37 viral vector trigeminal injection in migraine model in rats. Animals were cannulated and inflammatory soup (IS) injected over the brain dura to induce migraine-like behavior. AAV-CGRP8-37 viral vector construct was injected into trigeminal ganglia 25 days post injection of IS. Conditioned place preference was evaluated in animals; time spent in the light side of the testing cage over the darker side was recorded before and after the treatment. Animals with IS injection only developed a strong preference towards the darker side (negative values, *p<0.05). Animals with IS injection treated with AAV-CGRP8-37 viral vector construct did not develop such behavior and their values were similar to control animals. Values in both treated and control animals were significantly different from untreated animals with IS injection. (#p<0.05, ##p<0.01). N=4-5.

FIG. 17 shows MSC isolated from the human infrapatellar fat pad (IFP), transduced with gene construct, and sorted by FACS cell sorting to generate aCGRP IFP-MSC. aCGRP IFP-MSC EVs were characterized for their miRNA and protein cargos. EVs were then functionally assessed by dorsal root ganglia (DRGs) neuroinflammation assay, chondropellets/synoviocytes co-culture assay, and acute synovial/IFP inflammation animal model.

FIG. 18 shows that acute synovial/IFP inflammation often seen in OA was generated by intra-articular injection of MIA into the right knee. Four days later, a single intra-articular injection of aCGRP EVs was performed. Behavioral analysis was performed at baseline, 3 days after MIA injection, and 3, 6, and 13 days post-EVs treatment.

FIG. 19A shows twenty-four distinct LncRNAs were present as cargo in aCGRP IFP-MSC EVs. Mean values were normalized to small nucleolar RNA SNORA73.

FIG. 19B shows lncRNAs present in aCGRP IFP-MSC EVs were involved in the regulation of important cell processes such as regulation of stem cell, cell cycle, cell proliferation, inflammation, bone regeneration, osteogenesis, and cell death. Putative interactomes were generated using a miRNet centric network visual analytics platform.

FIG. 20A shows dorsal root ganglia (DRGs) neuroinflammation assay. DRGs showed identical morphology with and without the addition of aCGRP IFP-MSC EVs treatment.

FIG. 20B shows TIC-stimulated DRGs molecular profiling indicated an overall reduced neuroinflammatory profile upon exposure to aCGRP IFP-MSC EVs. Dotted line marks the gene expression levels without EVs-treatment (TIC-stimulated DRGs alone). Out of the 84 genes, only 3 (Cala, Trpa1, and Scn11, highlighted) show increased expression (>2 fold) compared to TIC-stimulated DRGs alone. Five important genes (Mapk1, Mapk3, Mapk8, Tlr4, and Tnf highlighted dark grey) involved in neuroinflammation were highly downregulated (>2-fold) upon exposure to aCGRP IFP-MSC EVs. Scale bar: 100 μm. Magnification ×4.

FIG. 21 shows chondropellets/synoviocytes co-cultures under inflammatory conditions. Treatment of chondropellets/synoviocytes co-cultures with aCGRP IFP-MSC EVs led to upregulated expression of several genes in chondropellets with diverse roles in cartilage biology, some of which are closely tied to anabolic processes, while others are typically associated with catabolic or inflammatory functions. Dotted line marks the gene expression levels without EVs-treatment (TIC-stimulated chondropellets/synoviocytes alone). Bars: gene expression below the threshold, bars: gene expression above threshold.

FIG. 22A shows the tactile allodynia (Von-Frey filament tactile test) that evaluated the paw withdrawal threshold. aCGRP IFP-MSC EVs-treated knees showed lower allodynia compared to non-treated. Lines: aCGRP IFP-MSC EVs-treated animals, lines: diseased animals (negative control), lines: CD10High IFP-MSC EVs-treated animals (positive control). Individual points represent mean±SEM, *p<0.05.

FIG. 22B shows the cold allodynia (acetone test) which showed the % response of EVs-treated and non-treated knees to acetone exposure. aCGRP IFP-MSC EVs-treated knees showed lower % response compared to non-treated. Lines: aCGRP IFP-MSC EVs-treated animals, lines: diseased animals (negative control), lines: CD10High IFP-MSC EVs-treated animals (positive control). Individual points represent mean±SEM, *p<0.05.

FIG. 22C shows the knee bend test that evaluated the pain score upon knee bend. aCGRP IFP-MSC EVs-treated knees showed lower pain score compared to non-treated. Lines: aCGRP IFP-MSC EVs-treated animals, lines: diseased animals (negative control), lines: CD10High IFP-MSC EVs-treated animals (positive control). Individual points represent mean±SEM, *p<0.05.

FIG. 23A shows histochemical and immunohistochemical evaluation of chondroprotective and analgesic effects of aCGRP IFP-MSC EVs intra-articular administration in rat knee joint. Masson's trichrome staining, revealed pronounced collagen disruption (staining) in disease cartilage (top panel) compared to healthy (middle panel) and treatment (bottom panel) groups. Immunohistochemical staining for PRG4 illustrated upregulation of PRG4 expression to the intermediate zone in MIA only group compared to treatment and healthy group. Immunohistochemical staining for SP illustrated greater signal in diseased animals compared to treatment and healthy groups. Scale bar: 30 μm. Magnification ×10.

FIG. 23B shows treatments with aCGRP IFP-MSC EVs significantly improved collagen preservation, with a lower ratio of discolored to normal collagen. Treatment with aCGRP IFP-MSC EVs significantly lowered quantity of PRG4 expressed at the second sacrifice. Immunofluorescence analysis showed elevated SP levels in diseased cartilage compared to healthy controls. Although EVs-treated samples showed a non-significant reduction in SP, these trends suggest potential modulation of neurogenic inflammation. Bars: healthy knees, bars: disease knees sacrifice 1 (Sac1) and sacrifice 2 (Sac2), bars: EVs-treated knees sacrifice 1 (Sac1) and sacrifice 2 (Sac2).

FIG. 24 shows neuroinflammation qPCR array of 84 genes involved in the transduction, maintenance, and modulation of pain responses. Dorsal root ganglia were extracted on day 26 from non-treated and intraarticularly treated (FP CD10Low EVs, FP CD10High EVs, FP aCGRP/CD10High EVs) rats. FP CD10Low infusion resulted in reduced neuroinflammatory response with only 31 genes upregulated compared to non-treated group (upper graph). FP CD10High infusion resulted in reduced neuroinflammatory response with only 23 genes upregulated compared to non-treated group (middle graph). FP aCGRP/CD10High EVs resulted in reduced neuroinflammatory response with only 16 genes upregulated compared to non-treated group (bottom graph). Shaded bars indicate upregulated genes compared to non-treated group.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

The analgesic effect of CGRP8-37, a competitive antagonist of CGRP, has been shown in several pain models and is further described in the aspects and Working Examples herein.

### Definitions

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, inhibitors, isomers, fragments, analogs, and the like. When the term “composition” is used, then, or when a particular composition is specifically identified, it is to be understood that the term includes the composition per se as well as pharmaceutically acceptable, pharmacologically active vector (such as, for example, a viral vector or an AAV vector), polynucleotide, salts, esters, amides, proagents, conjugates, active metabolites, isomers, fragments, analogs, etc.

A “therapeutic composition” refers to at least one substance, molecule, or compound suitable for administering to a subject, wherein the composition further includes a pharmaceutical carrier. A non-limiting example include a therapeutic composition comprises an inhibitor or an antibodies.

As used herein, the term “agent” or “therapeutic agent” refers to a living organism or biological substance, such as a virus, chemical, toxin, or antibody, that can be designed to purposefully fulfill a biological function or action.

As used herein, a “therapeutic regimen” refers to a structured treatment plan or strategy designed to improve and maintain health. Generally, a therapeutic regimen will be designed, prescribed, and/or administered by a licensed medical practitioner. The therapeutic regimen generally specifies the treatment dosage, the treatment scheduling, and the duration of the treatment. In some embodiments, the therapeutic regimen comprises one or more therapeutic compositions. In some embodiments, the therapeutic regimen comprises one or more therapeutic agents. In some embodiments, the therapeutic regimen comprises any combination of therapeutic compositions and therapeutic agents, such as for example the combination of an inhibitor and an antibody. In some embodiments, a therapeutic regimen comprises modifying, continuing, and/or initiating at least one therapeutic agent and/or therapeutic composition. In some embodiments, a therapeutic regimen comprises treating and/or preventing a disease, disorder, and/or condition.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

By “reduce” or other forms of the word, such as “reducing” or “reduction,” means lowering of an event or characteristic (e.g., viral load). It is understood that this is typically in relation to some standard or expected value, in other words it is relative, but that it is not always necessary for the standard or relative value to be referred to. For example, “reduces viral load” means reducing the amount of virus or virus particles in an infected person's blood relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. Thus, the subject can be a human or veterinary patient. The term “patient” refers to a subject under the treatment of a clinician, e.g., physician.

The term “treatment” refers to the medical management of a patient with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intra-DRG, intrahepatic, intralesional, and intracranial injections or infusion techniques. In some embodiments, the injection is in the trigeminal (gasserian) ganglion.

A “vector” as used herein refers to any particle or composition used as a vehicle to artificially carry a foreign nucleic acid sequence into another cell, where it can be replicated and/or expressed. Examples of vectors include plasmids, viral vectors, cosmids, and artificial chromosomes.

As used herein a “viral vector” refers to a tool used in molecular biology to deliver genetic material (including DNA, RNA, and any other nucleic acid variations thereof) into a cell. This process is performed either inside a living organism or in cell culture. The viral genome is engineered to incorporate a desired gene or gene product, and following transduction, or transfer, of the virus into the host, said gene or gene product is expressed within the host.

An “adeno-associated virus” or an “AAV” as used herein refers to a small virus belonging to the genus Dependoparvovirus which are replicative defective, non-enveloped viruses with linear single-stranded DNA. These viruses are commonly used for creating viral vectors for gene therapy, wherein said viruses can infect dividing and quiescent cells and persist in an extrachromosomal state without integrating into the host genome. AAVs can be engineered to express desired genes or gene products such as mRNA, shRNA, or miRNAs to overexpress or silence a target gene.

A “variant” or a “derivative” of a particular inhibitor may be defined as a chemical or molecular compound having at least 50% identity to a parent or original inhibitor. In some embodiments a variant inhibitor may show, for example, at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% or greater identity relative to a reference parent or original inhibitor.

A “virus” is a microscopic infectious agent that replicates only inside the living cells of an organism. Viruses can infect all life forms, including mammalian and non-mammalian animals, plants, and other microorganisms. A complete virus, also known as a virion, consists of nucleic acid genetic material surrounded by a protective coat of protein called a capsid. Virus can have a lipid envelope derived from the infected host cell membrane. In general, there are five morphological virus types including helical, icosahedral, prolate, enveloped, and complex virus. A virus can either have a DNA or RNA genome, though a vast majority have RNA genomes. Irrespective of the type of nucleic acid genome, a viral genome can be either a single-stranded genome or a double-stranded genome.

As used herein, the term “calcitonin gene-related peptide” and “CGRP” refers to any form of calcitonin gene-related peptide and variants thereof that retain at least part of the activity of CGRP. For example, CGRP may be α-CGRP or β-CGRP. As used herein, CGRP includes all mammalian species of native sequence CGRP, e.g., human, canine, feline, equine, and bovine.

### Compositions

In one aspect, provided herein is a population of mesenchymal stem/stromal cell (MSC)-derived small extracellular vesicles (sEVs) comprising a viral vector encoding a calcitonin gene-related antagonist peptide (aCGRP).

Mesenchymal stem/stromal cells can be derived from a number of cell sources. In some embodiments, the MSC sEVs are derived from infrapatellar fat pad (IFP). Ins some embodiments, the MSCs are isolated from placental derivatives or from bone marrow, or obtained from a number of different sources, including bone marrow mononuclear cells, umbilical cord blood, adipose tissue, endometrium, placental tissue, synovium, plugs of femoral head cancellous bone pieces, obtained from patients with degenerative joint disease during hip or knee replacement surgery, or from aspirated marrow obtained from normal donors and oncology patients who have marrow harvested for future bone marrow transplantation. In some embodiments, MSCs can be isolated from commercially available bone marrow aspirates, adipose tissue samples, and Wharton's jelly samples. Enrichment of MSCs within a population of cells can be achieved using methods known in the art including but not limited to FACS. See, for example, Han et al. (2019) Cells 8:886 and U.S. Pat. Nos. 9,901,600 and 9,085,755. The critical step involved in the isolation processes is the use of a specially prepared medium containing agents allowing for mesenchymal stem cell growth without differentiation, but also for the direct adherence of only the mesenchymal stem cells to the plastic or glass surface area of the culture dish.

Mesenchymal stem cells from human and other animal sources are also commercially available from, for example, Thermo Fisher Scientific, Inc., Accegen Biotechnology, and PromoCell GmbH.

The isolation and cultivation of the population of mesenchymal stem cells can be carried out under standard conditions for the cultivation of mammalian cells. Typically, the methods provided herein of expanding a population of mesenchymal stem cells are typically carried out at conditions (temperature, atmosphere) that are normally used for cultivation of cells of the species of which the cells are derived. For example, human mesenchymal stem cells are usually cultivated at 37° C. in air atmosphere with 5% CO2. In this context, it is noted that the in the provided methods the mesenchymal stem cells may be derived of any mammalian species, such as mouse, rat, guinea pig, rabbit, goat, horse, dog, cat, sheep, monkey or human, with mesenchymal stem cells of human origin being preferred in one embodiment.

Extracellular vesicles (EVs) are generated via the endosomal pathway, and secreted by numerous cells in response to their surrounding milieu. sEVs are nanometer-sized membranous particles that are immunologically inert and contain many constituents of a cell, including DNA, RNA, lipids, metabolites, and cytosolic and cell-surface proteins. MSC-derived sEVs mediate various biological functions attributed to MSC, such as tissue regeneration, intercellular communication, modulation of immunity and cell signaling. On this basis, sEVs can be manipulated and engineered as described herein to deliver robust therapeutic molecules targeting inflammation and pain reversal in vivo.

The isolation of sEVs has been described in the art (see, e.g., Kouroupis, D. et al. Human infrapatellar fat pad mesenchymal stem cells show immunomodulatory exosomal signatures. Sci Rep. 2022 Mar. 4; 12 (1): 3609, incorporated herein by reference). For example, IFP-MSC derived sEVs may be isolated from IFP-MSC conditioned medium by a step-wise ultracentrifugation method and CD63-immunomagnetic purification. Briefly, conditioned media from IFP-MSC groups cultured in exosome-depleted Ch-R medium can be filtered, for example through a 0.22 μm filter, to remove debris and large vesicles, and differentially centrifuged, for example for 2000×g for 10 min, 10,000×g for 30 min, and ultracentrifuged for 120,000×g for 16 hr. Pre-enriched exosome preparations can be incubated with a Dynabeads®-based Exosome-Human CD63 Isolation/Detection Reagent (Invitrogen) and, using a magnetic separator, exosome preparations can be further purified. Quantity and size determination can be performed by, for example, nanoparticle tracking analysis (NTA) (NanoSight NS300, Malvern). Exosome presence can be validated by the presence of several markers, including CD9 (Invitrogen) expression, in CD63+-gated particles by flow cytometry.

In some embodiments, greater than about 90%, greater than about 95%, greater than about 96%, greater than about 97%, greater than about 98%, or greater than about 99% of the MSC sEVs are bound to one or more MSC-defining markers, wherein the markers are selected from CD9, CD73, CD90, CD105, CD10, or a combination thereof.

Based on its enzymatic activity, CD10 anti-inflammatory effects have been recognized in other systems, while CD10 KO mice have an increased propensity to inflammatory conditions such as colitis. Significant CD10 enrichment was recently reported in IFP-MSCs stimulated (i.e., primed) with inflammatory/pro-fibrotic cocktails (TNFα/IFNγ/CTGF) or cultured regulatory-compliant media, and effective Substance P (SP) degradation capacity both in vitro and in vivo. A comparable SP degradative effect has been consistently found by the cells and their supernatant, abrogated by a CD10 inhibitor, suggesting an extracellular vesicle-mediated mechanism involving the release of CD10 from the cells.

In some embodiments, greater than about 90%, greater than about 95%, greater than about 96%, greater than about 97%, greater than about 98%, or greater than about 99% of the MSC sEVs are bound to one or more neprilysin (CD10) molecules. In some embodiments, greater than about 99% of the MSC sEVs are bound to one or more CD10 molecules.

In some embodiments, less than about 35%, less than about 30%, or less than about 25% of the MSC sEVs are bound to one or more CD146 molecules.

In some embodiments, the MSC sEVs have low expression of HLA-DR. In some embodiments, less than about 5%, less than about 4%, less than about 3%, less than about 2%, less than about 1%, or less than about 0.5% of the MSC sEVs are bound to one or more HLA-DR molecules. In some embodiments, the MSC sEVs have absent expression of HLA-DR.

In some embodiments, the MSC sEVs are capable of degrading Substance P (SP).

In addition to CGRP, Substance P (SP) participates in the modulation of local neurogenic inflammatory/immune responses. SP increases vascular permeability favoring immune cell infiltration, while directly affecting macrophage phenotypic polarization and migration to sites of inflammation. Despite the well-described roles of SP in nociception and neurogenic inflammation, little is known about how to effectively inhibit SP signaling activity. SP activity is regulated by the cell membrane-bound (ectoenzyme) neutral endopeptidase CD10/neprilysin, expressed in Mesenchymal Stem Cells (MSC) and specifically in infrapatellar fat pad-derived MSCs (IFP-MSCs) as described herein.

In addition to the established roles of Substance P (SP) in nociception, recent evidence supports its participation in the modulation of local neurogenic inflammatory/immune responses. SP increases vascular permeability favoring immune cell infiltration, while directly affecting macrophage phenotypic polarization and migration to sites of inflammation. Meanwhile, regulation of SP activity is performed partly by cell membrane-bound neutral endopeptidase CD10 (neprilysin), which is expressed by multiple MSC types. Previous work demonstrated that human IFP-MSC acquire a potent immunomodulatory phenotype and actively degrade SP via CD10 in vitro and in vivo. Importantly, recent data indicated that CD10-bound small extracellular vesicles (sEVs) possess immunomodulatory miRNA attributes with strong immunomodulatory and anabolic effects favoring joint homeostasis in vivo.

In some embodiments, individual MSC sEVs are less than about 200 nanometers in diameter. In some embodiments, individual MSC sEVs are between about 50 nanometers and about 200 nanometers in diameter.

Various CGRP antagonist peptides have been characterized and are suitable for use in the present invention. All CGRP peptides that are CGRP receptor antagonists are contemplated herein.

In some embodiments, the aCGRP comprises a truncated CGRP peptide comprising amino acids 8-37 of CGRP (CGRP8-37).

In some embodiments, CGRP8-37 comprises SEQ ID NO: 1, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, CGRP8-37 comprises the amino acid sequence of SEQ ID NO: 1, or an amino acid sequence at least about 95% identical thereto. In some embodiments, CGRP8-37 comprises the amino acid sequence of SEQ ID NO: 1.

In some embodiments, CGRP8-37 is encoded by a nucleic acid sequence of SEQ ID NO: 2, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, CGRP8-37 is encoded by a nucleic acid sequence of SEQ ID NO: 2, or a nucleic acid sequence at least about 95% identical thereto. In some embodiments, CGRP8-37 is encoded by a nucleic acid sequence of SEQ ID NO: 2.

In some embodiments, the aCGRP comprises a truncated CGRP peptide comprising amino acids 8-37 of β-CGRP (β-CGRP8-37). In some embodiments, β-CGRP8-37 comprises the amino acid sequence of SEQ ID NO: 5, or an amino acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, β-CGRP8-37 comprises the amino acid sequence of SEQ ID NO: 5, or an amino acid sequence at least about 95% identical thereto. In some embodiments, β-CGRP8-37 comprises SEQ ID NO: 5.

In some embodiments, aCGRP is operatively conjugated to a signal sequence. In some embodiments, the signal sequence facilitates the secretion of the aCGRP. Various signal sequence are contemplated herein to facilitate the secretion and maturation of an aCGRP, wherein a specific signal peptide is used depending on the expression system (e.g., mammalian cells, yeast, etc.), and the desired secretion efficiency. In some embodiments, the aCGRP comprises CGRP8-37.

In some embodiments, the signal sequence comprises a peptidylglycine-amidating monooxygenase (ssPAM) signal sequence. A PAM signal sequence, as described herein, is essential for post-translational modification of inactive peptidylglycine precursors to the corresponding bioactive alpha-amidated peptides, a terminal modification in biosynthesis of many neural and endocrine peptides.

In some embodiments, the aCGRP comprises SEQ ID NO: 29, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the aCGRP comprises SEQ ID NO: 29, or a sequence at least about 95% identical thereto. In some embodiments, the aCGRP comprises SEQ ID NO: 29.

In some embodiments, CGRP8-37 comprises SEQ ID NO: 32, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, CGRP8-37 comprises the amino acid sequence of SEQ ID NO: 32, or an amino acid sequence at least about 95% identical thereto. In some embodiments, CGRP8-37 comprises the amino acid sequence of SEQ ID NO: 32.

In some embodiments, the ssPAM signal sequence comprises SEQ ID NO: 3, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM signal sequence comprises the amino acid sequence of SEQ ID NO: 3, or an amino acid sequence at least about 95% identical thereto. In some embodiments, the ssPAM signal sequence comprises the amino acid sequence of SEQ ID NO: 3.

In some embodiments, the ssPAM signal sequence is encoded by a nucleic acid sequence of SEQ ID NO: 4, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM signal sequence is encoded by a nucleic acid sequence of SEQ ID NO: 4, or a nucleic acid sequence at least about 95% identical thereto. In some embodiments, the ssPAM signal sequence is encoded by a nucleic acid sequence of SEQ ID NO: 4.

In some embodiments, the ssPAM-CGRP8-37 fragment amino acid sequence comprises SEQ ID NO: 30, or an amino acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM-CGRP8-37 fragment amino acid sequence comprises SEQ ID NO: 30, or an amino acid sequence at least about 95% identical thereto. In some embodiments, the ssPAM-CGRP8-37 fragment amino acid sequence comprises the amino acid sequence SEQ ID NO: 30.

In some embodiments, the ssPAM-CGRP8-37 fragment is encoded by the nucleic acid sequence of SEQ ID NO: 31, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM-CGRP8-37 fragment is encoded by the nucleic acid sequence of SEQ ID NO: 31, or a nucleic acid sequence at least about 95% identical thereto. In some embodiments, the ssPAM-CGRP8-37 fragment is encoded by the nucleic acid sequence of SEQ ID NO: 31.

In one aspect, provided herein is a viral vector comprising a nucleic acid sequence encoding for a fusion polypeptide as described herein. In some embodiments, the fusion protein comprises aCGRP and an ssPAM signal sequence. In some embodiments, the aCGRP comprises CGRP8-37. In some embodiments, the fusion polypeptide comprises a) a CGRP8-37 peptide comprising SEQ ID NO: 1 or a sequence at least 90% identical to SEQ ID NO: 1, and b) an ssPAM signal sequence comprising SEQ ID NO: 3 or a sequence at least 90% identical to SEQ ID NO: 3. In some embodiments, the fusion polypeptide comprises a) a CGRP8-37 peptide comprising SEQ ID NO: 32 or a sequence at least 90% identical to SEQ ID NO: 32, and b) an ssPAM signal sequence comprising SEQ ID NO: 3 or a sequence at least 90% identical to SEQ ID NO: 3. In some embodiments, the fusion protein comprises SEQ ID NO: 30, or an amino acid sequence at least about 90% identical thereto. In some embodiments the viral vector is a lentiviral vector. In some embodiments, the viral vector is an AAV vector. In some embodiments the AAV vector is AAV2.

In some embodiments, supernatant from the MSC EVs as disclosed herein or from cells transfected with a viral vector as disclosed herein are administered to the subject in need thereof.

Viral vectors are molecular tools used to deliver genetic material into cells that can be performed inside a living organism (in vivo), or in cell culture (in vitro). In some embodiments, the viral vector includes, but is not limited to adeno-associated viral (AAV) vectors, adenoviral vectors, retroviral vectors, lentiviral vectors, and hybrid viral vectors. In some embodiments, the viral vector is selected from a lentiviral vector, an adenoviral vector, or an adeno-associated vector (AAV).

The viral vectors can include a nucleic acid sequence encoding a marker product. The encoded marker product is used to determine if the gene has been delivered to the cell and, once delivered, is being expressed. In some embodiments, the nucleic acid sequence comprises a marker gene comprising E. Coli lacZ gene, which encodes β galactosidase, or a green fluorescent protein encoding gene.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure.

### Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into its nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

In some embodiments, the retroviral vector comprises a retroviral vector.

### Adenoviral Vectors

In some embodiments, the viral vector comprises an adenoviral vector.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

### Adeno-Associated Viral Vectors

In some embodiments, the viral vector comprises an AAV vector. In some embodiments, the AAV vector comprises AAV2.

This defective parvovirus is a preferred vector because it can infect many cell types and is nonpathogenic to humans. AAV type vectors can transport about 4 to 5 kb and wild type AAV is known to stably insert into chromosome 19. Vectors which contain this site-specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

AAV is widely considered the top clinically acceptable candidate due to low toxicity and the ability to provide continued transgene expression and long-term production of desired therapeutic molecules. There have now been over 130 clinical gene therapy trials involving AAVs, with overall excellent safety profile. AAVs providing immunomodulatory cytokine IFNβ, TNFα soluble receptor, or PD-L1 administered via intra-articular injection have been shown in preclinical rheumatoid arthritis models to reduce hindpaw swelling, inflammation, and cartilage damage, and a phase 1/2 clinical trial using rAAV TNF receptor showed good tolerability with modest improvement in joint swelling.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

In some embodiments, the AAV vector comprises an AAV2 serotype. In some embodiments, the AAV vector comprises an AAV9 serotype. In some embodiments, the AAV vector comprises an AAV6 serotype. In some embodiments, the AAV vector comprises an AAV1, AAV2, AAV3, AAV4, AAV5, AAV7, or AAV8 serotype.

In some embodiments, the AAV vector further comprises a woodchuck hepatitis virus posttranscriptional regulatory element (WPRE).

In some embodiments the promoter and/or enhancer region can act as a constitutive promoter and/or enhancer to maximize expression of the region of the transcription unit to be transcribed. In certain constructs, the promoter and/or enhancer region can be active in all eukaryotic cell types, even if it is only expressed in a particular type of cell at a particular time. A preferred promoter of this type is the CMV promoter (650 bases). In some embodiments, the promoter is selected from an SV40 promoter, a cytomegalovirus promoter, a CMV early enhancer/chicken beta actin (CAG) promoter, or a retroviral vector LTR.

In some embodiments, the promoter comprises a cell- or tissue-specific promoter. For example, the glial fibrillary acetic protein (GFAP) promoter has been used to selectively express genes in cells of glial origin. In some embodiments, a heart tissue-specific promoter is used to drive expression in heart tissue (e.g., cardiomyocytes) and is selected from an alpha-Myosin Heavy chain (alpha-MHC) promoter, a myosin light chain 2 (MLC2v) promoter, or a cardiac troponin T (cTnT) promoter. In another aspect, a liver-specific promoter such as an albumin promoter, a thyroxine binding globin promoter, a phosphoenolpyruvatecarboxykinase (PEPCK) promoter, a tyrosine aminotransgerase (TAT) promoter, or a human alpha 1-antitrypsin (hAAT) promoter can be used to drive expression in the liver.

AAV particles are often found with cellular exosomes, small membrane vesicles or microvesicles that are secreted from the cellular membrane into the culture medium. AAV particles are either associated with the outside surface of the exosome or are carried inside the vesicle, which may also contain other cellular molecules such as proteins or nucleic acids. The use of sEVs-enveloped AAVs (sEVs-AAVs), as described herein, can improve transduction, prolong transgene expression, and increase resistance to human neutralizing antibodies compared with standard AAVs. In addition, studies show that EVs enhance AAV entry into cells and directly protect AAV from exogenously administered human neutralizing antibody. This is particularly useful for therapeutic applications in vivo where endogenous anti-AAV antibodies often neutralize the therapeutic efficacy of AAV vectors.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected β-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

In some embodiments, the MSC sEVs are enriched of one or more microRNAs (miRNAs), wherein the mRNAs have anti-inflammatory and/or immunomodulatory effects. “miRNAs” are small non-coding RNAs that regulate gene expression. They exert their effects on the cells they are synthesized in, and are also released into the extracellular space and transported in body fluids such as blood and urine.

In some embodiments, the MSC sEVs are enriched of miRNAs selected from hsa-miR-4466, hsa-miR-3665, hsa-miR-6089, hsa-miR-1290, hsa-miR-4516, hsa-miR-7975, hsa-miR-4454, hsa-miR-21-5p, has-miR-4792, has-miR-4488, has-miR-1246, hsa-miR-23a-3p, hsa-miR- 100-5p, hsa-miR-4286, hsa-miR-99a-5p, has-let-7b-5p, hsa-miR-107, hsa-miR-574-5p, or hsa-miR-27b-3p, or a combination thereof, relative to an RNA normalizer control.

In some embodiments, the MSC sEVs are enriched of anti-inflammatory and/or immunomodulatory miRNAs, relative to an RNA normalizer control. In some embodiments, the anti-inflammatory and/or immunomodulatory miRNAs are selected from hsa-miR-6089, has-miR-21-5p, hsa-miR-23a-3p, hsa-miR-99a-5p, hsa-miR-574-5p, hsa-let-7b-5p, or hsa-miR-1290, or a combination thereof.

In some embodiments, the RNA normalizer control comprises SNORD48.

In some embodiments, the MSC sEVs are enriched of one or more immunomodulatory proteins selected from TIMP-2, IL-8, MCP-1, IL-6, ICAM-1, sTNF-RI, MIP-1β, IL-10, or IP-10, or a combination thereof.

In some embodiments, the MSC sEVs are enriched of one or more reparative proteins selected from HGF, VEGF, EGFR, IGFBP-1, BFGF, or IGFBP-6, or a combination thereof.

In some embodiments, the MSC sEVs are enriched of one or more of Calca, Trpa1, Scn11a, or a combination thereof.

In some embodiments, the MSC sEVs are enriched of one or more long non-coding RNAs (lncRNAs). In some embodiments, the MSC sEVs are enriched of lncRNAs selected from ZFAS1, EMX2OS, HOTAIRM1, RPS6KA2-AS1, DANCR, LINC-ROR, GACAT1, GNAS-AS1, HAR1A, OIP5-AS1, TERC, or a combination thereof.

In another aspect, provided herein is a fusion polypeptide comprising a) a CGRP8-37 peptide, and b) an ssPAM signal sequence.

In some embodiments, the CGRP8-37 peptide comprises SEQ ID NO: 1, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the CGRP8-37 peptide comprises SEQ ID NO: 1, or a sequence at least about 90% identical thereto.

In some embodiments, the CGRP8-37 peptide comprises the amino acid sequence of SEQ ID NO: 1.

In some embodiments, the ssPAM signal sequence comprises SEQ ID NO: 3, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM signal sequence comprises SEQ ID NO: 3, or a sequence at least about 90% identical thereto.

In some embodiments, the ssPAM signal sequence comprises the amino acid sequence of SEQ ID NO: 3.

In some embodiments, the fusion peptide is encoded by a nucleic acid inserted into a viral vector.

In another aspect, provided herein is a viral vector comprising a nucleic acid encoding a calcitonin gene-related antagonist peptide (aCGRP) fusion polypeptide, wherein the fusion polypeptide comprises a) an ssPAM signal sequence, and b) a CGRP8-37 peptide.

In some embodiments, the CGRP8-37 peptide comprises SEQ ID NO: 1, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the CGRP8-37 peptide comprises SEQ ID NO: 1, or a sequence at least about 90% identical thereto. In some embodiments, the CGRP8-37 peptide comprises the amino acid sequence of SEQ ID NO: 1.

In some embodiments, CGRP8-37 is encoded by a nucleic acid sequence of SEQ ID NO: 2, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, CGRP8-37 is encoded by a nucleic acid sequence of SEQ ID NO: 2, or a nucleic acid sequence at least about 90% identical thereto. In some embodiments, CGRP8-37 is encoded by a nucleic acid sequence of SEQ ID NO: 2.

In some embodiments, the ssPAM signal sequence comprises SEQ ID NO: 3, or a sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM signal sequence SEQ ID NO: 3, or a sequence at least about 90% identical thereto. In some embodiments, the ssPAM signal sequence comprises the amino acid sequence of SEQ ID NO: 3.

In some embodiments, the ssPAM signal sequence is encoded by a nucleic acid sequence of SEQ ID NO: 4, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the ssPAM signal sequence is encoded by a nucleic acid sequence of SEQ ID NO: 4, or a nucleic acid sequence at least about 90% identical thereto. In some embodiments, the ssPAM signal sequence is encoded by a nucleic acid sequence of SEQ ID NO: 4.

In some embodiments, the viral vector is selected from a lentiviral vector, an adenoviral vector, or an adeno-associated vector (AAV). In some embodiments, the viral vector comprises a lentiviral vector.

In some embodiments, the lentiviral vector comprises the nucleic acid sequence of SEQ ID NO: 28, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the lentiviral vector comprises the nucleic acid sequence of SEQ ID NO: 28, or a nucleic acid sequence at least about 95% identical thereto. In some embodiments, the lentiviral vector comprises the nucleic acid sequence of SEQ ID NO: 28.

In some embodiments, the viral vector comprises an AAV vector.

In some embodiments, the AAV vector comprises the nucleic acid sequence of SEQ ID NO: 27, or a nucleic acid sequence at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% identical thereto. In some embodiments, the AAV vector comprises the nucleic acid sequence of SEQ ID NO: 27, or a nucleic acid sequence at least about 95% identical thereto. In some embodiments, the AAV vector comprises the nucleic acid sequence of SEQ ID NO: 27.

In some embodiments, the signal peptide sequence is covalently linked to the aCGRP via a linker. Any linker is optional; i.e., any linker may simply be a bond. In some embodiments, the linker serves a spacer function. In some embodiments, the linker comprises from 1 to 30 or less amino acids linked by peptide bonds. In some embodiments, the linker comprises between 1 to 30, between 2 to 25, between 3 and 22, between 5 and 20, between 1 and 10, between 10 and 20, or between 10 and 15 amino acids linked by peptide bonds.

In some embodiments, the amino acids of the linker are selected from the 20 naturally occurring amino acids. In some embodiments, non-natural amino acids are incorporated into the linker either by chemical synthesis, post-translational chemical modification, or by in vivo incorporation by recombinant expression in a host cell. In some embodiments, these amino acids are glycosylated.

In some embodiments, the 1 to 30 or less amino acids are selected from glycine (G), alanine (A), proline (P), asparagine (N), glutamine (Q), lysine (K), aspartate (D), and glutamate (E). In some embodiments, the linker comprises a majority of sterically unhindered amino acids selected from glycine, alanine, serine, or a combination thereof.

In some embodiments, the linker comprises one or more stretches of polyglycines, e.g., GGG, GGGG (SEQ ID NO: 6), GGGGG (SEQ ID NO: 7), of polyalanines, or of polydipeptides selected from (GA)x, (GS)x, (GE)x, (GK)x, (GD)x, or (GR)x motifs, wherein x is an integer of 1, 2, 3, 4, 5, 6, 7, 8, 9, or 10. In some embodiments, the linker is (GGGKGGGG)x (SEQ ID NO: 8); (GGGNGSGG)x (SEQ ID NO: 9); (GGGCGGGG)x (SEQ ID NO: 10); or (GPNGG)x (SEQ ID NO: 11), wherein x is an integer of 1, 2, 3, 4, 5, 6, 7, 8, 9, or 10.

In some embodiments, the linker comprises a combinations of Gly and Ala or combinations of Gly and Ser. In some embodiments, the linker is selected from GGG, (GS)x, (GGS)x, (GGGS)x (SEQ ID NO: 12), or (GGGGS)x (SEQ ID NO: 13), where x is 1, 2, 3, 4, 5, 6, 7, 8, 9, or 10, for example, (GGGS)₁ (SEQ ID NO: 12), (GGGGS)₁ (SEQ ID NO: 13), (GGGS)₄=GGGSGGGSGGGSGGGS (SEQ ID NO: 14), or (GGGGS)₃=GGGGSGGGGSGGGGS (SEQ ID NO: 15).

In some embodiments, the linker comprises an uncharged linker. As used herein, a linker having “uncharged” amino acids is defined as amino acids that do not have an overall charge. Uncharged amino acids include alanine, glycine, valine, leucine, isoleucine, proline, phenylalanine, tryptophan, cysteine, methionine, serine, threonine, asparagine, and glutamine. Uncharged amino acids can be either polar or nonpolar. Polar, uncharged amino acids include serine, threonine, glutamine, and asparagine.

In some embodiments, charged linkers may be used. Charged amino acids include aspartate, glutamate, lysine, serine, and histidine. Such charged linkers may contain a substantial number of acidic residues (e.g., D, E, and the like), or may contain a significant number of basic residues (e.g., K, R, and the like), such that the linker has a pI lower than 7 or greater than 7, respectively. As understood by the person of skill in the art, and all other things being equal, the greater the relative amount of acidic or basic residues in a given linker, the lower or higher, respectively, the pI of the linker will be. Such linkers may impart advantages to the engineered polypeptides disclosed herein, such as improving solubility and/or stability characteristics of such polypeptides at a particular pH, such as a physiological pH (e.g., between pH 7.2 and pH 7.6, inclusive), or a pH of a pharmaceutical composition comprising such polypeptides.

For example, an “acidic linker” is a linker that has a pI of less than 7; between 6 and 7, inclusive; between 5 and 6, inclusive; between 4 and 5, inclusive; between 3 and 4, inclusive; between 2 and 3, inclusive; or between 1 and 2, inclusive. Similarly, a “basic linker” is a linker that has a pI of greater than 7; between 7 and 8, inclusive; between 8 and 9, inclusive; between 9 and 10, inclusive; between 10 and 11, inclusive; between 11 and 12 inclusive, or between 12 and 13, inclusive.

In some embodiments, the acidic linker is selected from (GE)x, (GGE)x, (GGGE)x (SEQ ID NO: 16); (GGGGE)x (SEQ ID NO: 17), (GD)x, (GGD)x, (GGGD)x (SEQ ID NO: 18); (GGGGD)x (SEQ ID NO: 19) wherein x is 1, 2, 3, 4, 5, 6, 7, 8, 9, 10, or more. In some embodiments, the acidic linker is (GGE)₆=GGEGGEGGEGGEGGEGGE (SEQ ID NO: 20).

In some embodiments, the basic linker is selected from (GK)x, (GGK)x, (GGGK)x (SEQ ID NO: 21); (GGGGK)x (SEQ ID NO: 22), (GR)x, (GGR)x, (GGGR)x (SEQ ID NO: 23), or (GGGGR)x (SEQ ID NO: 24) where n is 1, 2, 3, 4, 5, 6, 7, 8, 9, 10, or more. In some embodiments, the linker is (GGR)₅=GGRGGRGGRGGRGGR (SEQ ID NO: 25).

In some additional embodiments, linkers may be prepared which possess certain structural motifs or characteristics, such as an a helix. In some embodiments, a linker may contain an sequence that is selected from the group consisting of (EAAAK)x (SEQ ID NO: 26).

In some embodiments, the linker comprises a non-peptidic linker. In some embodiments, the non-peptide linker is a PEG linker. PEG linkers are known to those of skill in the art.

In some embodiments, the linker is a cleavable linker. Cleavable peptide linkers are known in the art (see, e.g., Alas, M. et al. Peptide-Drug Conjugates with Different Linkers for Cancer Therapy. J Med Chem. 2021 Jan. 14; 64 (1): 216-232).

In some embodiments, the recombinant polypeptides as described herein comprise more than one linker, and each such linker may possess one or more of the characteristics described above.

Molecules can be produced that resemble peptides, but which are not connected via a natural peptide linkage. For example, linkages for amino acids or amino acid analogs can include CH2NH—, —CH2S—, —CH2-CH2-, —CH═CH— (cis and trans), —COCH2-, —CH(OH)CH2-, and —CHHzSO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH2NH—, CH2CH2-); Spatola et al. Life Sci 38:1243-1249 (1986) (—CH H2-S); Hann J. Chem. Soc Perkin Trans. 1307-314 (1982) (—CH—CH—, cis and trans); Almquist et al. J. Med. Chem. 23:1392-1398 (1980) (—COCH2-); Jennings-White et al. Tetrahedron Lett 23:2533 (1982) (—COCH2-). Szelke et al. European Appln, EP 45665 CA (1982): 97:39405 (1982) (—CH(OH)CH2-), Holladay et al. Tetrahedron. Lett 24:4401-4404 (1983) (—C(OH)CH2-); and Hruby Life Sci 31:189-199 (1982) (—CH2-S—), each of which is incorporated herein by reference. A particularly preferred non-peptide linkage is-CH2NH—. It is understood that peptide analogs can have more than one atom between the bond atoms, such as b-alanine, g-aminobutyric acid, and the like.

In one aspect, provided herein is a pharmaceutical composition comprising a population of MSC sEVs as described herein, or a fusion polypeptide as described herein, and a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Methods

In one aspect, provided herein is a method of treating pain in a subject in need thereof comprising administering a therapeutically effective amount of a pharmaceutical composition as described herein. In some embodiments, the pain comprises chronic pain, inflammatory pain, osteoarthritis pain, inflammation, spinal cord injury pain, headache, or neuropathic pain. In some embodiments, the pain comprises chronic pain. In some embodiments, the pain comprises osteoarthritis pain. In some embodiments, the pain comprises neuropathic pain. In some embodiments, the neuropathic pain comprises chronic neuropathic pain. In some embodiments, the pain comprises headache. In some embodiments, the headache comprises migraine or cluster headache. In some embodiments, the pain comprises inflammatory pain. In some embodiments, the pain comprises spinal cord injury pain. In some embodiments, the pain comprises bone fracture pain. In some embodiments, the pain is associated with a disorder selected from IBS, PCOS, Bladder Pain Syndrome, Carpal Tunnel Syndrome, or menstruation- or post-menstruation-related disorders.

In another aspect, provided herein is a method of preventing or treating headache in a subject in need thereof comprising administering a therapeutically effective amount of the pharmaceutical composition as described herein.

In some embodiments, the headache comprises migraine headache or a cluster headache. In some embodiments, the headache comprises migraine headache. In some embodiments, the migraine comprises migraine with aura (MA). In some embodiments, the migraine comprises migraine without aura. In some embodiments, the migraine comprises familial hemiplegic migraine (FHM). In some embodiments, the migraine comprises episodic migraine. In some embodiments, the migraine comprises high frequency episodic migraine and/or chronic migraine. In some embodiments, the migraine is refractory to standard of care treatment.

In some embodiments, the headache comprises post-traumatic headache. In some embodiments, the headache comprises mild traumatic brain injury. In some embodiments, the headache comprises post-concussion syndrome.

In another aspect, provided herein is a method of treating arthritis in a subject in need thereof comprising administering a therapeutically effective amount of the pharmaceutical composition as described herein.

In another aspect, provided herein is a method of treating osteoarthritis (OA) in a subject in need thereof comprising administering a therapeutically effective amount of the pharmaceutical composition as described herein.

In another aspect, provided herein is a method of treating osteoporosis in a subject in need thereof comprising administering a therapeutically effective amount of the pharmaceutical composition as described herein.

In another aspect, provided herein is a method for polarizing macrophages in a subject in need thereof, comprising administering a therapeutically effective amount of the pharmaceutical composition as described herein. In some embodiments, the macrophages are polarized to an anti-inflammatory phenotype. In some embodiments, the macrophages are polarized to an M2 anti-inflammatory phenotype. In some embodiments, the M2 anti-inflammatory phenotype is represented by enrichment of one or more polarization markers selected from CD200R1, BMP7, IRF4, IL10, or IL12A, or a combination thereof.

In another aspect, provided herein is a method for reducing, preventing, or reversing inflammation in a subject in need thereof, comprising administering a therapeutically effective amount of the pharmaceutical composition as described herein.

In another aspect, provided herein is a method for reducing, preventing, or reversing neuroinflammation in a subject in need thereof, comprising administering a therapeutically effective amount of the pharmaceutical composition as described herein.

In another aspect, provided herein is a method of treating joint disease in a subject in need thereof comprising administering a therapeutically effective amount of the pharmaceutical composition as described herein. In some embodiments, the joint disease is characterized by synovitis, IFP fibrosis, articular cartilage degradation, osteoarthritis, or a combination thereof. In another aspect, provided herein is a method of treating neuropathy in a subject in need thereof comprising administering a therapeutically effective amount of the pharmaceutical composition as described herein. In some embodiments, the neuropathy comprises diabetic polyneuropathy. In some embodiments, the neuropathy comprises small fiber neuropathy or fibromyalgia. In some embodiments, the neuropathy comprises Long COVID. In some embodiments, the neuropathy is associated with Type 2 Diabetes. In some embodiments, the neuropathy comprises an ulnar neuropathy. In some embodiments, the neuropathy comprises trigeminal neuropathy.

The therapeutic regimen as described herein may be administered by any route. In some embodiments, the therapeutic regimen is administered via a variety of routes, including intravenous, intraperitoneal, intramuscular, intra-arterial, intramedullary, intrathecal, subcutaneous, intraventricular, transdermal, interdermal, mucosal, nasal, oral, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the therapeutic regimen (e.g., its stability in the environment of the body), the condition of the subject (e.g., whether the subject is able to tolerate administration by injection), etc. In some embodiments, the therapeutic regimen of any preceding aspect can be administered by in vivo electroporation. As used herein, “in vivo electroporation” refers to a physical method of gene transfer, wherein a series of electric or mechanical pulses are delivered through a subject's intact tissue or cell for a short duration of time to enhance the transfer of macromolecules, including but not limited to DNA, RNA, and proteins.

In some embodiments, the pharmaceutical composition is administered via a targeted injection route. In some embodiments, the pharmaceutical composition is injected via subcutaneous, intravenous, intramuscular, intra-articular, intra-synovial, intrasternal, intrathecal, intra-DRG, intrahepatic, intralesional, and intracranial injections or infusion techniques. In some embodiments, the injection is in the trigeminal (gasserian) ganglion.

In some embodiments, the pharmaceutical composition is administered via a targeted intrathecal, intra-ganglionic, intra-spinal, or intra-articular injection.

In some embodiments, the pharmaceutical composition is administered locally via intravitreal, intracochlear, or intra-cerebral injection.

In some embodiments, the pharmaceutical composition (including, for example, the IFP-MSC derived sEVs comprising AAV-aCGRP as described herein) is administered in an amount of 0.001 to 1000 mg/day. In embodiments, the composition is administered in a range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg).

The exact amount of the therapeutic regimen required to achieve a therapeutically effective amount will vary from subject to subject, depending on species, age, weight, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the pharmaceutical composition is administered in a single injection.

In some embodiments, the pharmaceutical composition is administered in more than one injection. In some embodiments, subsequent administrations of the pharmaceutical composition are administered once every other day (Q2D), once every three days (Q3D), once every 4 days (Q4D), once per week (Q1W), once every 2 weeks (Q2W), once every three weeks (Q3W), once every 4 weeks (Q4W), once monthly, twice per month, once every other month, once every 6-months, once per year, or a combination thereof.

In some embodiments, the pharmaceutical composition is administered in combination with one or more analgesics. In some embodiments, the one or more analgesics are selected from acetaminophen, aspirin, caffeine, or ibuprofen. In some embodiments, the one or more analgesics are selected from an anti-migraine agent, a Cox-2 inhibitor, a nonsteroidal anti-inflammatory drug (NSAID), or a salicylate.

In some embodiments, the anti-migraine agent is selected from almotriptan (AXERT®), dihydroergotamine nasal (Migranal®), eletriptan (RELPAX®), ergotamine (Ergomar®), ergotamine+caffeine (Cafergot; Migergot®), frovatriptan (Frova), naratriptan (AMERGER), rizatriptan (MAXALT®, Maxalt-MLT), sumatriptan injection (Imitrex, Imitrex Nasal), sumatriptan+naproxen (TREXIMET®), or zolmitriptan (ZOMIG®, Zomig-ZMT).

In some embodiments, the one or more analgesics comprise acetaminophen (Actamin, Mapap, TYLENOL®).

In some embodiments, the Cox-2 inhibitor comprises celecoxib (CELEBREX®).

In some embodiments, the NSAID is selected from flurbiprofen (ANSAID®), ketorolac (nasal) (SPRIX®), ketoprofen (ORUVAIL SR®), tolmetin (TOLECTIN® DS), ibuprofen (Advil, MOTRIN®, Genpril, Midol IB, Proprinal), naproxen (Aleve®, Flanax Pain Reliever, Midol Extended Release, Naprosyn), indomethacin (Indocin®, Tivorbex®), sulindac (CLINORIL®), piroxicam (FELDENE®), mefenamic acid (Ponstel), meloxicam (MOBIC®, VIVLODEX), diclofenac (Cataflam®, Voltaren, ZIPSOR®, ZORVOLEX), celecoxib (CELEBREX®), etodolac (Lodine®), etoricoxib, lumiracoxib, or rofecoxib.

In some embodiments, the salicylate is selected from aspirin, diflunisal (DOLOBID®), magnesium salicylate, or salsalate (Disalcid).

In some embodiments, the one or more analgesics are administered at a lower dose relative to the standard of care when administered in combination with a pharmaceutical composition as described herein.

In some embodiments, the one or more analgesics are administered less frequently relative to the standard of care when administered in combination with a pharmaceutical composition as described herein.

In some embodiments, the subject is a human.

In an alternative aspect, provided herein is a method of obtaining a population of mesenchymal stem/stromal cell-derived small extracellular vesicles (MSC sEVs) comprising a viral vector encoding a calcitonin gene-related antagonist peptide (aCGRP), comprising: (i) obtaining a tissue sample of an infrapatellar fat pad (IFP) of a subject; (ii) mechanically dissecting the IFP tissue sample; (iii) washing the dissected IFP tissue sample in the presence of one or more digestion enzymes in a buffered solution; (iv) inactivating the one or more digestion enzymes; (v) culturing the digested IFP tissue sample in media to produce a population of IFP-MSCs; (vi) transducing the population of IFP-MSCs with one or more viral vectors encoding a CGRP8-37; and (vii) performing one or more purification steps to isolate the population of MSC sEVs, wherein the one or more purification steps are selected from filtering, centrifuging, or a combination thereof. In some embodiments, the aCGRP comprises a truncated CGRP peptide comprising amino acids 8-37 of CGRP (CGRP8-37). In some embodiments, the viral vector comprises an AAV vector.

In some embodiments, the MSC sEVs collection method used is based on centrifugal force to remove other particles (e.g., cell debris), and is at least 80%, 85%, 90%, 95%, 96%, 97%, 98%, or 99% pure. The extracellular vesicles can also be substantially purified. The term “substantially purified” as used herein may refer to extracellular vesicles or exosomes that are substantially enriched in a sample. The sample can be substantially purified or enriched for the extracellular vesicles or exosomes of interest such that the sample is at least about 50%, 60%, 70%, 80%, 85%, 90%, 95%, 99% or greater of the desired extracellular vesicles or exosomes or less than about 40%, 30%, 20%, 15%, 14%, 13%, 12%, 11%, 10%, 9%, 8%, 7%, 6%, 5%, 4%, 3%, 2%, 1% or less of the undesirable or other extracellular vesicles or exosomes present. In an exemplary embodiment, a composition includes substantially purified extracellular vesicle. In other embodiments, the composition includes a substantially purified exosome. In other examples, the composition includes a substantially purified glial-derived extracellular vesicle and at least one substantially purified glial-derived exosome. Another embodiment is directed to a pharmaceutical composition for treating or preventing a neuropathy in a subject including a therapeutically effective amount of substantially purified glial-derived extracellular vesicle or a glial-derived exosome.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with a neuropathy, and compared to samples from known conditions, e.g., a subject (or subjects) that does not have a neuropathy (a negative or normal control), or a subject (or subjects) who does have a neuropathy (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

In another aspect, provided herein is a method of treating pain in a subject in need thereof, comprising administering to the subject a population of isolated MSC sEV obtained by a method of culturing and isolating as described herein.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Modification of Mesenchymal Stem/Stromal Cell-Derived Small Extracellular Vesicles by Calcitonin Gene Related Peptide (CGRP) Antagonist

During the progression of knee osteoarthritis (OA), the synovium and infrapatellar fat pad (IFP) can serve as a source for Substance P (SP) and calcitonin gene-related peptide (CGRP), two important pain-transmitting, immune, and inflammation modulating neuropeptides. Previous studies showed that infrapatellar fat pad-derived mesenchymal stem/stromal cells (MSC) acquire a potent immunomodulatory phenotype and actively degrade Substance P via CD10 both in vitro and in vivo.

It was hypothesized that CD10-bound IFP-MSC sEVs can be engineered to target CGRP while retaining their anti-inflammatory phenotype.

### Materials and Methods

### Isolation, Culture and Expansion of IFP-MSC

All experiments using human cells were performed in accordance with the relevant guidelines and regulations. Human IFP-MSC were isolated from IFP tissue obtained from de-identified, non-arthritic patients [n=5; two males 26 and 48 years old (labelled as MSC1 and MSC2), and three females 22, 42, 44 years old (labelled as MSC3, MSC4, MSC5)] undergoing elective knee arthroscopy. IFP tissue (5-10 cc) was mechanically dissected and washed repeatedly with Dulbecco's Phosphate Buffered Saline (DPBS; Sigma-Aldrich, St Louis, MO, USA), followed by enzymatic digestion using 235 U/mL Collagenase I (Worthington Industries, Columbus, OH, USA) diluted in DPBS and 1% bovine serum albumin (Sigma) for 2 h at 37° C. with agitation. Enzymatic digestion was inactivated with complete media with DMEM low glucose (1 g/L) GlutaMAX (ThermoFisher Scientific, Waltham, MA, USA) containing 10% fetal bovine serum (FBS; VWR, Radnor, PA, USA), washed and seeded at a density of 1×10⁶ cells/175 cm² flask in chemically reinforced (Ch-R) medium. Complete Ch-R medium was prepared by mixing Mesenchymal Stem Cell Growth Medium 2 with supplement provided according to manufacturer's instructions (PromoCell, Heidelberg, Germany). At 48 h post-seeding, non-adherent cells were removed by DPBS rinsing and fresh media were replenished accordingly. MSC were cultured at 37° C., 5% (v/v) CO2 until 80% confluent as passage 0 (P0), then passaged at a 1:5 ratio until P3, detaching them with TrypLE™ Select Enzyme 1× (Gibco, ThermoFisher Scientific) and assessing cell viability with 0.4% (w/v) Trypan Blue (Invitrogen, ThermoFisher Scientific).

### Generation of AAV Vector Containing GFP-Labeled CGRP Antagonist Gene

CGRP8-37 is a truncated polypeptide of CGRP that serves as a competitive antagonist of CGRP (aCGRP). To create a gene construct, human CGRP cDNA was purchased from (Open Biosystems, ThermoFisher Scientific). The PCR fragment was ligated to pGEMT-Easy cloning vector and CGRP8-37 fragment subcloned downstream of peptidylglycine-amidating monooxygenase (ssPAM) signal sequence to allow CGRP8-37 to be amidated and secreted. ClaI-BamHI fragment was subcloned into pAAV-EGFP-WPRE viral vector. The ssPAM-CGRP8-37 was subcloned to AAV-EGFP-WPRE and lenti-EGFP-WPRE vectors and viral particles were developed.

IFP-MSC Transduction and Cell Sorting for GFP Positive aCGRP IFP-MSC

AAV-aCGRP-GFP was used to transduct 50×10⁶ P1 IFP-MSC (n=5). Transducted cultures were maintained at 37° C., 5% (v/v) CO2 for 7 days and GFP fluorescence was visualized using ×10 objective Leica DMi8 microscope with Leica LAS X software (Leica). On day 7, the transducted IFP-MSC were sorted based on GFP expression to yield the aCGRP IFP-MSC subpopulation using MoFlo Astrios EQ cell sorter (Beckman Coulter, Brea, CA, USA). After sorting, aCGRP IFP-MSC were seeded on 0.1% gelatin plates (MilliporeSigma, St Louis, MO, USA) and were cultured at 37° C., 5% (v/v) CO2 until 80% confluency.

Clonogenic Assay of aCGRP IFP-MSC

aCGRP IFP-MSC at passage three (P3) (n=3) were seeded in 25 cm² flasks in triplicate at a density of 5×10² cells per flask in complete medium. Colony-forming unit fibroblasts (CFU-Fs; consisting of at least 50 cells each) were manually enumerated on day 15 after cytochemical staining with 0.01% Crystal Violet (MilliporeSigma, Billerica, MA, USA).

Immunophenotype of aCGRP IFP-MSC

Flow cytometric analysis was performed on P3 aCGRP IFP-MSC (n=3). Briefly, 2× 105 cells were labelled with antibodies specific for: CD10, CD73, CD90, CD105 (Biolegend, San Diego, CA, USA), CD146 (Miltenyi Biotec, Auburn, CA, USA), HLA-DR (BD Biosciences, San Jose, CA, USA) and the corresponding isotype controls. All cells were stained with eFluor 780 fixable viability dye (Invitrogen). The fluorescent signal was acquired using a CytoFLEX S (20,000 events) and analyzed with Kaluza analysis software (Beckman Coulter).

Isolating sEVs from aCGRP IFP-MSC sEVs were isolated from aCGRP IFP-MSC conditioned media by a stepwise ultracentrifugation method. Briefly, conditioned media from aCGRP IFP-MSC cultured in sEVs-depleted Ch-R medium are filtered through a 0.22 μm filter to remove debris and large vesicles, and differentially centrifuged for 2000×g for 10 min and ultracentrifuged for 120,000×g for 4 h using SW 40 Ti Swinging-Bucket Rotor for Optima XPN ultracentrifuge (Beckman Coulter). Pre-enriched sEVs preparations were stored in DPBS at −80° C. Samples from each group were assessed for biophysical and biochemical characterization using flow cytometry and nanoparticle tracking analysis.

Pre-enriched sEVs were incubated with the Dynabeads®-based Exosomes-Human CD63 Isolation/Detection Reagent (Invitrogen) and purified according to the manufacturer's instructions for magnetic selection. CD9 (Invitrogen) expression was used to validate sEVs presence in CD63+-gated particles by flow cytometry. The specific fluorescent labeling of 20,000 events was analyzed on a CytoFLEX S with Kaluza 2.2.1 analysis software (Beckman Coulter).

Nanoparticle tracking analysis (NTA) (NanoSight NS300, Malvern, Worcestershire, UK) from aCGRP IFP-MSC sEVs was performed for quantity and size determination. All samples were diluted 1:10 in PBS. The following settings were set according to the manufacturer's software: detection threshold 5; room temperature; number of frames 30 and measurement time 30 s. The size distribution and particle concentration each represent the mean of five individual measurements.

The functional assessment of aCGRP IFP-MSC sEVs by macrophage polarization and cortical neurons neuroinflammation assays was performed in a concentration corresponding to sEVs secreted from 1×10⁶ aCGRP IFP-MSC.

miRNA Profile of aCGRP IFP-MSC sEVs

miRNA was extracted from aCGRP IFP-MSC sEVs using Total Exosome RNA and Protein Isolation Kit (Thermo Fisher Scientific) according to manufacturer's instructions. Total sEVs miRNA (1 μg) was used for first-strand cDNA synthesis with All-in-One miRNA First-Strand cDNA Synthesis Kit (GeneCopoeia, Rockville, MD, USA).

Pre-designed human MSC exosome 166 miRNA qPCR arrays (GeneCopoeia) (Table 1) were performed using 1000 ng cDNA per IFP-MSC sample (n=2), and processed using StepOne Real-time thermocycler (Applied Biosystems, LLC, Waltham, MA, USA). Data analysis was performed using qPCR results with GeneCopoeia's online Data Analysis System (accessed on 10 Jan. 2024). Mean values were normalized to small nucleolar RNA, C/D box 48 (SNORD48), expression levels were calculated using the 2-ΔCt method. Putative miRNA interactomes were generated using a miRNet centric network visual analytics platform (accessed on 20 January 2024). The miRNA target gene data were collected from well-annotated database miRTarBase v8.0 and miRNA-gene interactome network refining was performed with 2.0 betweenness cut-off. Values (with 34 cycles cut-off point) were represented in a topology miRNA-gene interactome network using force atlas layout and hypergeometric test algorithm. miRDB online database (accessed on 22 Jan. 2024) for prediction of functional miRNA targets has been used to correlate highly expressed target genes in macrophages and synoviocytes with specific miRNAs identified by IFP-MSC sEV miRNA profiling. MirTarget prediction scores are in the range of 0-100% probability, and candidate transcripts with scores ≥50% are presented as predicted miRNA targets in miRDB.

Protein Profile of aCGRP IFP-MSC sEVs

Multiplex protein arrays of 35 cytokines and 41 growth factors (RayBio® C-Series, RayBiotech, Peachtree Corners, GA) (Table 2) were used to determine aCGRP IFP-MSC SEV protein cargos (n=2). Specifically, 132 μg/mL of aCGRP IFP-MSC sEV protein extract was used for each assay following the manufacturer's instructions. Data shown represent 30 sec exposure in FluorChem E chemiluminescence imaging system (ProteinSimple, San Jose, CA, USA). Results were generated by quantifying the mean spot pixel density of each array using protein array analyzer plugin using ImageJ Fiji software (Fiji/ImageJ, NIH website). Results generated by ImageJ Fiji software were introduced into excel-based analysis software tools for the automatic computation of the extracted numerical data obtained from the array image (accessed on 10 Feb. 2024). All signal intensities were normalized with the background signal of each array. Also, all signal intensities were normalized to positive and negative signal to reduce batch variability. Finally, the quantified signal intensity for each protein spot is proportional to the relative concentration of the antigen in the sample.

### Pathway Analysis

Putative interactomes were generated by the Search Tool for Retrieval of Interacting Genes/Proteins (STRING 11.0; accessed on 20 Feb. 2024) database using interaction data from experiments, databases, neighborhoods in genome, gene fusions, co-occurrence across genomes, co-expression and text-mining. An interaction confidence score of 0.4 was imposed to ensure high interaction probability. K-means clustering algorithm was used to organize proteins into 3 separate clusters per condition tested, discriminated by colors. Functional enrichments related to biological process, Kyoto Encyclopedia of Genes and Genomes (KEGG) pathways, and reactome pathways were presented in radar graphs for proteins tested.

The miRDB online database (accessed on 22 Feb. 2024) for prediction of functional miRNA targets has been used to correlate highly expressed target genes in macrophages with specific miRNAs identified by aCGRP IFP-MSC sEV miRNA profiling. MirTarget prediction scores are in the range of 0-100% probability, and candidate transcripts with scores ≥50% are presented as predicted miRNA targets in miRDB.

Culturing HEK 293 with aCGRP IFP-MSC sEVs

HEK293 cells were cultured in growth medium (DMEM/10% FBS/1% PenStrep) and upon 80% confluency were detached, centrifuged and resuspended in 1 mL of growth medium with the addition of aCGRP IFP-MSC sEVs at 30 μg/mL, 50 μg/mL and 80 μg/mL concentrations. Cells/aCGRP IFP-MSC sEVs were incubated at 37° C., 5% (v/v) CO2 for 1 h and then HEK293 cells were re-cultured for 5-7 days. From this culture, 5×10⁴ cells were seeded per well of 12-well plate and cultured overnight. The next day, cells were fixed with 4% paraformaldehyde and incubated in 5% blocking serum for 2 h, followed by overnight incubation with primary antibodies against CGRP (1:500, Abcam) and AAV proteins (1:500, Invitrogen). Finally, cells were washed with 0.1M PBS, incubated in fluorescent secondary antibody (1:250, AlexaFluor, Invitrogen) with DAPI as a nuclear marker, and coverslipped with Vectastain (VectorLabs, Newark, USA). Images were evaluated under 20× objective with confocal microscope (Dragonfly, Andor, South Windsor, USA), digitalized, and analyzed with Image J Fiji software (NIH). To measure and compare the optical density of the CGRP staining between different treatments, at least 3 sections per slide with at least 30 DAPI positive profiles were analyzed. Each section was loaded into Image J Fiji software under CGRP filter, thresholded to the dimmest pixels and the overall integrated density of the section was measured.

### Macrophage Polarization Assay

Human monocytes (THP-1, ATCC) were differentiated into macrophages using PMA/IO (Phorbol 12-myristate 13-acetate/Ionomycin) and polarized to M1 macrophages by M1-macrophage generation medium (PromoCell, Heidelberg, Germany). 5×10⁴ PMA/IO-stimulated THP-1 (macrophages) were mixed with aCGRP IFP-MSC sEVs (n=2) per well of 24-well plate and cultured in M1-macrophage generation medium for 2 days. Macrophage polarization status was assessed using a polarization qPCR array (ScienCell, Carlsbad, CA, USA).

RNA extraction from THP-1 cultures was performed using the RNeasy Mini Kit (Qiagen, Frederick, MD, USA) according to manufacturer's instructions. Total RNA (1 μg) was used for reverse transcription with a SuperScript™ VILO™ cDNA synthesis kit (Invitrogen). A pre-designed 40 gene Human Macrophage Polarization array (GeneQuery™ Human Macrophage Polarization Marker qPCR Array Kit, ScienCell) (Table 3) was performed using 1000 ng cDNA per culture and processed using a StepOne Real-time thermocycler (Applied Biosystems, LLC). Mean values were normalized to GAPDH, expression levels were calculated using the 2-ΔΔCt method. Values were represented in a stacked bar plot for M0, M1, and M2 polarization as the relative fold change of the PMA/IO+THP-1/aCGRP IFP-MSC sEVs to PMA/IO+THP-1 (reference sample, 2-ΔΔCt=X sample/X reference sample).

### Cortical Neurons Neuroinflammation Assay

E14 rat cortical neurons were isolated from rat brain according to protocol. 1×10⁵ TIC inflammatory/fibrotic cocktail (15 ng/ml TNFα, 10 ng/mL IFNγ, 10 ng/mL CTGF)-stimulated cortical neurons were mixed with aCGRP IFP-MSC sEVs (n=2) per well of 12-well plate and cultured for 3 days. Cortical neurons neuroinflammation status was assessed using a neuropathic and inflammatory qPCR array (Qiagen).

RNA extraction from TIC-stimulated cortical neurons was performed using the RNeasy Mini Kit (Qiagen) according to manufacturer's instructions. Total RNA (1 μg) was used for reverse transcription with a SuperScript™ VILO™ cDNA synthesis kit (Invitrogen). A pre-designed 84 gene rat neuropathic and inflammatory array (RT2 Profiler neuropathic & inflammatory array, Qiagen) (Table 4) was performed using 1000 ng cDNA per culture and processed using a StepOne Real-time thermocycler (Applied Biosystems, LLC). Mean values were normalized to ACTB, expression levels were calculated using the 2-ΔΔCt method. Values were represented in a stacked bar plot as the relative fold change of the TIC-stimulated cortical neurons/aCGRP IFP-MSC SEVs to TIC-stimulated cortical neurons alone (reference sample, 2-ΔΔCt=X sample/X reference sample).

### Statistical Analysis

Normal distribution of values was assessed by the Kolmogorov-Smirnov normality test. In the presence of a non-normal distribution of the data, one-way or two-way ANOVA were used for multiple comparisons. All tests were performed with GraphPad Prism v7.03 (GraphPad Software, San Diego, CA, USA). Statistical significance was considered p<0.05.

### Results

aCGRP IFP-MSC Generation and Characterization

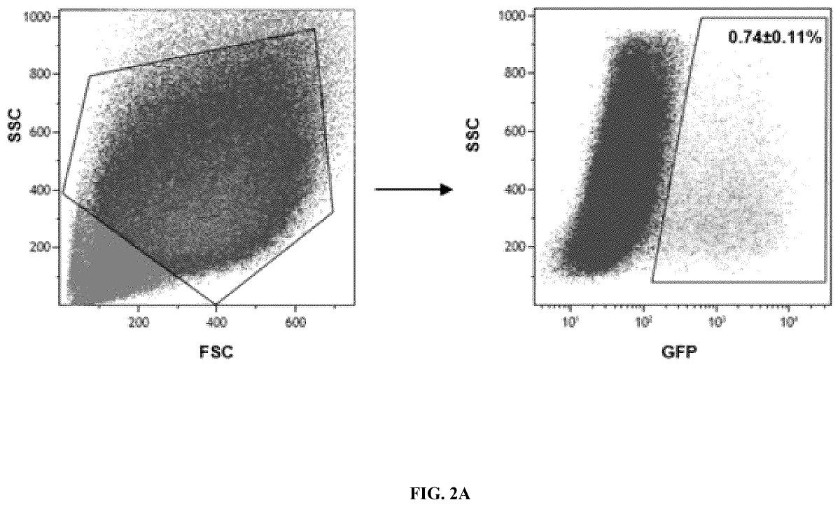

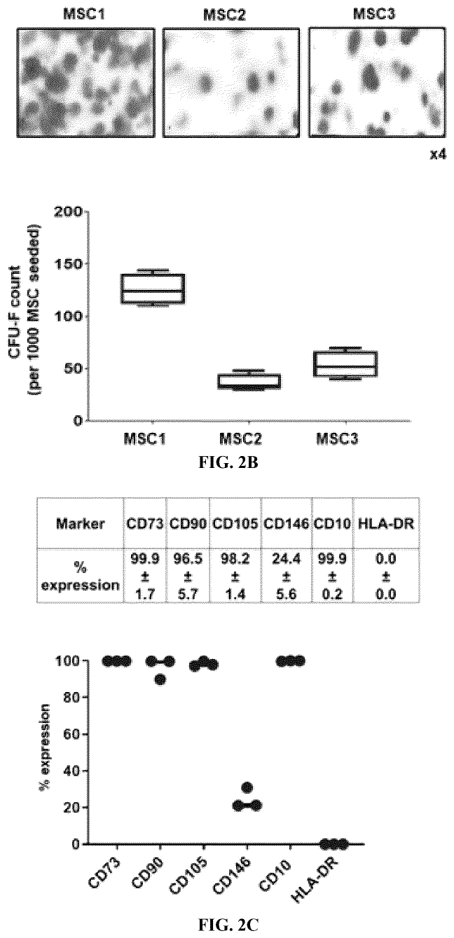

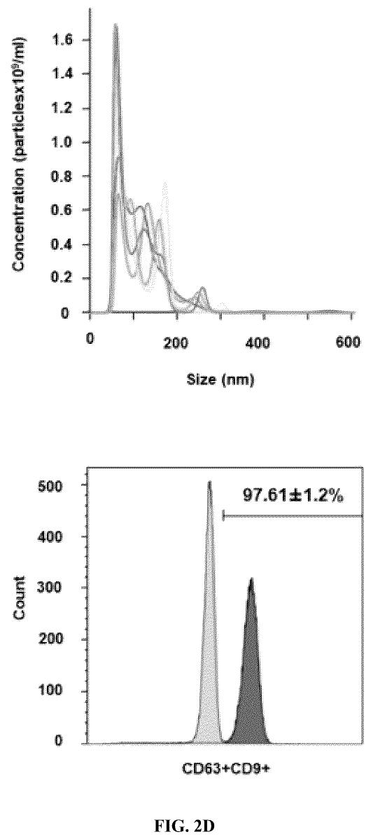

Cultured IFP-MSC was successfully transduced in regulatory-compliant medium with GFP-labelled AAV CGRP8-37 which were maintained for 7 days in vitro until confluency. Cell sorting of transduced cells resulted in purification of an aCGRP IFP-MSC subpopulation (0.74±0.11%, FIGS. 2A-2E).

The present application investigates the effects of AAV CGRP8-37 transduction on IFP-MSC basic characteristics. Therefore, following positive selection, aCGRP IFP-MSC were characterized by standard assays, showing similar fibroblast-like morphology, lower clonogenic capacity (72±42 CFU-Fs) and similar immunophenotyping profiles to non-transduced IFP-MSC published in previous studies. Specifically, transduction with AAV CGRP8-37 had a significant impact on aCGRP IFP-MSC clonogenicity whereas aCGRP IFP-MSC showed high (>90%) expression for common MSC-defining markers (FIG. 2B-2C). CD10 expression was similarly high (>95%), whereas CD146 showed reduced expression compared to non-transduced IFP-MSC (24.4±5.6% vs. 75.4±4.5%). Herein, high CD10 expression levels indicate that aCGRP IFP-MSC show increased functionality in SP degradation. Finally, HLA-DR expression was absent indicating that transduction with AAV CGRP8-37 is not affecting the non-immunogenic profile of IFP-MSC.

aCGRP IFP-MSC sEVs showed high purity with <200 nm sizes, a concentration of 3-4×10⁹/mL, and high expression levels of CD9 marker upon ultracentrifugation (111.7±4.3 nm, FIG. 2D). Immunostaining analysis of HEK293 cells incubated with aCGRP IFP-MSC sEVs showed a positive signal for AAV proteins and CGRP protein, suggesting successful transfer of AAV CGRP8-37 within sEVs (FIG. 2E). Immunodensity of CGRP signal was proportional to the total protein content of sEVs used for transfer. The protein concentrations used 30, 50, 80 μg/mL are corresponding to sEVs secreted from 0.3×10⁶, 0.5×10⁶, and 0.8×10⁶ aCGRP IFP-MSC, respectively. Lower values of AAV integrated density but enhanced CGRP concentration were observed at 80 μg/mL total protein of sEVs compared to 50 μg/mL total protein.

aCGRP IFP-MSC sEVs miRNA Cargo Characterization

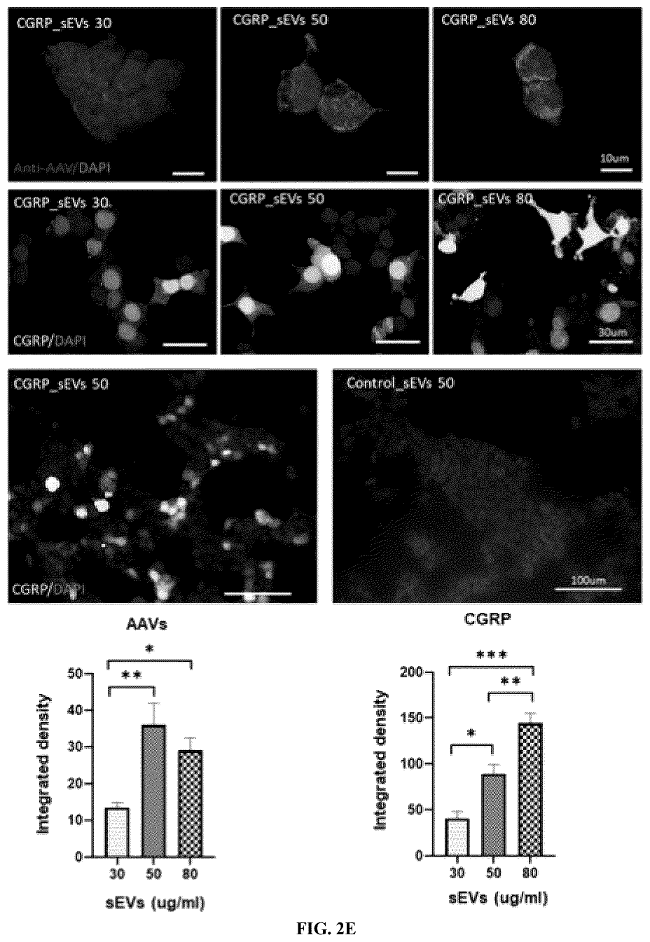

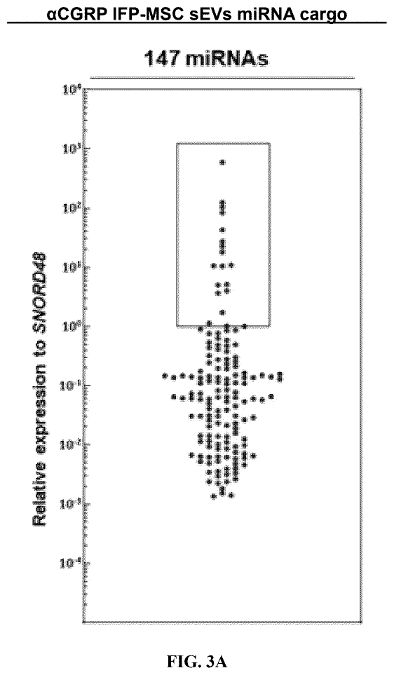

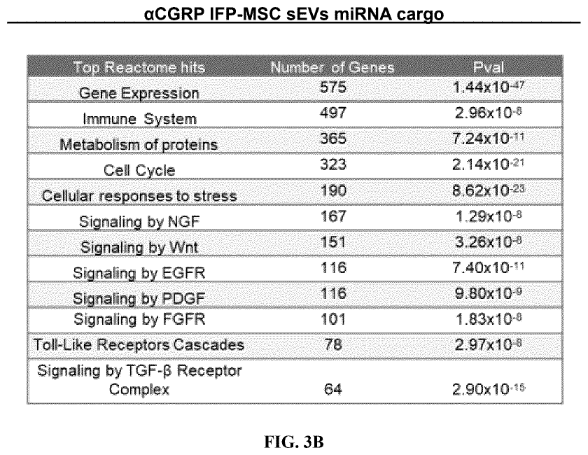

From 166 MSC-related miRNAs analyzed, 147 miRNA cargos were present in aCGRP IFP-MSC sEVs (FIG. 3A). Reactome analysis of the miRNAs present in sEVs showed their involvement in the regulation of mainly six gene groups in target cells related to gene expression, immune system, TGF-β/Wnt/FGFR pathways, cell cycle, NGF and Toll-like receptor pathways, and cellular responses to stress (FIG. 3B). Out of the overall 7457 genes in target cells regulated by miRNAs present in sEVs, 497 genes were related to the immune system, 316 genes were related to TGF-β/Wnt/FGFR pathways, and 245 genes were related to NGF and Toll-like receptor pathways. These results indicate the high involvement of aCGRP IFP-MSC sEVs miRNA cargo in the regulation of a plethora of pathways related to immunomodulation and pain signaling.

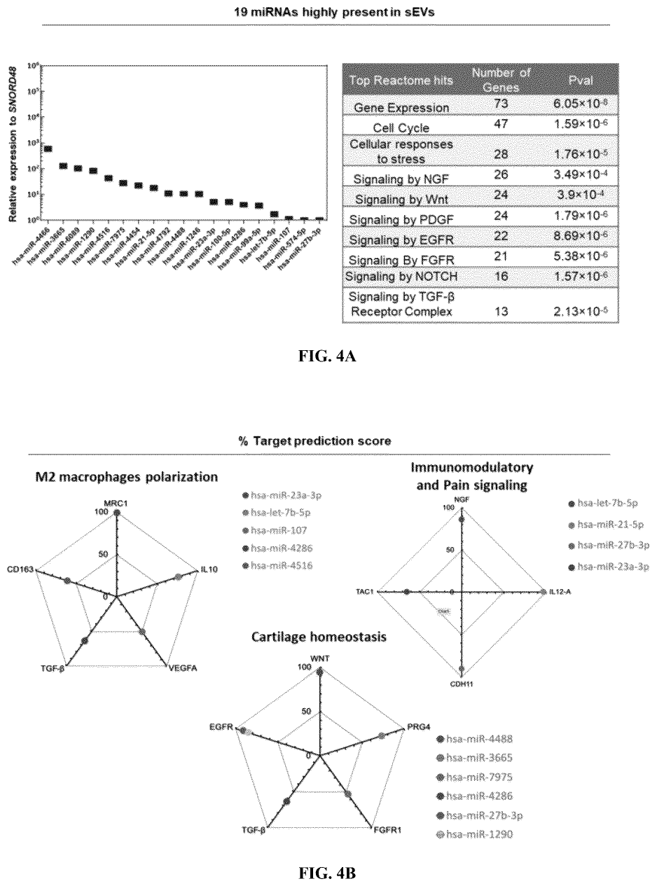

In aCGRP IFP-MSC sEVs, 19 miRNAs cargos were highly present (hsa-miR-4466, hsa-miR-3665, hsa-miR-6089, hsa-miR-1290, hsa-miR-4516, hsa-miR-7975, hsa-miR-4454, hsa-miR-21-5p, has-miR-4792, has-miR-4488, has-miR-1246, hsa-miR-23a-3p, hsa-miR-100-5p, hsa-miR-4286, hsa-miR-99a-5p, has-let-7b-5p, hsa-miR-107, hsa-miR-574-5p, and hsa-miR-27b-3p) (FIG. 4A). From these miRNAs, 7 miRNAs (hsa-miR-6089, has-miR-21-5p, hsa-miR-23a-3p, hsa-miR-99a-5p, hsa-miR-574-5p, hsa-let-7b-5p, hsa-miR-1290) were associated in previous studies with significant anti-inflammatory/immunomodulatory effects in vitro and in vivo. Reactome analysis of 19 miRNAs highly present in aCGRP IFP-MSC sEVs showed their involvement in the regulation of four gene groups in target cells related to the: gene expression, TGF-β/Wnt/FGFR signaling pathways, cell cycle, and NGF signaling (FIG. 4A). Therefore, these distinct miRNAs regulate genes involved in the production of pro-inflammatory cytokines, the recruitment of monocytes, and cartilage homeostasis.

The miRDB in silico analysis revealed a functional correlation of identified miRNAs in aCGRP IFP-MSC sEVs with genes involved in M2 macrophage polarization, immunomodulatory and pain signaling, and cartilage homeostasis (FIG. 4B). For M2 macrophage polarization the MirTarget prediction score system revealed MRC1 as a target for hsa-miR-23a-3p (99% probability), IL10 as a target for hsa-let-7b-5p (76% probability), VEGFA as a target for hsa-miR-107 (51% probability), TGF-β as a target for hsa-miR-4286 (64% probability), and CD163 as a target for hsa-miR-4516 (61% probability). For immunomodulatory and pain signaling the prediction scoring algorithm revealed NGF as a target for hsa-let-7b-5p (86% probability), IL12A as a target for hsa-miR-21-5p (97% probability), CDH11 as a target for hsa-miR-27b-3p (90% probability), and TAC1 as a target for hsa-miR-23a-3p (65% probability). Lastly, for cartilage homeostasis the prediction scoring system revealed WNT as a target for hsa-miR-4488 (94% probability), PRG4 as a target for hsa-miR-3665 (73% probability), FGFR1 as target for hsa-miR-7975 (54% probability), TGF-β as a target for hsa-miR-4286 (64% probability), and EGFR as a target for both hsa-miR-27b-3p (91% probability) and hsa-miR-1290 (85% probability). Overall, from a clinical standpoint, the potent functionality of these miRNAs supports the notion of developing novel cell-free therapeutics for inflammation/pain reversal based on aCGRP IFP-MSC SEVs.

aCGRP IFP-MSC sEVs Protein Cargo Characterization

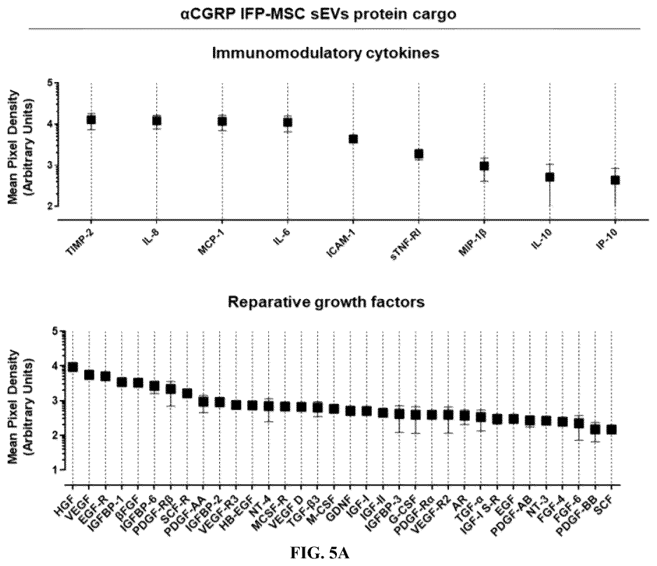

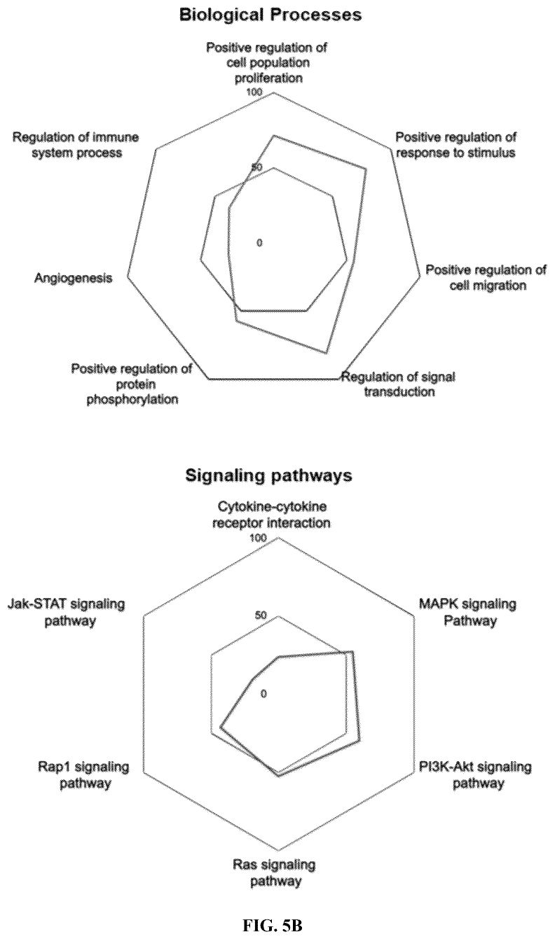

Multiple immunomodulatory and reparative molecules were detected as secreted as a cargo of aCGRP IFP-MSC sEVs (FIG. 5A). sEVs showed presence of key immunomodulatory molecules including TIMP-2, IL-8, MCP-1, IL-6, ICAM-1, sTNF-RI, MIP-1β, IL-10, and IP-10. In parallel, key reparative molecules including HGF, VEGF, EGFR, IGFBP-1, BFGF, and IGFBP-6 showed presence in aCGRP IFP-MSC sEVs.

All proteins appeared interconnected at least through one association and all K-means clustering networks demonstrated elevated protein-protein interaction enrichment (p<1×10-16) and an average local clustering coefficient >0.8 indicating that the proteins used are at least partially biologically connected. In terms of biological processes, various categories were highly affected and presented as % of proteins involved in a category to the total proteins detected. Specifically, regulation of signal transduction (81%), positive regulation of response to stimulus (79%), positive regulation of cell population proliferation (71%), positive regulation of cell migration (55%), positive regulation of protein phosphorylation (57%), regulation of immune system process (38%), and angiogenesis (31%) (FIG. 5B). Regarding the type of signaling pathways affected, aCGRP IFP-MSC sEVs have profound effects on PI3K-Akt signaling pathway (59%), MAPK signaling pathway (55%), Ras signaling pathway (52%), Rap1 signaling pathway (43%), cytokine-cytokine receptor interaction (24%), and Jak-STAT signaling pathway (19%) (FIG. 5B).

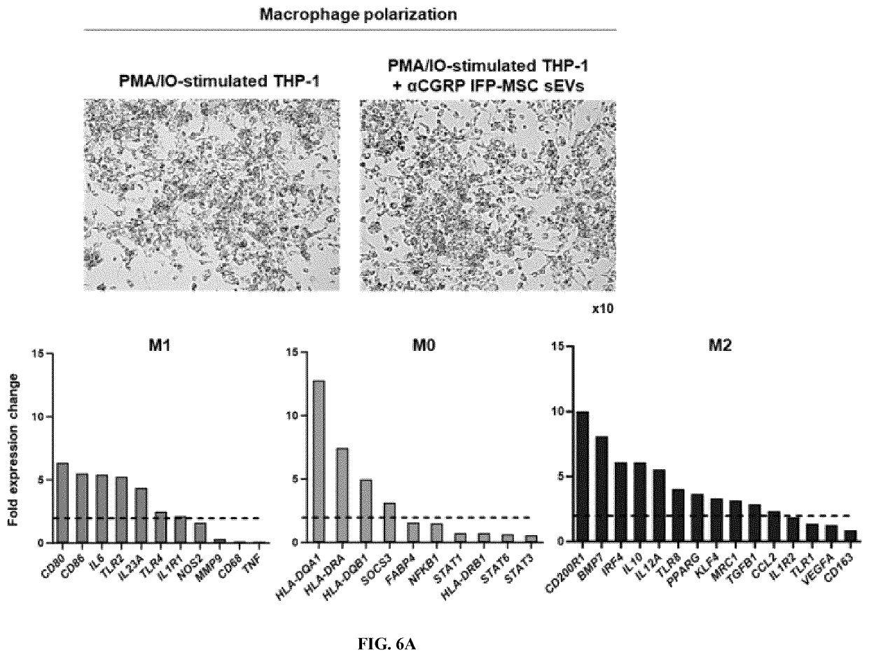

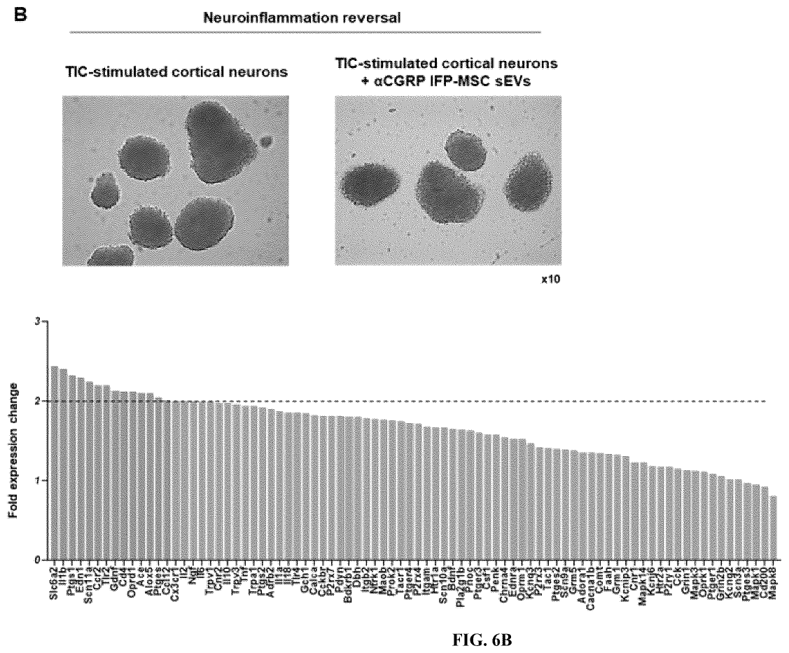

aCGRP IFP-MSC sEVs Effects on Macrophages PMA/IO-stimulated THP-1 showed similar morphology with and without aCGRP IFP-MSC sEVs treatment (FIG. 6A). However, their molecular profiling indicated a strong gene expression shift towards an M0/M2 macrophage polarization (FIG. 6A). Most importantly, the expression levels of CD200R1, BMP7, IRF4, IL10, IL12A, characteristic M2-polarization markers, were strongly induced when macrophages were exposed to aCGRP IFP-MSC sEVs. In parallel, 6 genes related to the M0 phenotype (HLA-DQA1, HLA-DRA, HLA-DQB1, SOCS3, FABP4, NFKB1) were highly upregulated upon aCGRP IFP-MSC sEVs exposure of PMA/IO-stimulated THP-1 cells. Stronger M2 macrophage polarization can be achieved by higher dose of SEVs (FIG. 6A).
aCGRP IFP-MSC sEVs Effects on Cortical Neurons

TIC-stimulated cortical neurons showed similar morphology with and without aCGRP IFP-MSC sEVs treatment (FIG. 6B). However, their molecular profiling indicated an overall reduced neuroinflammatory profile upon exposure to aCGRP IFP-MSC sEVs (FIG. 6B). From 84 genes, only 14 (SLC6A2, IL1B, PTGS1, EDN1, SCN11A, CCR2, TLR2, GDNF, CD4, OPRD1, ACE, ALOX5, PTGES, CCL12) showed increased expression (>2-fold) compared to TIC-stimulated cortical neurons alone. Interestingly, 4 major genes (MAPK8, CD200, MAPK1, PTGES3) involved in neuropathic pain were highly down-regulated (>2-fold) upon exposure to aCGRP IFP-MSC sEVs.

### Discussion

CGRP, a 37 amino-acid peptide, is well documented in playing a key role in the transmission of pain signals in both the periphery and the CNS. Accumulating evidence implicates a role for CGRP in the peripheral mechanisms of OA-mediated pain. Multiple knee joint structures are richly innervated by CGRP-expressing sensory neurons. A truncated form of the CGRP peptide, CGRP8-37, is a selective antagonist that binds to the CGRP receptor with approximately the same affinity as CGRP but does not result in receptor signal transduction.

IFP-MSC were successfully transduced which showed high expression of CD10 surface marker. CD10 is a neutral endopeptidase expressed in multiple cell types including MSC, with enzymatic activity neutralizing various signaling substrates including SP. SP is a neuropeptide associated with nociceptive pathways that is secreted by sensory nerve fibers in the synovium and IFP tissues. Upon its secretion it actively affects local inflammatory/immune and fibrotic responses by modulation of cell proliferation, activation and migration to sites of inflammation, and the expression of recruiting chemokines and adhesion molecules. IFP-MSC were previously reported to acquire a potent immunomodulatory phenotype and actively degrade SP via CD10 in vitro and in vivo. Therefore, high CD10 expression levels indicate that aCGRP IFP-MSC show increased functionality in SP degradation.

aCGRP IFP-MSC sEVs show high purity with <200 nm sizes and high expression levels of CD9 marker upon ultracentrifugation. Importantly, the successful transfer of AAV CGRP8-37 was shown within yielded sEVs. Predominantly, miRNAs present in sEVs are involved in the regulation of TGF-β/Wnt/FGFR pathways. TGF-β is a superfamily of growth factors that bind to activin receptor-like kinases (ALKs) and activate SMAD signaling. In OA, due to elevated TGF-β concentrations to which the chondrocytes are exposed to, the function of TGF-β changes from a factor that blocks chondrocyte hypertrophy (Smad2/3 signaling) to a factor that facilitates chondrocyte hypertrophy (Smad1/5/8 pathway). Recent evidence has suggested that WNT/β-catenin signaling may also play a role in the pathogenesis of OA. Canonical WNT pathway activation can play a prominent role in the proliferation of osteoblasts leading to osteophyte formation and chondrocytes hypertrophy. FGFR signaling plays an important role in the pathogenesis of OA as specifically FGFR1 is highly expressed in OA chondrocytes. In OA, FGF2 binding to FGFR1 leads to a cascade of events that are typically catabolic in articular chondrocytes, including the activation of mitogen-activated protein kinase (MAPK) pathways.

Importantly, miRNAs present in sEVs are also involved in the regulation of NGF and Toll-like receptor signaling. NGF has been discovered to have chemotropic effects on the CNS and have significant role in inflammatory conditions in terms of the sensation of pain and activation of the immune system. In OA, inflammatory stimuli enhance NGF release by chondrocytes whereas NGF is released in a dose and time-dependent relationship to IL-1β. Toll-like Receptors (TLR1-7, TLR9) have also been linked to the pathophysiology of OA as they are upregulated in the synovium of OA joints. Downstream to the upregulation of Toll-like receptors is the increased production of pro-inflammatory molecules such as NF-kB, TNF-α, and IL-1β. This, in turn, recruit's macrophages and immune cells to the site of the synovium. Toll-like receptor activity is also linked to the initiation of apoptosis; therefore, its high activity in the synovium might be linked to the apoptosis of chondrocytes, progressing the pathophysiology of OA.

In aCGRP IFP-MSC sEVs, 7 miRNAs cargos highly present were associated in previous studies with significant anti-inflammatory/immunomodulatory effects in vitro and in vivo. Specifically, hsa-miR-6089 targets the TLR4 mediated inflammatory response and inhibits the activation of macrophages through downregulation of IL-6, IL-29, and TNF-α cytokine production. Similarly, hsa-miR-21-5p and hsa-miR-23a-3p are also involved in the regulation of inflammatory cytokine production, such as IL-1β, IL-6 and TNF-α. Deficiency in hsa-miR-21-5p leads to increased recruitment of CD11b+ monocytes/macrophages and an increased inflammatory response. Both hsa-miR-99a-5p and miR-574-5p inhibit NLRP3 inflammasome by promoting macrophage autophagy through downregulation of mTOR signaling and by suppressing leukocytes infiltration and downregulating NF-κB. Also, miR-107 protects against inflammation and apoptosis of endothelial cells via KRT1-dependent Notch signaling pathway whereas hsa-let-7b-5p regulates neutrophil function and reduces neutrophilic inflammation by suppressing the canonical TLR4/NF-κB pathway. Lastly, hsa-miR-1290 is directly related to the differentiation status of MSC undergoing chondrogenic differentiation indicating its putative chondroprotective and anabolic effects for cartilage regeneration in vivo.

Multiple immunomodulatory proteins (TIMP-2, IL-8, MCP-1, IL-6, ICAM-1, sTNF-RI, MIP-1β, IL-10, and IP-10) were detected to be secreted as a cargo of aCGRP IFP-MSC SEVs. The highest present protein, TIMP-2, attenuates the development of inflammation and inflammatory mediated pain via MMP-dependent and receptor-mediated cell signaling mechanisms. Specifically, NF-κB, Nrf2, and CREB pathways are largely involved in TIMP-2-mediated anti-inflammatory effects. When TIMP-2 is knocked out in OA models, there is increased angiogenesis and increased degeneration of the articular cartilage. Also, IL-6 and IL-8 cytokines have been implicated in both pro- and anti-inflammatory actions with previous studies indicating that the IL-6/IL-8 ratio plays a crucial role in the specific polarization of the cellular microenvironment. MCP-1 and ICAM-1 are associated with monocyte recruitment and adhesion in inflamed tissues after pro-inflammatory cytokine activation. Importantly, MCP-1 and CCR2 chemokine receptors determine the extent of M2 macrophage polarization by enhancing the production of the anti-inflammatory IL-10 cytokine. Also, studies have illustrated that IP-10 secretion directly correlates with decreased T cell proliferation. Importantly, IL-10 inhibits MHC class II and costimulatory molecule B7-1/B7-2 expression on monocytes and macrophages and limits the production of pro-inflammatory cytokines.

Key reparative proteins (HGF, VEGF, EGFR, IGFBP-1, BFGF, and IGFBP-6) showed also presence in aCGRP IFP-MSC sEVs. HGF is a multifunctional growth factor that, like its specific receptor c-Met, is widely expressed in joint tissues. Studies showed that HGF promotes cell proliferation and survival in chondrocytes while inhibiting ECM degradation of articular tissue, both of which are key regulators of degenerative changes in OA. EGFR is critical for the maintenance of homeostasis of the superficial layer of articular cartilage. Animal models lacking EGFR have fewer superficial chondrocytes, less secretion of boundary lubrication, weak mechanical strength at the cartilage surface, and continued OA progression. Interestingly, VEGF secreted by MSC induces endothelial progenitor cell differentiation towards endothelial cells via paracrine actions. IGF-1 promotes cartilage anabolism by inducing proteoglycan synthesis in chondrocytes via PI3K pathway and by stimulation of chondrocyte proliferation via the PI3K and MEK/ERK pathways. The two highly present IGF-1 binding proteins, IGFBP-1 and IGFBP-6, extend the plasma half-life of IGF-1 and therefore enhancing/prolonging its anabolic effects in vivo. The role of BFGF for articular cartilage homeostasis is controversial. However, recent studies have shown that BFGF can induce the expression of TIMP1 gene (which encodes an inhibitor of MMPs) in cartilage and can suppress IL-1-induced expression and activity of ADAMTS4 and ADAMTS5, enzymes that degrade aggrecan in human articular cartilage.

In the present disclosure, it is shown that aCGRP IFP-MSC sEVs can effectively polarize M1 macrophages to M2 anti-inflammatory phenotype. Most importantly, the expression levels of CD200R1, BMP7, IRF4, IL10, IL12A, characteristic M2-polarization markers, were strongly induced upon aCGRP IFP-MSC sEVs exposure. Both CD200R1 and BMP7 induce M2 macrophage polarization, whereas BMP7 significantly increase the expression of anti-inflammatory markers, arginase-1 and IL-10. IRF4 has been shown to regulate the expression of glycolytic genes, suggesting a conserved link between mTOR signaling, IRF4 and metabolic reprogramming in immune cells. Also, studies showed that IL-10-induced M2 macrophage phenotype implicate intracellular protein BCL3. Finally, studies showed that IL12A deletion significantly exacerbates macrophage accumulation, polarize macrophages to M1 phenotype and increase the expression levels of macrophage-secreted proinflammatory cytokines.

The truncated CGRP peptide, CGRP8-37, can act as a CGRP antagonist. In the present disclosure, inflamed cortical neurons exposed to aCGRP IFP-MSC sEVs treatment showed an overall reduced neuroinflammatory profile. Interestingly, 4 major genes (MAPK8, CD200, MAPK1, PTGES3) involved in neuropathic pain were highly down-regulated (>2-fold) upon exposure to aCGRP IFP-MSC sEVs. Mitogen-activated protein kinases (MAPKs) are a family of serine/threonine protein kinases that play essential roles in inflammation and tissue remodeling and are involved in the modulation of nociceptive signaling and the peripheral/central sensitization produced by intense noxious stimuli. Previous studies have demonstrated that the inhibition of MAPKs produces anti-inflammatory effects in various inflammatory diseases. MAPKs activation contributes to pain sensitization via both neuronal and glial mechanisms that may be mediated by their different isoforms, such as MAPK1 and MAPK8. These two isoforms were downregulated following sEVs treatment. Notably, studies showed that MAPK8 activation in astrocytes contributes to the maintenance of chronic pain. In parallel, MAPK1 has been recognized as key molecule of pain signaling that can induce alone inflammatory pain but cooperates with MAPK3 in sensory neuron survival.

In summary, an alteration at the neuroinflammatory molecular profile of cortical neurons was shown upon exposure to aCGRP IFP-MSC sEVs. Therefore, aCGRP IFP-MSC sEVs strongly indicate their potent immunomodulatory functionality.

### Conclusions

Cell sorting of AAV transduced IFP-MSC yielded purified cultures of CD10-bound IFP-MSC expressing GFP tagged gene encoding for a competitive antagonist of CGRP. These cells yielded aCGRP IFP-MSC sEVs with distinct miRNA and protein signatures as cargos. In silico analysis demonstrated that detected miRNAs and proteins regulate multiple pathways and biological processes involved in the control of pain, inflammation, and cartilage homeostasis.

Additionally, aCGRP IFP-MSC sEVs effectively polarize M1 macrophages to the M2 phenotype and alter the neuroinflammatory profile of stimulated cortical neurons. The sEVs as described herein can target CGRP signaling involved in inflammation and pain in vivo.

### Example 2. Behavioral Pain Analyses Using Vectors Encoding CGRP8-37

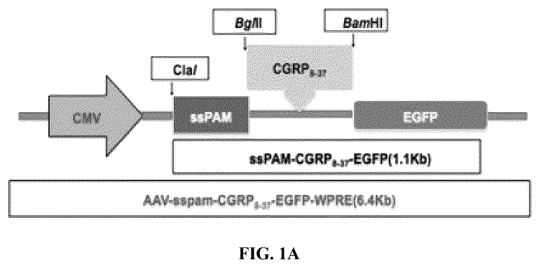

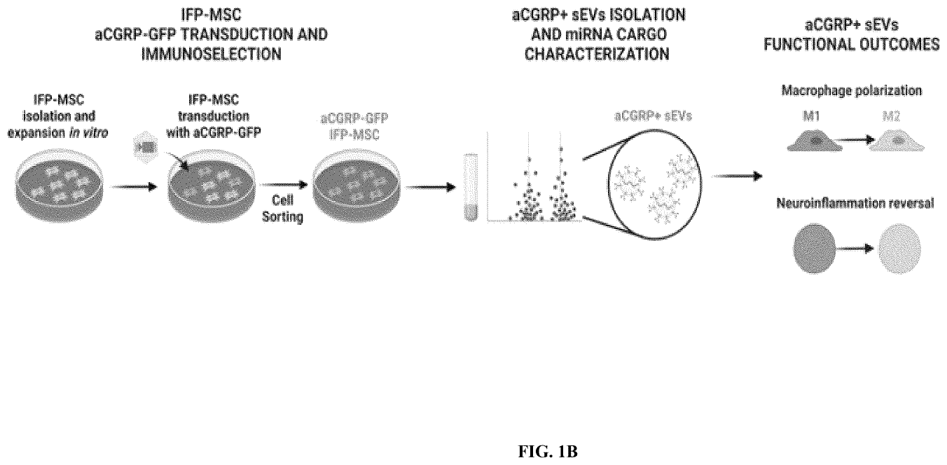

As described herein, a truncated CGRP peptide, CGRP8-37, acts as a CGRP antagonist and has been shown to reverse symptoms of neuropathic, inflammatory, and migraine pain. In one aspect, the present disclosure provides a DNA fragment encoding CGRP8-37 cDNA which was generated, sequenced, and evaluated as a chronic pain gene therapy (see, e.g., FIG. 1A of Example 1).

To accomplish this, oligonucleotides specific to CGRP8-37 were used in a standard PCR to amplify the region from a human CGRP cDNA containing plasmid. The PCR product (~156 bp) flanked by BglII and BamHI restriction sites (FIG. 1A) was subcloned into pGemT EZ vector (Promega), amplified and sequenced. Vectors with proper sequence were then digested and ssPAM signal sequence was inserted upstream, allowing CGRP8-37 to be secreted (FIG. 1A). The ssPAM-CGRP8-37 was excised from pGemT EZ vector by ClaI/BamHI digestion and subcloned into a pAAV2-EGFP-WPRE plasmid. The addition of EGFP was to allow visualization of transduced cells using EGFP reporter. AAV recombinant vector was amplified by Stable3 bacterial cloning, digested and ssPAM-CGRP8-37 insert extracted and sequenced. The sequence of the final vector, SEQ ID NO: 27, was checked for the proper orientation and alignment of the inserts. Similarly, lentiviral vectors were also used as an alternative recombinant viral vector delivery system, resulting in the full engineered Lentiviral vector sequence of SEQ ID NO: 28.

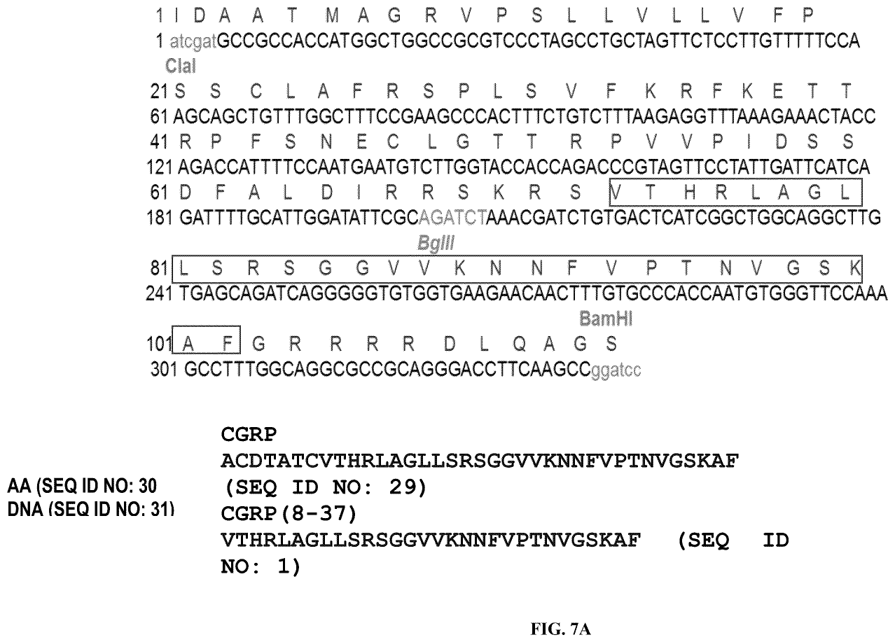

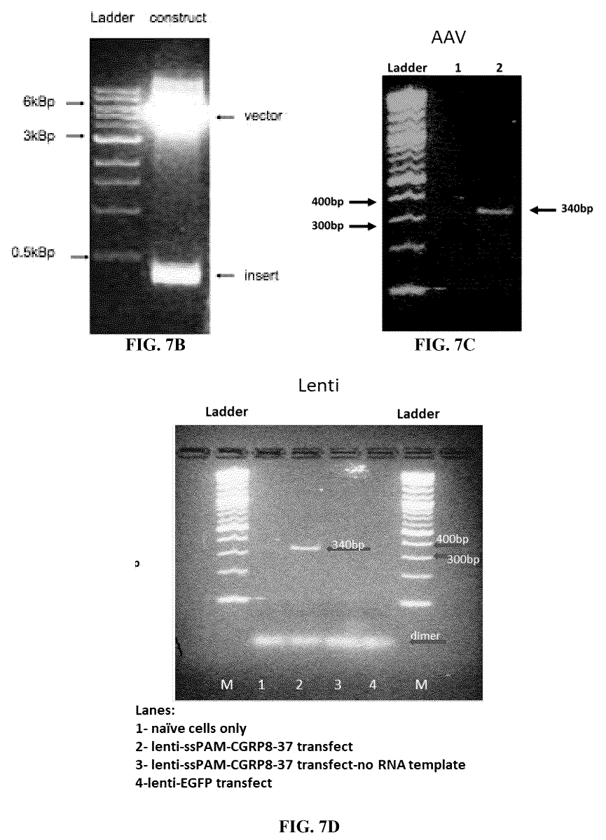

The AAV recombinant plasmid was tested by transfecting HEK 293T cells to evaluate the expression of the CGRP8-37 insert in vitro. cDNA gel electrophoresis of ClaI-BamHI digested AAV-sspam-CGRP8-37-EGFP construct shows fragments of AAV vector (5.9 Kb) and sspam-CGRP8-37 insert (0.34 kb) (FIG. 7B), confirming correct location of the insert and stable design of the plasmid. Gel electrophoresis of RT-PCR amplified with template for CGRP (8-37) isolated from cell culture supernatant was performed to detect the presence of recombinant CGRP (8-37) in the supernatant. HEK293T cells were transfected with AAV vector (lane 1) and AAV-sspam-CGRP8-37-EGFP (lane 2; SEQ ID NO: 27). The recombinant insert, sspam-CGRP8-37 (340 bp), was only detected in cells transfected by the recombinant AAV vector (FIG. 7C).

The lentiviral recombinant plasmid was also tested by transfecting rat embryonic neuronal progenitor cells with Lenti-sspam-CGRP (8-37). Gel electrophoresis of RT-PCR isolated from cell cultures and amplified with template for CGRP (8-37) to detect its presence in the supernatant. The recombinant insert, sspam-CGRP8-37 (340 bp), in the supernatant of transfected cells (FIG. 7D).

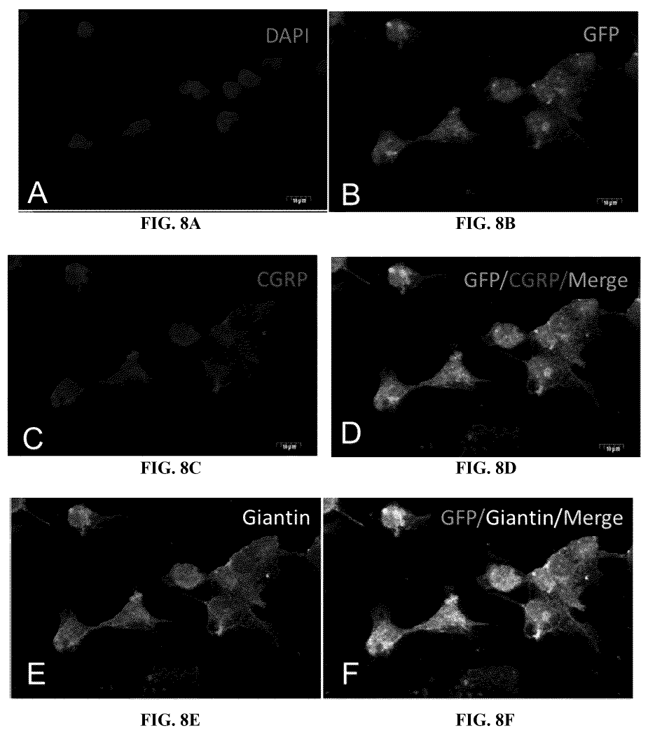

Immunofluorescence (IF) staining on HEK293T cells transfected with AAV-sspam-CGRP8-37-EGFP construct showed co-localization of GFP (recombinant CGRP (8-37)) (FIG. 8B) with all CGRP (FIG. 8C) in the region around the nucleus as identified by DAPI staining (FIG. 8A), suggesting that recombinant CGRP (8-37) peptide was successfully expressed (FIG. 8D). Co-staining of GFP (CGRP (8-37)) with the Goli marker Giantin (FIG. 8E) indicated that the recombinant CGRP (8-37) peptide is being synthesized in the secretory pathway.

Confocal imaging of transfected HEK293T cells stained with anti-CGRP antibody and DAPI showed the successful synthesis of CGRP (8-37), and divergent localization of CGRP and recombinant CGRP (8-37) peptide in the peripheral cytoplasm. Importantly, the CGRP8-37 and EGFP have an intervening dipeptide that facilitates the division of the single fusion protein into its separate components.

Next, a Fluorescence-linked immunosorbent assay (FLISA) was conducted on dilutions of supernatant of HEK293T cells transfected with recombinant and nonrecombinant AAV vector with CGRP (8-37) fragment. Dose dependent staining of CGRP was observed with increasingly concentrated supernatant sample harvested from cells transfected with the AAV-sspam-CGRP8-37-EGFP construct, whereas non-transfected (naïve) or cells transfected with AAV-GFP had no marked staining (FIG. 10). These results independently confirm expression of recombinant CGRP (8-37) peptide.

Following confirmation of successful recombinant CGRP (8-37) peptide, several evaluations of behavioral pain in animal models were conducted.

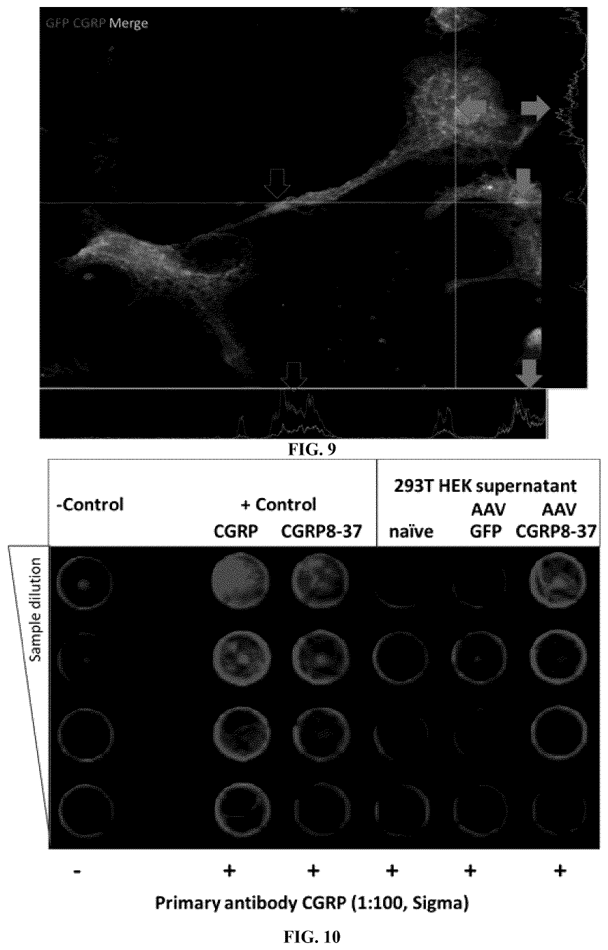

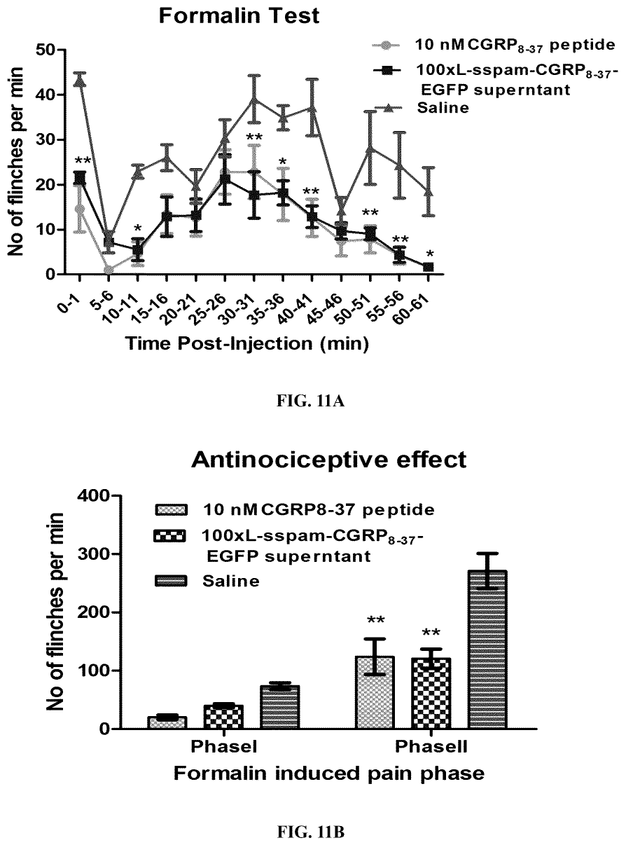

Supernatant was collected and used for intrathecal injection in rats with peripheral nerve injury to evaluate analgesic effects of the recombinant CGRP (8-37) produced by transfected cells, and compared with intrathecal injection of the synthetic CGRP (8-37) peptide. Acute inflammation was induced by 5% formalin injected into rat hind paw to model neurogenic inflammation. Pain-related behavior (flinching, licking of the paw) was observed and recorded for 60 min post injection. Supernatant/control CGRP8-37 peptide/saline was injected intrathecally 15 min before the formalin injection. Formalin-induced behavior was fully developed in control (saline-injected) animals, whereas the behavior was significantly reduced in animals injected with either control CGRP (8-37) peptide or cell supernatant from cells transfected with AAV-ssPAM-CGRP (8-37)-EGFP (FIG. 11A), indicating the presence of active, recombinant CGRP (8-37) peptide in the supernatant. Further analysis of Phase I (0-5 min) and Phase 2 (20-60 min) shows a significant reduction of pain-related behavior in animals injected with control peptide and AAV-ssPAM-CGRP (8-37)-EGFP supernatant.

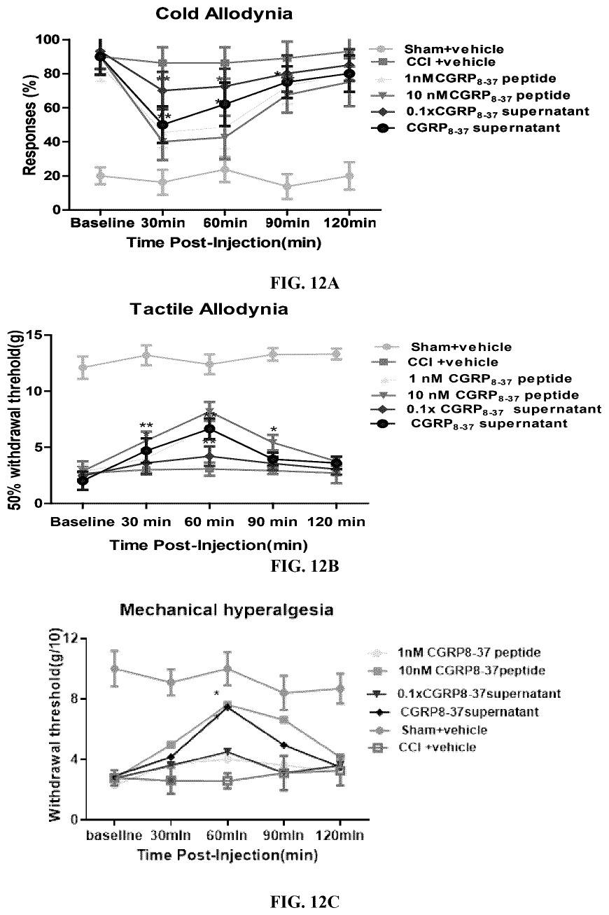

Separately, intrathecal injections of supernatant from AAV-ssPAM-CGRP (8-37)-EGFP-transduced HEK cells reduce neuropathic pain metrics induced by chronic constriction injury (CCI) of the sciatic nerve, including cold allodynia (FIG. 12A), tactile allodynia (FIG. 12B), and mechanical hyperalgesia (FIG. 12C). The reduction in pain metrics in CCI rats treated with CGRP8-37 supernatant were comparable to intrathecal injection of authentic CGRP peptide, whereas no alleviation of pain behavior was seen in untreated CCI rats.

Recombinant CGRP (8-37) supernatant has been tested via intrathecal injection in a rat model of neurogenic inflammation using the formalin test. In all cases, excellent analgesic effects were obtained; thus the plasmid was then used to engineer recombinant lenti-sspam-CGRP8-37-EGFP- or AAV2/8 viral particles.

In some embodiments, the compositions as described herein are administered by the intrathecal route. In some embodiments, the compositions as described herein are administered by the intra-DRG route, wherein intra-DRG administrations are potent for regional pain via CGRP antagonism in both the spinal and peripheral processes of the pain nociceptors.

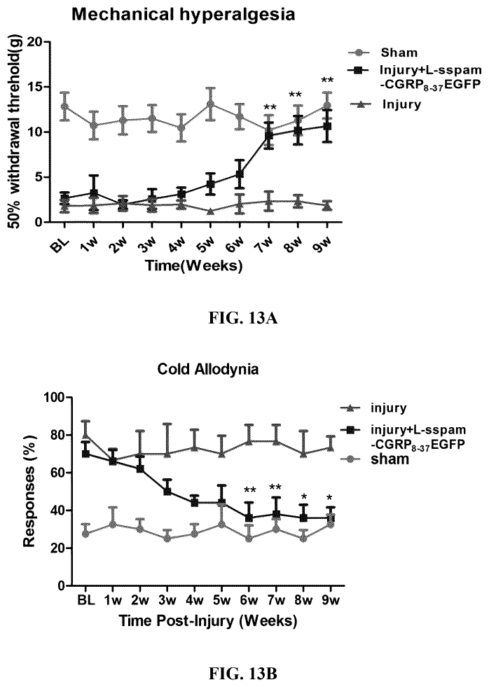

Next, the Lenti-sspam-CGRP8-37-EGFP was analyzed in a spinal cord injury (SCI) model. SCI rats were intraspinally injected with the either L-sspam-CGRP8-37EGFP or control Lenti-EGFP into L3-L4 lumbar dorsal horn, while sham animals were not injected. Substantial attenuation of mechanical hyperalgesia (FIG. 13A) and cold allodynia (FIG. 13B) was observed by 6-7 weeks post-injection. Gradual improvement towards pre-injury levels were achieved by 9 weeks post-SCI.

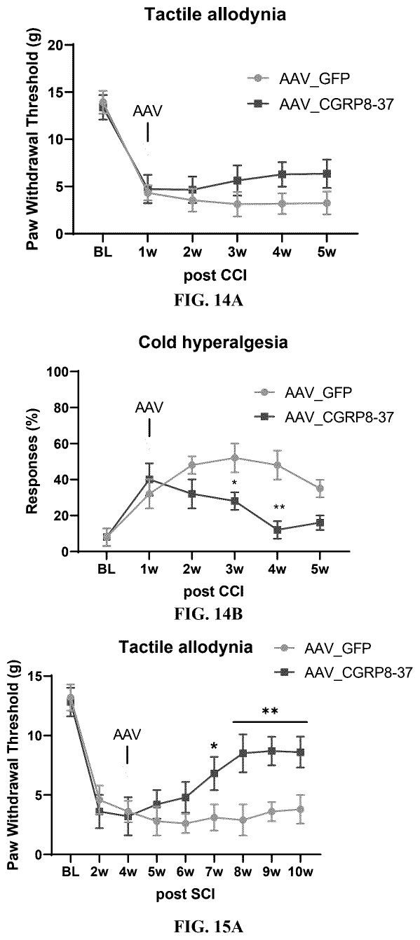

Next, chronic constriction injury (CCI) was induced in animals to mimic pain after peripheral neuropathy. AAV construct with either CGRP8-37 or control GFP gene was injected intraspinally into lumbar L3-L4 spinal cord at 1 week post CCI injury. The responses of animals to tactile and cold stimuli were evaluated. Attenuated tactile allodynia (FIG. 14A) and cold hyperalgesia (FIG. 14B) were observed in animals treated with the AAV-sspam-CGRP8-37-EGFP construct.

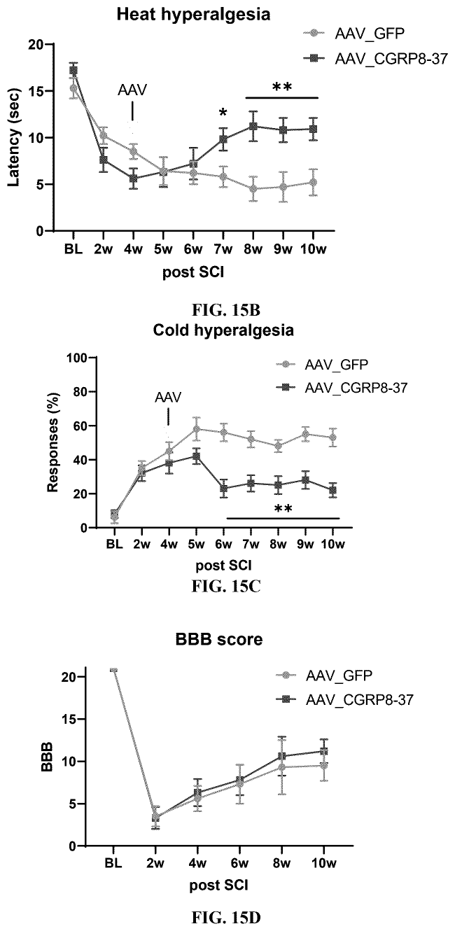

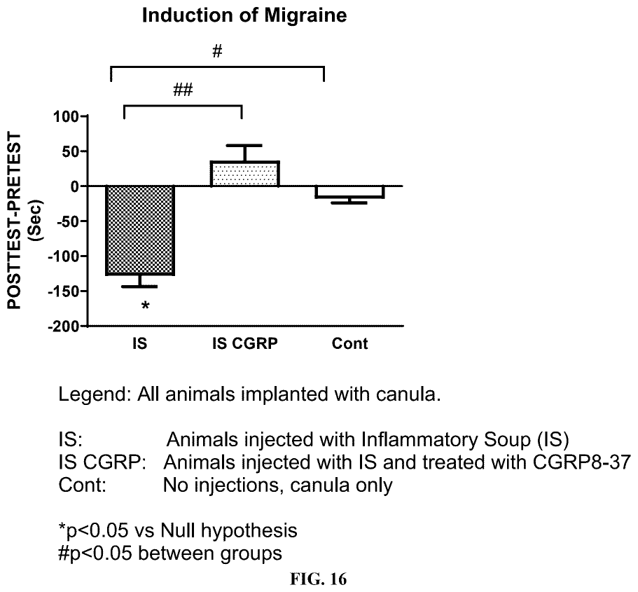

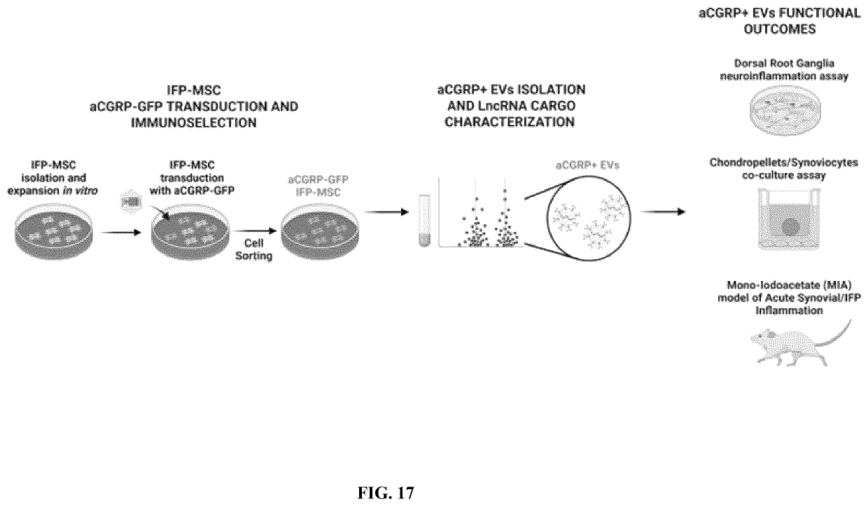

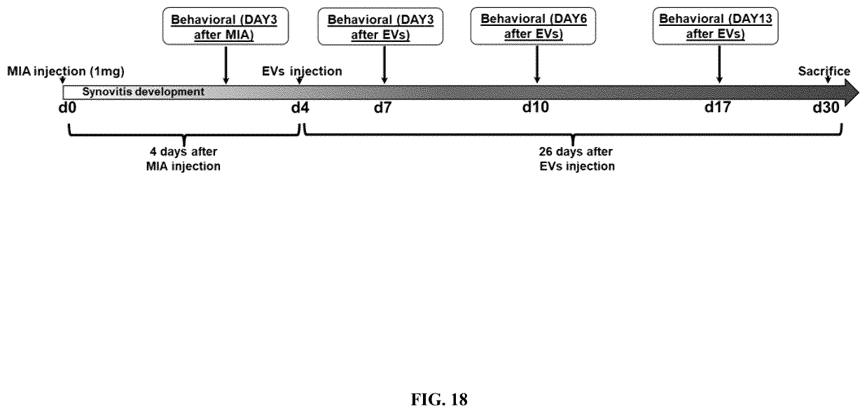

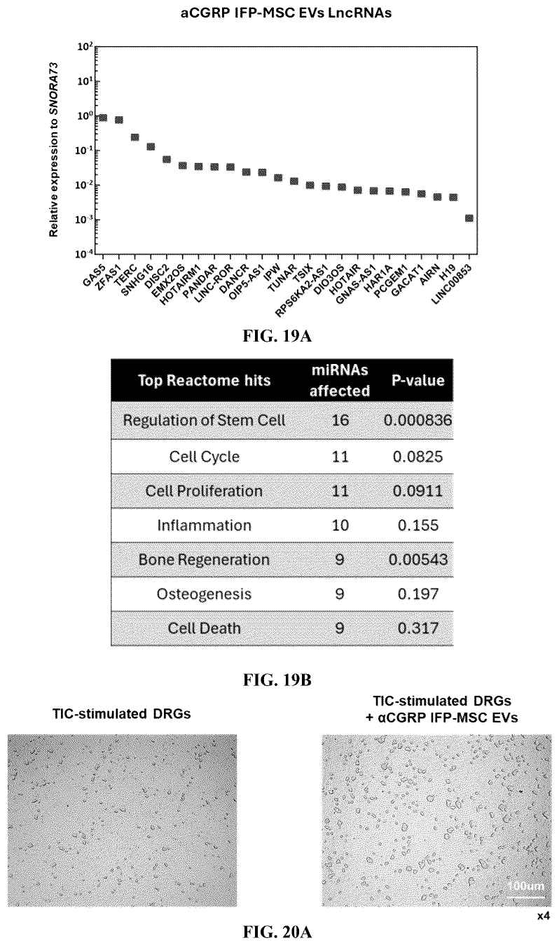

Next, spinal cord clip injury was induced in animals to mimic below level SCI pain. AAV construct with either CGRP8-37 or control GFP gene was injected intraspinally into lumbar L3-L4 spinal cord at 4 weeks post-injury. The responses of animals to tactile, cold, and heat stimuli were evaluated, along with locomotor scores (FIG. 15D). Significantly attenuated tactile allodynia (FIG. 15A), heat hyperalgesia (FIG. 15B), and cold hyperalgesia (FIG. 15C) were observed in animals administered the AAV-sspam-CGRP8-37-EGFP construct compared to AAV-GFP control, starting generally around 7 weeks (approximately 3-weeks post-AAV injection). The safety of the treatment was confirmed by evaluation of locomotor scores, in which both AAV-sspam-CGRP8-37-EGFP construct and AAV-GFP control groups were similar.

For both SCI and CCI models, the recombinant viral vectors were delivered directly to the spinal dorsal horn pain processing regions via the intra-spinal route. Chronic neuropathic pain symptomology (tactile allodynia, cold allodynia) was significantly attenuated and this reduced pain was maintained for the duration of the study. No adverse effects on locomotor coordination were found.

A similar intraganglionic approach was tested in pilot studies using the migraine headache rat model. Animals were cannulated and inflammatory soup (IS) injected over the brain dura to induce migraine-like behavior. Administration of AAV-CGRP8-37 via injection in the trigeminal (gasserian) ganglion in a conditioned place preference (light aversion) test 25 days post injection of IS. In humans, the intragasserian approach can be developed as a minimally invasive procedure, since local anesthetics, steroids, and others are routinely injected percutaneously into the trigeminal ganglia for diagnostic and therapeutic procedures.

Conditioned place preference was evaluated in animals; time spent in the light side of the testing cage over the darker side was recorded before and after the treatment. Animals with IS injection only developed a strong preference towards the darker side. In contrast, animals with IS injection treated with AAV-CGRP8-37 construct did not develop such behavior and their values were similar to control animals. The values in both treated and control animals were significantly different from untreated animals with IS injection.

### Conclusions

In summary, the recombinant AAV-CGRP8-37 or lenti-CGRP8-37 was tested in several animal models of chronic pain, including spinal cord injury pain (SCI), peripheral neuropathic pain using a chronic constriction sciatic nerve injury (CCI), and migraine using a subdural inflammatory soup (IS) model. In some embodiments, the compositions and methods as described herein are used for the treatment of knee osteoarthritis (OA) comprising direct intra-articular (knee joint) injection administrations.

In all cases, continued expression of the transgene, including co-localization of CGRP8-37 and EGFP reporter in ganglionic neurons and nerve fibers, was observed.

Accumulating evidence implicates a role for CGRP in both the central and peripheral mechanisms of OA-related pain. For example, CGRP nerve fiber density is increased in a time-dependent fashion following increased nerve growth factor (NGF) levels and is tissue specific to the OA joint itself. Higher levels of CGRP can then act on a wide variety of cell types in the joint. It can inhibit osteoclastogenesis, thus contributing to bone sclerosis and disease progression. CGRP released by nerve fibers can also act on the fibers themselves, enhancing pain sensitization. CGRP release is calcium dependent, so overexpression of calcium channel modulators can reduce the pain threshold in rat MIA models of arthritis via downregulation of CGRP pathways.

### Example 3: Functional Assessment of Genetically Modified Infrapatellar Fat Pad Mesenchymal Stem/Stromal Cell Derived Extracellular Vesicles (EVs): Potential Implications for Inflammation/Pain Reversal in Osteoarthritis

Osteoarthritis (OA) is a debilitating joint disease affecting over 500 million people globally, characterized by cartilage degradation, chronic pain, and failed tissue repair. Neurogenic inflammation, driven by neuropeptides including Substance P (SP) and calcitonin gene-related peptide (CGRP), plays a key role in the pathogenesis of OA. The present application investigates the therapeutic potential of extracellular vesicles (EVs) derived from infrapatellar fat pad mesenchymal stem/stromal cells (IFP-MSC) transduced with CGRP antagonist CGRP8-37 (aCGRP IFP-MSC EVs). These EVs are enriched in anti-inflammatory miRNAs and proteins, and express neprilysin (CD10), enabling SP degradation. Herein, several LncRNAs were identified, which have been known to interact with miRNAs that affect the knee joint homeostasis. Specifically, 11 LncRNAs (ZFAS1, EMX2OS, HOTAIRM1, RPS6KA2-AS1, DANCR, LINC-ROR, GACAT1, GNAS-AS1, HAR1A, OIP5-AS1, TERC) interact with miRNAs that promote cell proliferation, prevent apoptosis, and preserve homeostasis. In vitro, aCGRP EVs downregulated pro-inflammatory markers (TNF, TLR4, MAPK8) in dorsal root ganglia and promoted chondrocyte gene expression consistent with anabolism and matrix remodeling. In vivo, intra-articular EV delivery attenuated pain behaviors, preserved cartilage structure, restored PRG4+ stem/progenitor cell localization, and trended toward reduced SP levels. Histological analysis confirmed improved collagen organization and reduced matrix degradation. These findings indicate that aCGRP IFP-MSC EVs exert multimodal effects on neuroinflammation, cartilage regeneration, and joint homeostasis. This cell-free, gene-enhanced EV therapy offers a disease-modifying strategy for treatment of OA, with potential to address both structural changes and chronic pain associated with this disease.

Knee osteoarthritis (OA) affects over 500 million people worldwide, contributing to disability and economic burden. According to WHO, about 73% of people living with OA are older than 55 years, and 60% are female. The pathogenesis of OA includes nociceptive signaling, neurogenic inflammation, and immune dysregulation. The synovium and infrapatellar fat pad (IFP) become sites of immune infiltration, releasing pro-inflammatory and cartilage-degrading molecules, along with Substance P (SP) and calcitonin gene related peptide (CGRP)-two neuropeptides central to pain sensitization and joint degeneration.

SP plays a critical role in nociception and promotes local inflammation by increasing vascular permeability and driving macrophage polarization towards the M1 pro-inflammatory phenotype, which can affect synovial inflammation and cartilage degradation-key processes in the progression of OA. Similarly, CGRP contributes to both central and peripheral pain mechanisms, with increased CGRP immunoreactive nerve fiber density observed in OA synovial tissue. In OA, CGRP levels rise in response to joint injury, amplifying pain signaling and contributing to aberrant bone remodeling by inhibiting osteoclastogenesis, which may accelerate sclerosis and disease progression.

CGRP₈₋₃₇, a CGRP antagonist (aCGRP), has exhibited analgesic effects across multiple pain models. Gene therapy approaches using viral vectors, including adenoassociated virus (AAV), offer a promising strategy for long-term CGRP₈₋₃₇ expression, with an excellent safety profile in over 130 clinical trials. Meanwhile, SP activity is regulated by the membrane-bound neutral endopeptidase CD10 (neprilysin), expressed by mesenchymal stem/stromal cells (MSC). Previous work demonstrated that IFP-MSC acquired an immunomodulatory phenotype and degraded SP via CD10 in vitro and in vivo by cleaving SP at the amino side of its hydrophobic residues. Notably, CD10High extracellular vesicles (EVs) exhibit miRNA-driven immunomodulatory and anabolic properties that support joint homeostasis. In parallel, IFP-MSC has successfully been transduced with the CGRP antagonist, CGRP₈₋₃₇, and it was confirmed that isolated EVs contain the CGRP antagonist (aCGRP IFP-MSC EVs). Importantly, aCGRP IFP-MSC EVs were enriched in miRNAs and proteins known to exert potent anti-inflammatory, immunomodulatory, and cartilage homeostasis effects. These EVs also were able to polarize macrophages away from the proinflammatory M1 phenotype towards the anti-inflammatory M2 phenotype.

It was hypothesized that aCGRP IFP-MSC EVs attenuate inflammation and pain signaling in vitro, and mitigate OA progression in an in vivo rat model of acute synovial/IFP inflammation. Herein, the data indicate that aCGRP IFP-MSC EVs attenuate neuroinflammation of dorsal root ganglia (DRGs), and alter gene expression of chondrocytes exposed to a pro-inflammatory environment in favor of cartilage anabolism. Also, aCGRP IFP-MSC EVs intra-articular infusion results in reduced pain signaling and preserved cartilage structure.

### Materials and Methods

### Isolation, Culture and Expansion of IFP-MSC

All experiments using human cells were performed in accordance with relevant guidelines and regulations. Human IFP-MSC were isolated from IFP tissue obtained from deidentified, non-arthritic patients (n=5 donors; two males 26 and 48 years old, and three females 22, 42, 44 years old) undergoing elective knee arthroscopy at the Lennar Foundation Medical Center at the University of Miami. All procedures were carried out following approval by the University of Miami IRB not as human research (based on the nature of the samples as discarded tissue). The isolation and culture expansion of IFP-MSC are described.

### Generation of AAV Vector Containing GFP-Labeled CGRP Antagonist Gene

CGRP₈₋₃₇ is a truncated polypeptide of CGRP that serves as a competitive antagonist of CGRP (aCGRP). The generation of AAV vector containing GFP-labeled CGRP antagonist gene is described.

IFP-MSC Transduction and Cell Sorting for GFP Positive aCGRP IFP-MSC

AAV-aCGRP-GFP was used to transduce 20×10⁶ P1 IFP-MSC (n=5 donors). Transduced cultures were maintained at 37° C., 5% (v/v) CO2 for 7 days and GFP fluorescence was visualized using ×10 objective Leica DMi8 microscope with Leica X software (Leica). On day 7, the transduced IFP-MSC were sorted based on GFP expression to yield the aCGRP IFP-MSC subpopulation using MoFlo Astrios EQ cell sorter (Beckman Coulter, Brea, CA, USA). After sorting, aCGRP IFP-MSC were seeded on 0.1% gelatin plates (MilliporeSigma, St Louis, MO, USA) and were cultured at 37° C., 5% (v/v) CO2 until 80% confluency.

Isolating EVs from aCGRP IFP-MSC

EVs were isolated from aCGRP IFP-MSC conditioned media by a stepwise ultracentrifugation method. The isolation and biophysical and biochemical characterization using flow cytometry and nanoparticle tracking analysis of EVs from aCGRP IFP-MSC is described. IFP-MSC EVs yield was 3-4×10⁹/ml as detected by nanoparticle tracking analysis for all IFP-MSC donors.

### Long Non-Coding RNAs (LncRNAs) Assay

RNA was isolated from aCGRP IFP-MSC EVs using the Total Exosome RNA and Protein Isolation Kit (Thermo Fisher Scientific) according to manufacturer's instructions. Then cDNA was synthesized with All-in-One miRNA First-Strand cDNA Synthesis Kit (GeneCopoeia, Rockville, MD, USA).

A pre-designed RT2 lncRNA PCR Array Human IncFinder (QIAGEN) was performed using 500 ng cDNA per IFP-MSC sample per manufacturer's instructions and processed using StepOne Real-time thermocycler (Applied Biosystems, LLC, Waltham, MA, USA). Data analysis was performed using qPCR results with GeneCopoeia's online Data Analysis System (accessed on 2 Jun. 2025)). Mean values were normalized to small nucleolar RNA SNORA73 and expression levels were calculated using the 2^{-ΔCt} method. Putative interactomes were generated using a miRNet centric network visual analytics platform (mirnet.ca/(accessed on 2 Jun. 2025). The lncRNA target gene data were collected from well-annotated database. Values (with 34 cycles cut-off point) were represented in a topology lncRNA-gene interactome network using force atlas layout and hypergeometric test algorithm.

### Dorsal Root Ganglia (DRGs) Neuroinflammation Assay

Rats were perfused with 4% paraformaldehyde in 0.1 M PBS, and DRGs were removed and isolated. 1.0×10⁵ TIC inflammatory/fibrotic cocktail (15 ng/ml TNFα, 10 ng/ml IFNγ, 10 ng/ml CTGF)-stimulated DRGs were mixed with aCGRP IFP-MSC EVs (at a concentration corresponding to EVs secreted from 1×10⁶ IFP-MSC or 3-4×10⁹ EVs/ml, n=2 donors) per well of 12-well plate and cultured for 3 days.

RNA extraction and cDNA synthesis from TIC-stimulated DRGs were performed using the RNeasy Mini Kit (Qiagen) and SuperScript™ VILO™ cDNA synthesis kit (Invitrogen), respectively. A predesigned 84 genes rat neuropathic and inflammatory qPCR array (RT2 Profiler neuropathic & inflammatory array, Qiagen) was performed using a StepOne Real-time thermocycler (Applied Biosystems, LLC). Mean values were normalized to ACTB. Expression levels were calculated as the relative fold change of the TIC-stimulated DRGs treated with aCGRP IFP-MSC EVs to TIC-stimulated DRGs alone (reference sample, 2^{-ΔCt}=X sample/X reference sample).

### Chondropellets/Synoviocytes Co-Culture Assay

Chondrogenesis was performed by inducing with a serum-free chondrogenic differentiation medium (MesenCult™-ACF Chondrogenic Differentiation Kit, STEMCELL Technologies, Vancouver, Canada) 0.25×10⁶ IFP-MSC per pellet for 15 days. After differentiation, the resulting chondropellets were collected and placed into a transwell co-culture system alongside synoviocytes, either with or without the addition of aCGRP IFP MSC EVs (at a concentration corresponding to EVs secreted from 1×10⁶ IFP-MSC or 3-4×10⁹ EVs/ml, n=2 donors). The co-cultures were maintained in synoviocyte growth medium supplemented with a pro-inflammatory and fibrotic cytokine cocktail consisting of TNFα (15 ng/ml), IFNγ (10 ng/ml), and CTGF (10 ng/mL) for a duration of 72 hours.

After three days, the chondropellets were harvested for histological evaluation and gene expression analysis. For histology, the pellets were cryosectioned at 6 μm thickness and stained using hematoxylin and eosin (H&E) for general tissue structure and 1% toluidine blue for assessing chondrogenic matrix production. For molecular profiling, RNA was extracted using the RNeasy Mini Kit (Qiagen, Frederick, MD, USA), followed by cDNA synthesis using the SuperScript™ VILO™ kit (Invitrogen). A predesigned 88 genes human osteoarthritis and cartilage repair qPCR array (GeneQuery™ Human Osteoarthritis and Cartilage Repair qPCR Array Kit, ScienCell) was performed using a StepOne Real-time thermocycler (Applied Biosystems). Mean values were normalized to ACTB. Results were presented as stacked bar graphs, showing fold changes in expression in aCGRP IFP MSC EVs-treated pellets relative to untreated controls.

### Mono-Iodoacetate (MIA) Model of Acute Synovial/IFP Inflammation

All animal procedures were approved by the Institutional Animal Care and Use Committee (IACUC) at the University of Miami and followed ARRIVE guidelines. A GraphPad Random Numbers calculator has been used to randomly assign animals for surgeries and for the subsequent cell treatments within the groups. The order of surgeries was randomized until the appropriate number of animals in each group was reached. Behavioral testing was conducted as stated by an experimenter blinded to surgical/treatment group. Sigma Stat software was used to calculate the sample size for each proposed aim. Input data were used based on preliminary data with OA and MIA pain models and experiences with behavioral tests in other pain-related animal models.

24 male Sprague Dawley rats were housed individually in a controlled environment (12/12 hr light/dark cycle, ad libitum food/water) for one week before being separated into three experimental groups, 8 receiving only MIA injection (negative control), 8 receiving CD10High EVs after MIA injection (positive control), and 8 receiving aCGRP EVs after the MIA injection. Synovial and IFP inflammation was induced via intra-articular injection of 1 mg monoiodoacetate (MIA) in 50 μL saline into the right knee. Under isoflurane anesthesia, injections were administered at 90° knee flexion using the patellar ligament and joint line as anatomical landmarks. Four days later, a single 100 μL intra-articular injection of aCGRP EVs at a concentration corresponding to EVs secreted from 1×10⁶ IFP-MSC or 3-4×10⁹ EVs/ml.

### Behavioral Assessments

Animal well-being was monitored daily post-injections. All behavioral tests were conducted by blinded evaluators. To assess tactile and cold allodynia, rats were placed on a metal mesh platform enclosed in a plastic dome (13× 17×28 cm) and allowed to acclimate for at least 20 minutes before testing. Behavioral responses were evaluated at baseline, 3 days after MIA injection and 3, 6, and 13 days post-EV treatment, then scarified at 30 days after MIA injection.

### Tactile Allodynia

Mechanical sensitivity thresholds were determined using von Frey filaments and the updown method. Filaments were applied to the plantar hind paw, bending slightly upon contact. A withdrawal response, often accompanied by paw licking or body movement, was recorded as positive. Eight calibrated von Frey filaments were used, ranging from 0.25 g to 15 g. A paw withdrawal led to testing with a lower-force filament, while no response prompted testing with a higher-force filament. If the strongest filament (15 g) failed to elicit a response, the threshold was recorded as 15 g. Both hind paws were tested with a 5-minute interval between trials.

### Cold Allodynia

Cold sensitivity was assessed by counting foot withdrawal responses following the application of an acetone drop to the plantar surface. While uninjured rats typically showed no reaction, injured rats exhibited nociceptive behaviors such as paw shaking or biting. Each paw was tested five times, with a 3-5 minutes interval between trials. The response frequency was calculated as a percentage.

### Knee Bend Test

A modified version of the ankle-bend nociception test for monoarthritic rats was used to evaluate knee joint sensitivity during normal movement. Rats were gently restrained to minimize movement while ensuring access to both hind limbs. The experimenter manually flexed and extended each knee joint within its natural range of motion while recording the number of vocalizations (squeaks) and struggle responses. Reactions were scored as follows: 0 for no response, 0.5 for struggle only at maximal flexion/extension, 1 for struggle at moderate flexion/extension or vocalization at maximal flexion/extension, and 2 for squeaking in response to moderate flexion/extension. Each test consisted of five flexions and five extensions, and the total knee-bend score (maximum of 20) was used to assess nociceptive sensitivity.

### Cytochemical Staining

Knee joints were harvested by excising the femur and tibia/fibula 1 cm above and below the joint line, dissecting soft tissues, and fixing specimens in 10% neutral-buffered formalin (Sigma-Aldrich) for 14 days. Joints were decalcified, sagittally sectioned, embedded in paraffin, and cut into 4 μm serial sections.

Masson's trichrome staining evaluated collagen composition. Microscopic imaging (Leica DMi8, ×10 objective, Leica X software) was performed on three knees per condition across four fields per knee. Quantitative analysis was conducted using Fiji/ImageJ (v2.14.0).

### PRG4 (Lubricin) Immunolocalization

For PRG4 immunofluorescence, sections were permeabilized with PBS+0.2% Triton ×100 (20 min), blocked (PBS+0.1% Triton X-100+1% BSA, 1 h), and incubated overnight at 4° C. with mouse anti-rat PRG4 monoclonal antibody (Sigma, 1:500). After washing (PBS+0.01% Triton X-100), sections were incubated with Alexa Fluor647-conjugated goat anti-mouse IgG (Thermo Fisher) for 2 h. Controls received secondary antibody only. Sections were counterstained with DAPI (Invitrogen). Microscopic imaging (Leica DMi8, ×10 objective, Leica X software) was performed on three knees per condition across four fields per knee. Quantitative analysis was conducted using Fiji/ImageJ (v2.14.0).

### Substance P Immunolocalization

For anti-substance P immunofluorescence staining, tissue sections were first incubated in 1× citrate buffer at 60° C. overnight for antigen retrieval. The following day, sections were permeabilized with 1×PBS containing 0.2% Triton X-100 for 20 minutes at room temperature, then blocked in a buffer composed of 1×PBS, 0.1% Triton X-100, and 10% rabbit serum for 1 hour at room temperature. Between each step, sections were washed with 1×PBS. Sections were then incubated overnight at 4° C. with rabbit anti-rat substance P polyclonal antibody diluted 1:100 in blocking buffer. After washing with 1×PBS containing 0.01% Triton X-100, sections were incubated for 1 hour at room temperature with an Alexa Fluor 594-conjugated goat antirabbit IgG secondary antibody (Thermo Fisher Scientific). Control sections were treated with secondary antibody only. Finally, all sections were rinsed with 1×PBS and mounted using ProLong Gold Antifade Reagent with DAPI (Invitrogen). Microscopic imaging (Leica DMi8, ×10 objective, Leica X software) was performed on three knees per condition across four fields per knee. Quantitative analysis was conducted using Fiji/ImageJ (v2.14.0).

### Statistical Analysis

Kolmogorov-Smirnov normality test was used to assess normal distribution of values. For multiple comparisons one-way or two-way ANOVA were used at GraphPad Prism v7.03 (GraphPad Software, San Diego, CA) with statistical significance p<0.05.

### Results and Discussion

aCGRP IFP-MSC EV LncRNAs Cargo

Long noncoding RNAs (lncRNAs) are emerging as key epigenetic regulators in skeletal system remodeling, particularly through their influence on cell MSC differentiation. By modulating the balance between osteogenic and adipogenic pathways, lncRNAs play a central role in bone homeostasis, regeneration, and cartilage integrity. Recent studies highlight the complex interplay between lncRNAs and miRNAs, which together coordinate gene expression programs involved in osteoarthritis pathogenesis.

The gene construct of adenovirus vector to transduce IFP-MSC and parental aCGRP+ IFP-MSC cell sorting efficiency, clonogenic capacity (stemness), and immunophenotypic profiling are previously reported. For aCGRP IFP-MSC EVs, the biophysical and biochemical characterization using flow cytometry and nanoparticle tracking analysis were previously described.

In the present application, out of the 96 LncRNAs investigated, 24 unique LncRNAs were detected in the aCGRP EVs. Their levels of relative expression ranged from 0.88 for GAS5 to 0.001106 for LINC000853 (FIGS. 19A-19B). Pathway analysis revealed that most detected LncRNAs are involved in important cell processes such as regulation of stem cell, cell cycle, cell proliferation, inflammation, bone regeneration, osteogenesis, and cell death. However, 7 LncRNAs showed no specific regulatory effects to known miRNAs related to OA pathogenesis.

Specifically, 11 LncRNAs (ZFAS1, EMX2OS, HOTAIRM1, RPS6KA2-AS1, DANCR, LINC-ROR, GACAT1, GNAS-AS1, HAR1A, OIP5-AS1, TERC) interact with miRNAs that promote cell proliferation, prevent apoptosis, and preserve homeostasis. Firstly, ZFAS1 LncRNA has been shown to protect chondrocytes from apoptosis and extracellular matrix degradation by sponging miR-7-5p to regulate FLRT2 expression. EMX2OS LncRNA has been known to interact with miR-653-5p and decrease its effects on cartilage homeostasis. Specifically, miR-653-5p is known for playing a significant role in OA by modulating chondrocyte senescence. In another study, the downregulation of HOTAIRM1LncRNA inhibited MSC viability, induced apoptosis, and suppressed differentiation. This could be the result of HOTAIRM1regulating the miR-125b/BMPR2 axis. RPS6KA2-AS1 LncRNA is associated with gene RPS6KA2 which is involved in chondrocyte differentiation and cartilage formation. Increased RSPS6KA could promote MSC proliferation and chondrogenic differentiation. Importantly, DANCR LncRNA with miR-320a can induce proliferation and chondrogenesis by regulating the Wnt/β-catenin signaling pathway and inhibiting CTNNB1 gene expression during osteogenic differentiation. LINC-ROR LncRNA has been shown to miR-138 and miR-145, and as a result induce SOX9 gene expression, MSC chondrogenesis, cartilage formation. The inhibition of GACAT1 LncRNA has been shown to inhibit the proliferation and migration of oral squamous carcinoma cells suggesting that GACAT1 plays a role in cell proliferation. Similarly, when GNAS-AS1 LncRNA was inhibited in an osteosarcoma study, cell proliferation migration and invasion were also inhibited. Next HAR1A LncRNA has been shown to have immunomodulatory effects by binding to miR-149-3p and suppressing its expression. OIP5-AS1 LncRNA interacts with miR-29b-3p which promotes chondrocyte proliferation and migration and inhibits apoptosis and inflammation. And lastly, TERC LncRNA show a role in bone metabolism by absorbing miRNA-217 which in turn upregulates RUNX2 gene expression.

In addition, 6 LncRNAs (GAS5, TSIX, PCGEM, H19, HOTAIR, SNHG16) have been linked to miRNA regulation that prevent cell proliferation, promote apoptosis, and dysregulate cell homeostasis A study found that GAS5 LncRNA is upregulated in OA patients, and as a result inducing chondrocyte apoptosis and inhibiting chondrocyte proliferation through downregulating miR-137. Also, TSIX LncRNA can be upregulated in OA patients suggesting a role in chondrocyte dysfunction by interacting with miR-320a. Inhibiting TSIX LncRNA lead to enhanced cell viability and mitigated IL-1B-induced apoptosis. A study found that PCGEM LncRNA is upregulated in synoviocytes of OA patients. Specifically, PCGEM LncRNA interacts with miR-142-5p resulting in the upregulation of RUNX2 gene expression. Next H19 LncRNA was shown to interact with miR-130a. H19 LncRNA knock-out resulted in increased viability, decreased apoptosis and reduced inflammatory factor secretion. HOTAIR LncRNA was shown to have an antagonistic relationship with miR-17-5p and upon HOTAIR upregulation miR-17-5p expression is downregulated leading to increased apoptosis. Lastly, SNHG16 LncRNA was shown to interact with miR-373-3p. SNHG16 LncRNA knock-out result in an increase in miR-373-3p which led to decreased IL-1B induced apoptosis, which implies that SNHG16 regulates miR-373-3p.

aCGRP IFP-MSC EVs Effects on Dorsal Root Ganglia Under Inflammatory Conditions

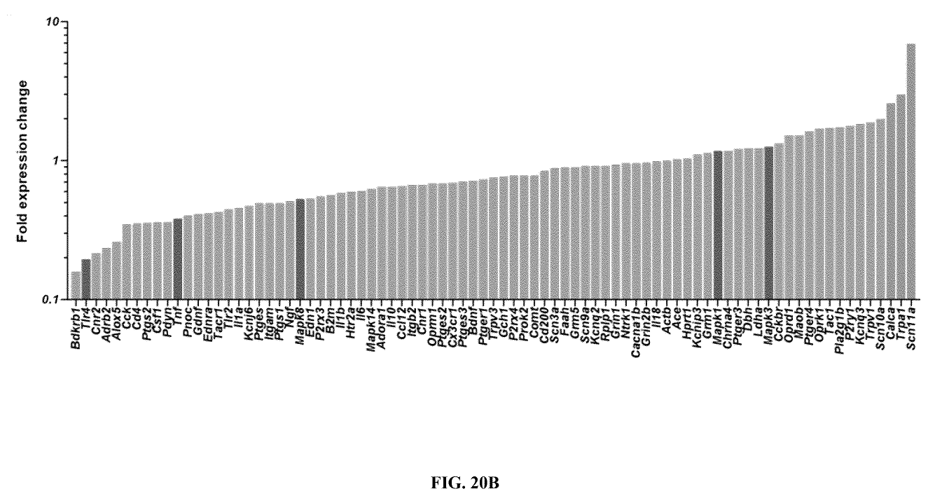

Preliminary studies have shown that CGRP 8-37 (truncated CGRP peptide) acts as a CGRP antagonist temporarily reverse migraine and neuropathic pain symptoms in animal models. Specifically, AAV_CGRP₈₋₃₇ intraspinal or intrathecal injection can decrease chronic neuropathic pain in rat spinal cord injury and peripheral nerve injury pain models. Herein, DRGs showed identical morphology with and without the addition of aCGRP IFP-MSC EVs treatment (FIG. 20A). TIC-stimulated DRGs molecular profiling indicated an overall reduced neuroinflammatory profile upon exposure to aCGRP IFP-MSC EVs (FIG. 20B). Out of the 84 genes investigated, only 3 (Calca, Trpa1, and Scn11a) showed increased expression (>2 fold) compared to TIC-stimulated DRGs alone. Interestingly Calca, which is the gene for CGRP, is a classic marker of nociceptive DRGs. The high Calca gene expression levels could be the result of aCGRP being recognized as CGRP by qPCR assay. The transient receptor potential ankyrin 1 (TRPA1) has shown that is involved in inflammatory responses, and in the conversion of physical and chemical stimuli in irritative or pain sensations. Lastly, Scn11a encodes the tetrodotoxin-resistant (TTX-R) sodium channel Nav1.9a/NaN, which is preferentially expressed in nociceptive primary sensory neurons of DRGs.

Importantly, five important genes (Mapk1, Mapk3, Mapk8, Tlr4, and Tnf) involved in neuroinflammation were highly down-regulated (>2-fold) upon exposure to aCGRP IFPMSC EVs. In this context, mitogen-activated protein kinases (MAPKs) are involved in inflammation and modulate nociceptive signaling and the peripheral/central sensitization, whereas their inhibition results in anti-inflammatory effects in inflammatory diseases. Different MAPKs isoforms, such as MAPK1 and MAPK8, contribute to pain sensitization via both neuronal and glial mechanisms. These two isoforms were downregulated following EVs treatment (FIG. 20B). MAPK8 activation in astrocytes contributes to the maintenance of chronic pain. MAPK1 can induce alone inflammatory pain whereas with MAPK3 results in sensory neuron survival. Also, TLR4 is a pattern recognition receptor and its activation on spinal microglia and astrocytes leads to the production of pro-inflammatory cytokines, including TNF, and contributes to the development of pain. TLR4 activation has been shown to induce tactile allodynia through the release of prostaglandin E2 (PGE2) and TNF, which are critical mediators of pain signaling. Additionally, TLR4 activation enhances calcium signaling in astrocytes, promoting cytokine production and further promotion of neuroinflammation and pain. Finally, TNF is a pro-inflammatory cytokine that plays a pivotal role in the pathogenesis of neuroinflammation and pain. TNF is produced by activated glial cells in response to TLR4 activation and contributes to the sensitization of nociceptive pathways. TNF-dependent pathways are involved in the maintenance of persistent pain states, highlighting its importance in chronic pain conditions.

Effects of aCGRP IFP-MSC EVs on Chondrocytes Under Inflammatory Conditions

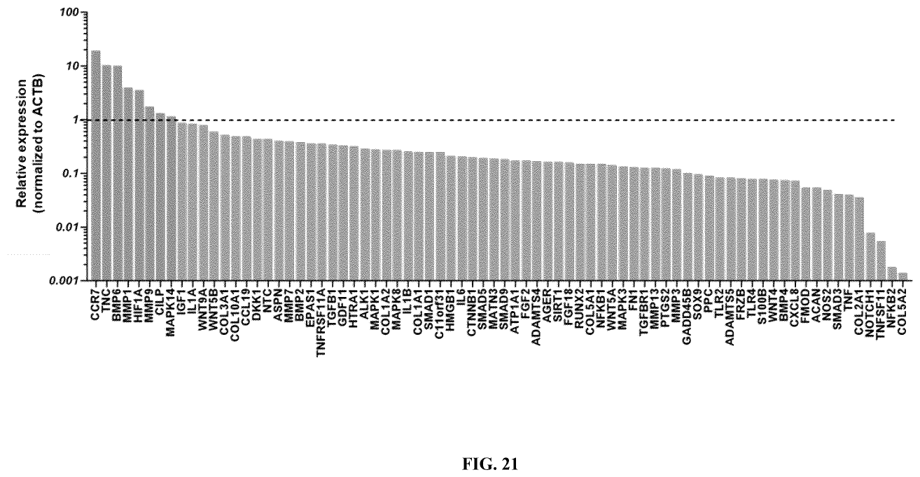

Treatment of chondropellets/synoviocytes inflammatory co-cultures with aCGRP IFP-MSC EVs led to upregulated expression of several genes in chondropellets with diverse roles in cartilage biology, some of which are closely tied to anabolic processes, while others are typically associated with catabolic or inflammatory functions (FIG. 21). Notably, the upregulation of BMP6 and HIF1A genes expression suggests a pro-anabolic effect of the EVs treatment. BMP6, a member of the TGF-β superfamily, is well recognized for its ability to stimulate chondrogenesis, promote extracellular matrix (ECM) synthesis, and enhance the expression of key anabolic markers such as aggrecan and collagen type II. Similarly, HIFIA is a master regulator of cellular adaptation to hypoxia, a condition inherent to the avascular cartilage environment. HIFIA supports anabolic activity by promoting chondrocyte survival and upregulating SOX9, a critical transcription factor for cartilage matrix synthesis. These gene expression changes suggest that aCGRP IFP-MSC EVs may create a microenvironment conducive to cartilage repair.

Interestingly, the EVs-induced upregulation of TNC (Tenascin-C) gene expression may also play a supportive role in anabolism, particularly in the early stages of cartilage remodeling. TNC is known to be upregulated during tissue injury and repair and can enhance cell migration and adhesion. However, it also has context-dependent effects, as sustained TNC expression may contribute to inflammation through Toll-like receptor activation, potentially limiting its long-term anabolic benefits. Likewise, Cartilage Intermediate Layer Protein (CILP) has been shown to inhibit insulin-like growth factor-1 (IGF-1) signaling, which is crucial for anabolic responses in cartilage. While its expression is part of normal ECM composition, high levels could dampen IGF-1-mediated repair.

More surprising was the upregulation of MMP1 and MMP9 genes expression, which traditionally is associated with matrix degradation (FIG. 21). These metalloproteinases are commonly elevated in OA tissue and contribute to cartilage breakdown by degrading collagens and other ECM components. However, transient or controlled expression of MMPs may be necessary for matrix remodeling and the removal of damaged ECM components prior to repair, particularly during early phases of regeneration. Their induction by EVs might therefore reflect a preparatory remodeling response rather than overt degeneration. Similarly, MAPK14 (p38 MAPK) gene expression exhibits a dual role in chondrocyte biology, where it can support anabolic activity by stabilizing and upregulating SOX9 under certain conditions, such as TGF-β stimulation, but also promote catabolic responses through inflammatory signaling and chondrocyte apoptosis in other contexts.

Lastly, the observed upregulation of CCR 7 gene expression, a chemokine receptor typically associated with immune cell recruitment and inflammation, raises important considerations. While traditionally viewed as a contributor to synovial inflammation in OA, recent evidence suggests that chemokine signaling can also participate in tissue repair by modulating cell migration and intercellular communication. Its role in EVs-mediated cartilage regeneration may reflect the broader immunomodulatory effects of EVs on the joint environment.

Taken together, the EVs-induced gene expression profile suggests a coordinated response that may initially involve ECM remodeling and immune modulation, followed by activation of anabolic pathways critical for cartilage repair. While some upregulated genes are associated with catabolic or inflammatory functions in chronic OA, their controlled activation in vitro may reflect a regenerative rather than degenerative program. The therapeutic potential of EVs could also be further optimized through preconditioning/priming in vitro strategies. For example, studies showed that priming parental MSC with oxytocin and inflammatory cues significantly enrich EVs miRNAs cargo resulting in upregulation of FMOD, TIMP1, CXCL8, SOX9, IGF1, and BMP4 genes expression in chondrocytes linked to cartilage matrix remodeling and chondroprotection. These changes are accompanied by enhanced activation of pathways related to connective tissue development and extracellular matrix organization.

aCGRP IFP-MSC EVs Effect on Pain Sensitization Assessed by Behavior Testing

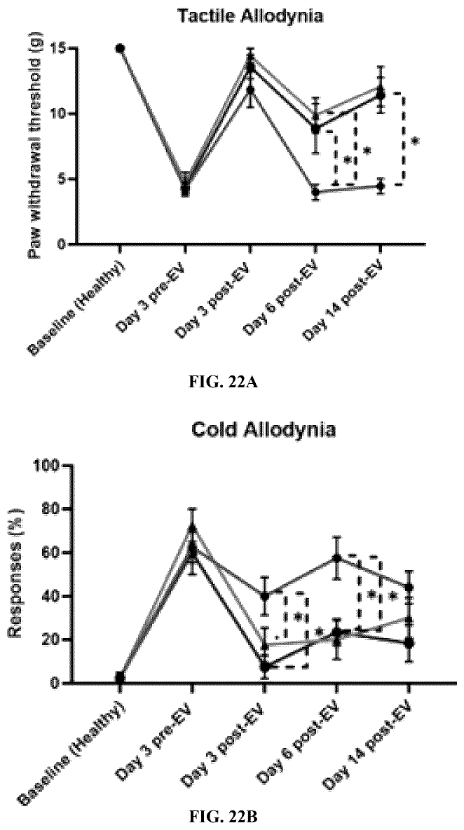

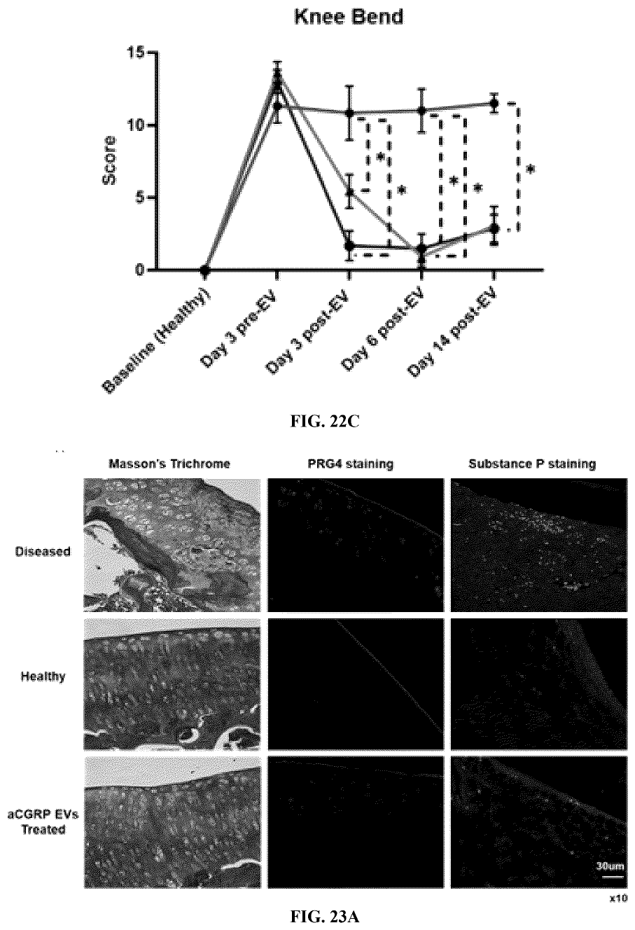

The behavioral outcomes observed following MIA injection support the development of an inflammatory arthritis phenotype characterized by joint inflammation, cartilage degradation, and persistent pain. MIA induces chondrocyte death and disrupts cartilage metabolism, mimicking the degenerative processes seen in human OA. In this model, non-treated animals exhibited heightened pain sensitivity across multiple modalities (FIGS. 22A-22C). The acetone test revealed increased cold allodynia, the knee-bend test demonstrated enhanced joint-related pain responses, and the cold hypersensitivity assay showed decreased paw withdrawal thresholds (marked with lines). All behavioral tests reflect the development of central and peripheral sensitization that typically accompanies chronic joint degeneration.

Remarkably, animals treated with EVs displayed attenuated pain behaviors across all tests. aCGRP IFP-MSC EVs-treated knees showed a significantly reduced responsive-ness to acetone application, indicating a decrease in cold sensitivity. Significant differences in pain withdrawal threshold existed in von Frey filaments testing six days after EV treatment and persisted at the last timepoint fourteen days after EV treatment. Meanwhile, the EV treated groups had significantly reduced knee bend scores starting three days after EV treatment and lasting until the final timepoint. Lower pain scores in the knee-bend test further indicate reduced mechanical joint pain, and the improved withdrawal thresholds in the von Frey tactile test highlight a broader analgesic effect. These findings indicate that EV therapy may counteract key pathophysiological features of OA, such as neuroinflammation and aberrant nociceptive signaling. Given that pain is one of the most debilitating features of OA, the ability of EVs to modulate these behavioral responses underscores their potential as a disease-modifying therapeutic, capable not only of addressing inflammation and tissue repair but also mitigating the chronic pain associated with joint injury.

aCGRP IFP-MSC EVs Effects on Cartilage Homeostasis and Pain Signaling Assessed by Histological Evaluation

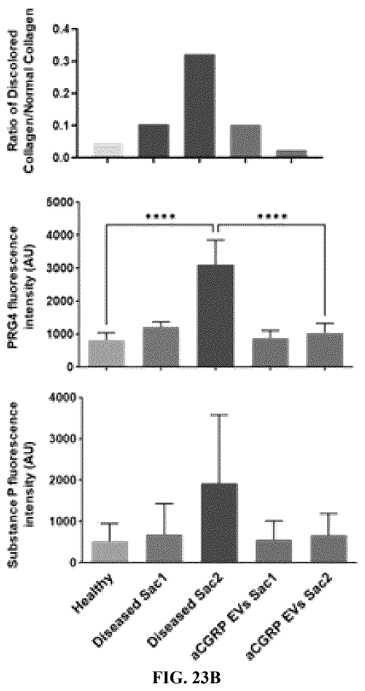

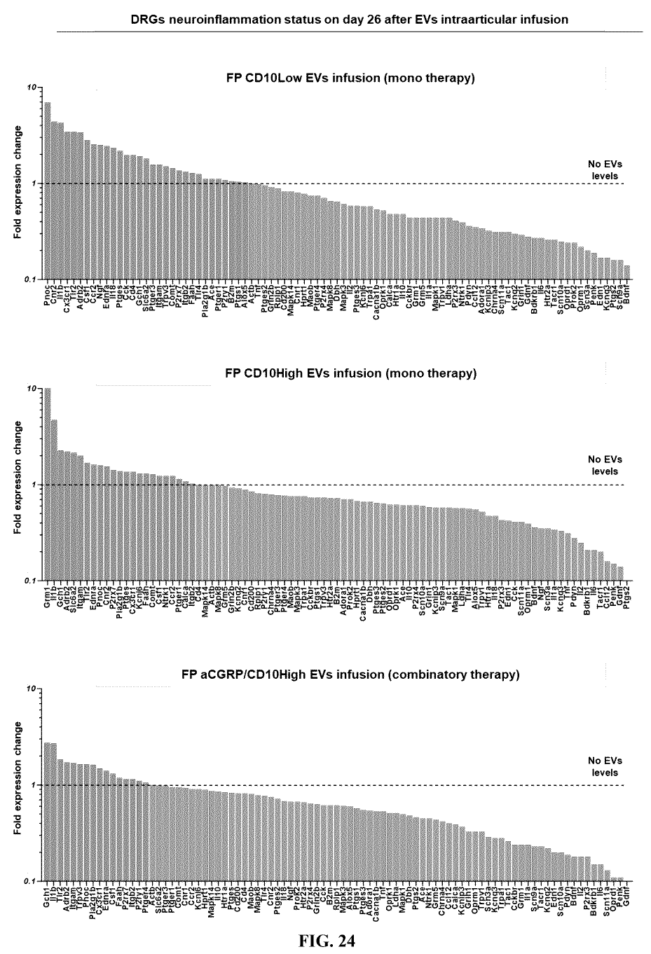

Histochemical and immunohistochemical evaluation revealed chondroprotective and analgesic effects upon aCGRP IFP-MSC EVs intra-articular administration in rat knee joint (FIGS. 23A-23B). Masson's trichrome staining has been effectively employed as a histochemical technique to assess alterations in collagen composition. Cartilage from the disease group exhibited pronounced staining, indicating collagen disruption, when compared to other experimental groups. Notably, it appears to be progression of the disease in untreated group from the timepoint of the first sacrifice to the timepoint of the second as the ratio of discolored to normal cartilage increases. Remarkably, administration of aCGRP IFPMSC EVs led to a significant enhancement in normal collagen content with a lower ratio of discolored to normal collagen, closely resembling staining of the healthy control group (FIGS. 23A-23B).

To explore the phenotypic impact of aCGRP IFP-MSC EVs administration, immunohistochemical analysis of PRG4 expression in situ was conducted (FIGS. 23A-23B). In healthy cartilage, PRG4+ cartilage-derived stem/progenitor cells (CSPCs) formed a well-defined band in the superficial layer. In contrast, diseased cartilage exhibited some PRG4 expression in this zone, coupled with a significant upregulation in the intermediate zone, where chondrocytes likely increase PRG4 production to compensate for superficial layer loss during disease progression. Importantly, the expression of PRG4 in the aCGRP IFP-MSC EVs-treated group was comparable to that of the healthy group with no significant difference between the healthy and treated groups at the second timepoint sacrifice.

Finally, to analyze the effects of aCGRP IFP-MSC EVs administration on SP degradation an immunofluorescence test was performed on synovium/IFP tissues (FIGS. 23A-23B). Using healthy tissue as a baseline the SP presence of the diseased samples and the EVs-treated samples at different end points in the experiment were compared. aCGRP IFP-MSC EVs-treated group showed decreased SP presence in synovium/IFP tissues. Moreover, SP not only plays a key role in nociception but also modulates local neurogenic inflammation and immune responses. It increases vascular permeability to promote immune cell infiltration and influences macrophage migration and polarization at sites of inflammation. Given that aCGRP IFP-MSC EVs are derived from parental MSC that highly express CD10 (neprilysin), an endopeptidase that cleaves Substance P, decreased SP presence in the joint capsule is expected.

These findings provide for the therapeutic use of aCGRP IFPMSC EVs in the context of OA. OA is characterized by cartilage matrix disruption, chronic inflammation, and failed repair responses due to limited regenerative capacity of the joint. The observed restoration of collagen content and structure following EVs treatment, as shown by Masson's trichrome staining, indicates a protective or reparative effect on extracellular matrix integrity, a critical component often compromised in OA. Furthermore, the preservation and relocalization of PRG4+ CSPCs in the superficial zone implies that EVs may help maintain the endogenous repair machinery disrupted in disease. Also, the decreased SP presence in EVs-treated group, along with the known role of SP in promoting neurogenic inflammation and macrophage recruitment, supports the hypothesis that neprilysin-expressing EVs could attenuate pain cascades associated with OA. Together, these results highlight a multifaceted mechanism by which aCGRP IFP-MSC EVs may mitigate joint degeneration.

### Conclusions

Clinically, nonoperative treatment for osteoarthritis (OA) typically progresses from lifestyle modification to oral and topical NSAIDs, to intra-articular injections. NSAIDs remain a first-line therapy due to their consistent efficacy in reducing pain and improving function, with both oral and topical forms strongly supported by AAOS and ACR guidelines. However, their long-term use is limited by gastrointestinal, renal, and cardiovascular risks. When oral medications fail, intra-articular corticosteroid injections are often the next step, providing meaningful but short-term relief-typically lasting a few weeks to months. They are effective for inflammatory flares, yet concerns remain about potential cartilage damage with frequent use and systemic side effects such as transient hyperglycemia. Hyaluronic acid injections, aim to restore joint lubrication and viscoelasticity but show inconsistent efficacy across studies, and both AAOS and ACR generally discourage their routine use due to limited and variable evidence. Platelet-rich plasma (PRP) therapy has emerged as a promising biologic option, with several randomized trials and meta-analyses suggesting superior symptom improvement compared with saline or hyaluronic acid, particularly in earlier stages of OA. Still, the lack of standardization in PRP preparation, inconsistent results, and limited insurance coverage temper its widespread adoption, leading AAOS to issue only a limited recommendation. MSC and bone-marrow aspirate concentrate (BMAC) injections represent another frontier, targeting biologic repair and anti-inflammatory modulation, yet evidence remains low-quality and heterogeneous, with uncertain long-term safety and high cost—thus, major societies recommend use only within clinical trials. Across all injection therapies, the consistent limitation is durability—most provide transient symptom relief without altering disease progression. Emerging work on EVs-based therapeutics shows the potential to deliver sustained anti-inflammatory and immunomodulatory effects, offering a more durable, disease-modifying approach.

Under regulatory-compliant conditions, EVs genetically modified with the CGRP antagonist IFP-MSC have been successfully generated. Specifically, it has been shown that aCGRP

IFP-MSC EVs exert potent immunomodulatory, analgesic, and chondroprotective effects in an animal model of OA. In stimulated DRGs, EVs downregulated key mediators of neuroinflammation and nociceptive sensitization, including MAPK1/8, TLR4, and TNF, indicating their role in dampening chronic pain signaling pathways. Behaviorally, aCGRP IFP-MSC EVs-treated animals exhibited marked reductions in pain behaviors reflecting functional improvements consistent with reduced peripheral and central sensitization. Histologically, aCGRP IFP-MSC EVs administration improved cartilage matrix integrity, restored superficial zone CSPCs organization, and showed reduced SP levels. Collectively, these findings provide for the therapeutic use of aCGRP IFP-MSC EVs as a multi-modal intervention capable of addressing inflammation, pain, and structural degradation in OA, highlighting their use as a disease-modifying treatment.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### Tables

TABLE 1
Human MSC exosome miRNA array

| qPCR Primer | Mature miRNA name | Mature miRNA accession |
|---|---|---|
| HmiRQP0002 | hsa-let-7a-5p | MIMAT0000062 |
| HmiRQP0004 | hsa-let-7b-5p | MIMAT0000063 |
| HmiRQP0007 | hsa-let-7d-5p | MIMAT0000065 |
| HmiRQP0010 | hsa-let-7e-5p | MIMAT0000066 |
| HmiRQP0012 | hsa-let-7f-5p | MIMAT0000067 |
| HmiRQP0015 | hsa-let-7g-5p | MIMAT0000414 |
| HmiRQP0017 | hsa-let-7i-5p | MIMAT0000415 |
| HmiRQP0018 | hsa-miR-100-5p | MIMAT0000098 |
| HmiRQP0029 | hsa-miR-106b-5p | MIMAT0000680 |
| HmiRQP0030 | hsa-miR-107 | MIMAT0000104 |
| HmiRQP0031 | hsa-miR-10a | MIMAT0004555 |
| HmiRQP0032 | hsa-miR-10a-5p | MIMAT0000253 |
| HmiRQP0034 | hsa-miR-10b-5p | MIMAT0000254 |
| HmiRQP3286 | hsa-miR-1185-2-3p | MIMAT0022713 |
| HmiRQP0056 | hsa-miR-122-5p | MIMAT0000421 |
| HmiRQP0078 | hsa-miR-1246 | MIMAT0005898 |
| HmiRQP0087 | hsa-miR-1255a | MIMAT0005906 |
| HmiRQP0093 | hsa-miR-125a | MIMAT0004602 |
| HmiRQP0094 | hsa-miR-125a-5p | MIMAT0000443 |
| HmiRQP0096 | hsa-miR-125b-5p | MIMAT0000423 |
| HmiRQP0099 | hsa-miR-126-3p | MIMAT0000445 |
| HmiRQP0111 | hsa-miR-127-3p | MIMAT0000446 |
| HmiRQP0134 | hsa-miR-1290 | MIMAT0005880 |
| HmiRQP0152 | hsa-miR-1305 | MIMAT0005893 |
| HmiRQP0156 | hsa-miR-130a-3p | MIMAT0000425 |
| HmiRQP0160 | hsa-miR-132 | MIMAT0004594 |
| HmiRQP0161 | hsa-miR-132-3p | MIMAT0000426 |
| HmiRQP0173 | hsa-miR-136-5p | MIMAT0000448 |
| HmiRQP0175 | hsa-miR-137 | MIMAT0000429 |
| HmiRQP0181 | hsa-miR-140-5p | MIMAT0000431 |
| HmiRQP0186 | hsa-miR-142-3p | MIMAT0000434 |
| HmiRQP0188 | hsa-miR-143-3p | MIMAT0000435 |
| HmiRQP0190 | hsa-miR-144-3p | MIMAT0000436 |
| HmiRQP0192 | hsa-miR-145-5p | MIMAT0000437 |
| HmiRQP0195 | hsa-miR-146a | MIMAT0004608 |
| HmiRQP0196 | hsa-miR-146a-5p | MIMAT0000449 |
| HmiRQP0204 | hsa-miR-148a-3p | MIMAT0000243 |
| HmiRQP0206 | hsa-miR-148b-3p | MIMAT0000759 |
| HmiRQP0208 | hsa-miR-149-5p | MIMAT0000450 |
| HmiRQP0210 | hsa-miR-150-5p | MIMAT0000451 |
| HmiRQP0211 | hsa-miR-151a-3p | MIMAT0000757 |
| HmiRQP0219 | hsa-miR-154-5p | MIMAT0000452 |
| HmiRQP0222 | hsa-miR-15a | MIMAT0004488 |
| HmiRQP0223 | hsa-miR-15a-5p | MIMAT0000068 |
| HmiRQP0225 | hsa-miR-15b-5p | MIMAT0000417 |
| HmiRQP0227 | hsa-miR-16-5p | MIMAT0000069 |
| HmiRQP0233 | hsa-miR-181a-2-3p | MIMAT0004558 |
| HmiRQP0231 | hsa-miR-181a-3p | MIMAT0000270 |
| HmiRQP0232 | hsa-miR-181a-5p | MIMAT0000256 |
| HmiRQP0238 | hsa-miR-182-3p | MIMAT0000260 |
| HmiRQP0245 | hsa-miR-184 | MIMAT0000454 |
| HmiRQP0247 | hsa-miR-185-5p | MIMAT0000455 |
| HmiRQP0251 | hsa-miR-187-3p | MIMAT0000262 |
| HmiRQP0255 | hsa-miR-18a-5p | MIMAT0000072 |
| HmiRQP0263 | hsa-miR-191-5p | MIMAT0000440 |
| HmiRQP0277 | hsa-miR-193a-5p | MIMAT0004614 |
| HmiRQP0279 | hsa-miR-193b-5p | MIMAT0004767 |
| HmiRQP0284 | hsa-miR-196a-5p | MIMAT0000226 |
| HmiRQP0286 | hsa-miR-196b-5p | MIMAT0001080 |
| HmiRQP3038 | hsa-miR-197-5p | MIMAT0022691 |
| HmiRQP0289 | hsa-miR-199a-3p | MIMAT0000232 |
| HmiRQP0290 | hsa-miR-199a-5p | MIMAT0000231 |
| HmiRQP0289 | hsa-miR-199b-3p | MIMAT0004563 |
| HmiRQP0291 | hsa-miR-199b-5p | MIMAT0000263 |
| HmiRQP0293 | hsa-miR-19a-3p | MIMAT0000073 |
| HmiRQP0295 | hsa-miR-19b-3p | MIMAT0000074 |
| HmiRQP0298 | hsa-miR-200a-3p | MIMAT0000682 |
| HmiRQP0307 | hsa-miR-205-5p | MIMAT0000266 |
| HmiRQP0312 | hsa-miR-20a-5p | MIMAT0000075 |
| HmiRQP0314 | hsa-miR-20b-5p | MIMAT0001413 |
| HmiRQP0320 | hsa-miR-214-3p | MIMAT0000271 |
| HmiRQP0316 | hsa-miR-21-5p | MIMAT0000076 |
| HmiRQP0327 | hsa-miR-218-5p | MIMAT0000275 |
| HmiRQP0338 | hsa-miR-221-3p | MIMAT0000278 |
| HmiRQP0339 | hsa-miR-222 | MIMAT0000279 |
| HmiRQP0339 | hsa-miR-222-3p | MIMAT0000279 |
| HmiRQP0342 | hsa-miR-223-3p | MIMAT0000280 |
| HmiRQP0332 | hsa-miR-22-3p | MIMAT0000077 |
| HmiRQP0344 | hsa-miR-23a-3p | MIMAT0000078 |
| HmiRQP0346 | hsa-miR-23b-3p | MIMAT0000418 |
| HmiRQP0349 | hsa-miR-24-3p | MIMAT0000080 |
| HmiRQP0352 | hsa-miR-25-3p | MIMAT0000081 |
| HmiRQP0354 | hsa-miR-26a-5p | MIMAT0000082 |
| HmiRQP0357 | hsa-miR-26b-5p | MIMAT0000083 |
| HmiRQP0361 | hsa-miR-27b-3p | MIMAT0000419 |
| HmiRQP0362 | hsa-miR-28-5p | MIMAT0000085 |
| HmiRQP0364 | hsa-miR-296-5p | MIMAT0000690 |
| HmiRQP0369 | hsa-miR-299-5p | MIMAT0002890 |
| HmiRQP0371 | hsa-miR-29a-3p | MIMAT0000086 |
| HmiRQP0373 | hsa-miR-29b-3p | MIMAT0000100 |
| HmiRQP0375 | hsa-miR-29c-3p | MIMAT0000681 |
| HmiRQP0378 | hsa-miR-301a-3p | MIMAT0000688 |
| HmiRQP1518 | hsa-miR-3065-5p | MIMAT0015066 |
| HmiRQP0391 | hsa-miR-30a-5p | MIMAT0000087 |
| HmiRQP0398 | hsa-miR-30d-5p | MIMAT0000245 |
| HmiRQP0400 | hsa-miR-30e-5p | MIMAT0000692 |
| HmiRQP1523 | hsa-miR-320e | MIMAT0015072 |
| HmiRQP0410 | hsa-miR-323a-3p | MIMAT0000755 |
| HmiRQP0404 | hsa-miR-32-5p | MIMAT0000090 |
| HmiRQP0421 | hsa-miR-335-5p | MIMAT0000765 |
| HmiRQP0423 | hsa-miR-337-3p | MIMAT0000754 |
| HmiRQP0424 | hsa-miR-337-5p | MIMAT0004695 |
| HmiRQP0436 | hsa-miR-342-3p | MIMAT0000753 |
| HmiRQP0440 | hsa-miR-34a-5p | MIMAT0000255 |
| HmiRQP1907 | hsa-miR-3613-3p | MIMAT0017991 |
| HmiRQP0446 | hsa-miR-361-5p | MIMAT0000703 |
| HmiRQP0448 | hsa-miR-362-5p | MIMAT0000705 |
| HmiRQP0449 | hsa-miR-363-3p | MIMAT0000707 |
| HmiRQP0451 | hsa-miR-365a-3p | MIMAT0000710 |
| HmiRQP1944 | hsa-miR-3665 | MIMAT0018087 |
| HmiRQP0454 | hsa-miR-369-3p | MIMAT0000721 |
| HmiRQP0463 | hsa-miR-374a-5p | MIMAT0000727 |
| HmiRQP0464 | hsa-miR-374b-5p | MIMAT0004955 |
| HmiRQP0467 | hsa-miR-376a-3p | MIMAT0000729 |
| HmiRQP0472 | hsa-miR-377-3p | MIMAT0000730 |
| HmiRQP2046 | hsa-miR-378g | MIMAT0018937 |
| HmiRQP2182 | hsa-miR-378i | MIMAT0019074 |
| HmiRQP0476 | hsa-miR-379-5p | MIMAT0000733 |
| HmiRQP0480 | hsa-miR-382-5p | MIMAT0000737 |
| HmiRQP0484 | hsa-miR-409-3p | MIMAT0001639 |
| HmiRQP0487 | hsa-miR-411-3p | MIMAT0004813 |
| HmiRQP0486 | hsa-miR-411-5p | MIMAT0003329 |
| HmiRQP0492 | hsa-miR-423-5p | MIMAT0004748 |
| HmiRQP0494 | hsa-miR-424-5p | MIMAT0001341 |
| HmiRQP1612 | hsa-miR-4286 | MIMAT0016916 |
| HmiRQP0501 | hsa-miR-432-5p | MIMAT0002814 |
| HmiRQP2084 | hsa-miR-4454 | MIMAT0018976 |
| HmiRQP2100 | hsa-miR-4466 | MIMAT0018993 |
| HmiRQP0503 | hsa-miR-448 | MIMAT0001532 |
| HmiRQP2128 | hsa-miR-4488 | MIMAT0019022 |
| HmiRQP0506 | hsa-miR-450a-5p | MIMAT0001545 |
| HmiRQP2159 | hsa-miR-4516 | MIMAT0019053 |
| HmiRQP0509 | hsa-miR-451a | MIMAT0001631 |
| HmiRQP0516 | hsa-miR-455-5p | MIMAT0003150 |
| HmiRQP2241 | hsa-miR-4647 | MIMAT0019709 |
| HmiRQP2495 | hsa-miR-4792 | MIMAT0019964 |
| HmiRQP0540 | hsa-miR-497-5p | MIMAT0002820 |
| HmiRQP0543 | hsa-miR-499a-5p | MIMAT0002870 |
| HmiRQP1537 | hsa-miR-514b-5p | MIMAT0015087 |
| HmiRQP0610 | hsa-miR-525-5p | MIMAT0002838 |
| HmiRQP0616 | hsa-miR-532-3p | MIMAT0004780 |
| HmiRQP0622 | hsa-miR-543 | MIMAT0004954 |
| HmiRQP0627 | hsa-miR-548a-5p | MIMAT0004803 |
| HmiRQP2631 | hsa-miR-548ar-3p | MIMAT0022266 |
| HmiRQP2015 | hsa-miR-548y | MIMAT0018354 |
| HmiRQP0673 | hsa-miR-574-3p | MIMAT0003239 |
| HmiRQP0674 | hsa-miR-574-5p | MIMAT0004795 |
| HmiRQP0675 | hsa-miR-575 | MIMAT0003240 |
| HmiRQP0684 | hsa-miR-582-5p | MIMAT0003247 |
| HmiRQP2775 | hsa-miR-6089 | MIMAT0023714 |
| HmiRQP0716 | hsa-miR-612 | MIMAT0003280 |
| HmiRQP0720 | hsa-miR-615-3p | MIMAT0003283 |
| HmiRQP0734 | hsa-miR-626 | MIMAT0003295 |
| HmiRQP0740 | hsa-miR-630 | MIMAT0003299 |
| HmiRQP0760 | hsa-miR-650 | MIMAT0003320 |
| HmiRQP0769 | hsa-miR-658 | MIMAT0003336 |
| HmiRQP0786 | hsa-miR-7-5p | MIMAT0000252 |
| HmiRQP0796 | hsa-miR-767-5p | MIMAT0003882 |
| HmiRQP4563 | hsa-miR-7975 | MIMAT0031178 |
| HmiRQP0802 | hsa-miR-802 | MIMAT0004185 |
| HmiRQP0817 | hsa-miR-888-5p | MIMAT0004916 |
| HmiRQP0837 | hsa-miR-93-5p | MIMAT0000093 |
| HmiRQP0853 | hsa-miR-98-5p | MIMAT0000096 |
| HmiRQP0854 | hsa-miR-99a-5p | MIMAT0000097 |
| HmiRQP0856 | hsa-miR-99b | MIMAT0004678 |
| HmiRQP0857 | hsa-miR-99b-5p | MIMAT0000689 |
| HK1(RNU6-2) | HK1(RNU6-2) | NR_002752 |
| HK2(SNORD44) | HK2(SNORD44) | NR_002750 |
| HK3(SNORD48) | HK3(SNORD48) | NR_002745 |
| HK4(SNORD47) | HK4(SNORD47) | NR_002746 |
| HK5(SNORD49A) | HK5(SNORD49A) | NR_002744 |
| HK6(SNORD68) | HK6(SNORD68) | NR_002450 |

TABLE 2
Cytokines and growth factors array
Protein name

| | |
|---|---|
| Eotaxin | AR |
| Eotaxin-2 | βFGF |
| ICAM-1 | βNGF |
| IFN-γ | EGF |
| I-309 | EGF R |
| IL-1a | FGF-4 |
| IL-1β | FGF-6 |
| IL-2 | FGF-7 |
| IL-3 | G-CSF |
| IL-4 | GDNF |
| IL-6 | GM-CSF |
| IL-6 sR | HB-EGF |
| IL-7 | HGF |
| IL8 | IGFBP-1 |
| IL-10 | IGFBP-2 |
| IL-11 | IGFBP-3 |
| IL12-p40 | IGFBP-4 |
| IL12-p70 | IGFBP-6 |
| IL-13 | IGF-I |
| IL-15 | IGF-I SR |
| IL-16 | IGF-II |
| IL17 | M-CSF |
| IP-10 | MCSF R |
| MCP-1 | NT-3 |
| MCP-2 | NT-4 |
| MIG | PDGF Rα |
| MIP-1-α | PDGF Rβ |
| MIP-1-β | PDGF-AA |
| MIP-1-δ | PDGF-AB |
| RANTES | PDGF-BB |
| TNF-α | PIGF |
| TNF-β | SCF |
| STNF-RI | SCF R |
| STNFRII | TGF-α |
| TIMP-2 | TGF-β |
| | TGF-β2 |
| | TGF-β3 |
| | VEGF |
| | VEGF R2 |
| | VEGF R3 |
| | VEGF D |

TABLE 3
Human Macrophage Polarization Marker qPCR Array
Gene name

| | |
|---|---|
| BMP7 | TLR2 |
| HIF1A | NONO |
| IL1B | CD68 |
| MRC1 | HLA-DRB1 |
| STAT6 | IL6 |
| ACTB | PPARG |
| CCL2 | TLR4 |
| HLA-DQA1 | PPIH |
| ILIR1 | CD80 |
| NFKB1 | IFNG |
| TGFB1 | IRF4 |
| GAPDH | SOCS3 |
| CD163 | TLR8 |
| HLA-DQB1 | GDC |
| IL1R2 | CD86 |
| NOS2 | IL10 |
| TLR1 | KLF4 |
| LDHA | STAT1 |
| CD200R1 | TNF |
| HLA-DRA | PPC |
| IL23A | FABP4 |
| PECAM1 | IL12A |
| STAT3 | MMP9 |
| VEGFA | |

TABLE 4
RT2 Profiler neuropathic & inflammatory array
Gene name

| | |
|---|---|
| ACE | MAPK1 |
| ADORA1 | MAPK14 |
| ADRB2 | MAPK3 |
| ALOX5 | MAPK8 |
| BDKRB1 | NGF |
| BDNF | NTRK1 |
| CACBA1B | OPRD1 |
| CALCA | OPRK1 |
| CCK | OPRM1 |
| CCKBR | P2RX3 |
| CCL2 | P2RX4 |
| CCRS | P2RX7 |
| CD200 | P2RXY1 |
| CD4 | POYN |
| CHRN4 | PENK |
| CNR1 | PLA2G1B |
| CNR2 | PNOC |
| COMT | PROK2 |
| CSF1 | PTGER1 |
| CX3CR1 | PTGER3 |
| DBH | PTGER4 |
| EDN1 | PTGES |
| EDNRA | PTGES2 |
| FAAH | PTGES3 |
| GCH1 | PTGS1 |
| GDNF | PTGS2 |
| GRIN1 | SCN10A |
| GRIN2B | SCN11A |
| GRM1 | SCN3A |
| GRM5 | SCN9A |
| HTR1A | SLC6A2 |
| HTR2A | TAC1 |
| IL10 | TACR1 |
| LI18 | TLR2 |
| LI1A | TLR4 |
| IL1B | TNF |
| IL2 | TRPA1 |
| IL6 | TRPV1 |
| ITGAM | TRPV3 |
| ITGB2 | ACTB |
| KCNIP3 | B2M |
| KCNJ6 | GAPDH |
| KCNQ2 | HRPT1 |
| KCNQ3 | RPLPO |
| MAOB | HGDC |

TABLE 5

| | # of | Injection |
|---|---|---|
| Group | animal | Received |
| Diseased | 1 | No injection |
| animals | 2 | No injection |
| (negative | 3 | No injection |
| control) | 4 | No injection |
| | 5 | No injection |
| | 6 | No injection |
| | 7 | No injection |
| | 8 | No injection |
| CD10High | 1 | CD10High |
| EVs-treated | 2 | CD10High |
| animals | 3 | CD10High |
| (positive | 4 | CD10High |
| control) | 5 | CD10High |
| | 6 | CD10High |
| | 7 | CD10High |
| | 8 | CD10High |
| aCGRP | 1 | aCGRP |
| EVs-treated | 2 | aCGRP |
| animals | 3 | aCGRP |
| | 4 | aCGRP |
| | 5 | aCGRP |
| | 6 | aCGRP |
| | 7 | aCGRP |
| | 8 | aCGRP |

1.-80. (canceled)

81. A population of mesenchymal stem/stromal cell-derived small extracellular vesicles (MSC sEVs), comprising a viral vector encoding a calcitonin gene-related antagonist peptide (aCGRP).

82. The population of MSC sEVs of claim 81, wherein individual MSC sEVs are less than about 200 nanometers in diameter.

83. The population of MSC sEVs of claim 81, wherein the aCGRP comprises a truncated CGRP comprising SEQ ID NO: 1 or a sequence at least 90% identical to SEQ ID NO: 1.

84. The population of MSC sEVs of claim 81, wherein the aCGRP is operatively conjugated to a signal sequence.

85. The population of MSC sEVs of claim 84, wherein the signal sequence comprises a ssPAM signal sequence.

86. The population of MSC sEVs of claim 85, wherein the ssPAM signal sequence comprises SEQ ID NO: 3 or a sequence at least 90% identical to SEQ ID NO: 3.

87. The population of MSC sEVs of claim 81, wherein the viral vector is selected from a lentiviral vector, an adenoviral vector, or an adeno-associated vector (AAV).

88. The population of MSC sEVs of claim 87, wherein the AAV vector comprises AAV2.

89. A fusion polypeptide, comprising:
a) an ssPAM signal sequence comprising SEQ ID NO:3 or a sequence at least 90% identical to SEQ ID NO: 3, and
b) a CGRP₈₋₃₇ peptide comprising SEQ ID NO:1 or a sequence at least 90% identical to SEQ ID NO: 1.

90. A method of treating pain in a subject in need thereof, comprising administering to the subject a therapeutically effective amount of a pharmaceutical composition comprising the population of MSC sEVs of claim 81.

91. The method of claim 90, wherein the pain is selected from chronic pain, inflammatory pain, osteoarthritis pain, inflammation, spinal cord injury pain, headache, or neuropathic pain.

92. The method of claim 90, wherein the pharmaceutical composition is administered to the subject via a targeted injection route.

93. The method of claim 90, wherein the pharmaceutical composition is administered to the subject via a targeted intrathecal, intra-ganglionic, intra-spinal, intravitreal, intracochlear, intra-cerebral, or intra-articular injection.

94. The method of claim 90, wherein the pharmaceutical composition is administered to the subject in a single injection.

95. The method of claim 90, wherein the pharmaceutical composition is administered to the subject in more than one injection.

96. The method of claim 95, wherein subsequent administrations of the pharmaceutical composition are administered once every other day (Q2D), once every three days (Q3D), once every 4 days (Q4D), once per week (Q1W), once every 2 weeks (Q2W), once every three weeks (Q3W), once every 4 weeks (Q4W), once monthly, twice per month, once every other month, once every 6-months, once per year, or a combination thereof.

97. A method for reducing, preventing, or reversing inflammation in a subject in need thereof, comprising administering to the subject a therapeutically effective amount of a pharmaceutical composition comprising the population of MSC sEVs of claim 81.

98. A method of obtaining a population of mesenchymal stem/stromal cell-derived small extracellular vesicles (MSC sEVs) comprising a viral vector encoding a calcitonin gene-related antagonist peptide (aCGRP), comprising:
(i) obtaining a tissue sample of an infrapatellar fat pad (IFP) of a subject;
(ii) mechanically dissecting the IFP tissue sample;
(iii) washing the dissected IFP tissue sample in the presence of one or more digestion enzymes in a buffered solution;
(iv) inactivating the one or more digestion enzymes;
(v) culturing the digested IFP tissue sample in media to produce a population of IFP-MSCs;
(vi) transducing the population of IFP-MSCs with one or more viral vectors encoding a CGRP₈₋₃₇; and
(vii) performing one or more purification steps to isolate the population of MSC sEVs, wherein the one or more purification steps are selected from filtering, centrifuging, or a combination thereof.

99. The method of claim 98, wherein the aCGRP comprises a truncated CGRP peptide comprising SEQ ID NO: 1 or a sequence at least 90% identical to SEQ ID NO: 1.

100. The method of claim 98, wherein the viral vector comprises an AAV vector.
